(19) United States
(12) Patent Application Publication
(10) Pub. No.: US 2009/0210253 A1
(43) Pub. Date: Aug. 20, 2009

(54) METHOD AND SYSTEM FOR IDENTIFICATION AND MANAGEMENT OF PATIENTS FOR SLEEP DISORDERS

(76) Inventors: Carol E. ASH, North Brunswick, NJ (US); Jeffrey A. SANK, Hillsborough, NJ (US)

Correspondence Address: DIANE DUAN MCKAY, ESQ.;MATHEWS, SHEPHERD, MCKAY & BRUNEAU, P.A., SUITE 201, 29 THANET ROAD, PRINCETON, NJ, 08540, US

(21) Appl. No.: 12/266,653

(22) Filed: Nov. 7, 2008

Related U.S. Application Data
(60) Provisional application No. 61/002,184, filed on Nov. 7, 2007.

(51) Int. Cl. G06Q 50/00; A61B 5/11; A61B 5/08

(52) U.S. Cl. 705/3; 600/595; 600/529; 705/2

(57) ABSTRACT

The present invention provides a unique clinical workflow, scoring system, and system architecture for sleep specific applications. The present invention provides a unique logic system, identifying factors of sleep disorders and a sleepiness scale. The present invention comprises a logic system that incorporates the patient's i) medical history, ii) clinical evaluation, and iii) diagnostic testing data for directing patient care and clinical decision making. The system of the present invention incorporates new scoring systems to drive diagnostic testing and decision making. The present invention can also suggest and drive treatment options based on evidence based medicine, society guidelines, standards, consensus statements and clinical expertise.

### CROSS REFERENCE TO RELATED APPLICATION

This application claims the benefit of U.S. Provisional Patent Application No. 61/002,184, filed Nov. 7, 2007, the entirety of which is hereby incorporated by reference into this application.

### BACKGROUND OF THE INVENTION

1. Field of the Invention

The present invention relates to a method and system for the identification and management of patients for sleep disorders in which information is collected, scored, merged and interpreted to provide suggested diagnosis and treatments.

2. Description of the Related Art

Tracking mechanisms aimed at maximizing efficiency in tracking the needs of patient equipment and/or follow-up appointments have been described. Healthcare Information Systems contain integrated software applications for hospital-wide use. Examples of Healthcare Information Systems include products from the following manufacturers Cerner, Meditech, Epic, Eclipsys, McKesson and GE Centricity. Other market products provide tracking mechanisms aimed at maximizing efficiency in tracking the needs of patients equipment and/or follow-up appointments.

Braebon software provides a checklist, a database and electronic forms. American Academy of Sleep Medicine Compumedics Profusion software provides patient tracking of sleep disorders. The above-described applications do not provide logic and scoring mechanisms.

It is desirable to provide a system for sleep-specific applications including logic and scoring systems.

### SUMMARY OF THE INVENTION

The present invention provides a unique clinical workflow, scoring system, and system architecture for sleep specific applications. The present invention provides a unique logic system, identifying factors of sleep disorders and a sleepiness scale. The present invention comprises a logic system that incorporates the patient's i) medical history, ii) clinical evaluation, and iii) diagnostic testing data for directing patient care and clinical decision making. The system of the present invention incorporates new scoring systems to drive diagnostic testing and decision making. The present invention can also suggest and drive treatment options based on evidence based medicine, society guidelines, standards, consensus statements and clinical expertise.

The present invention has the advantage of allowing for efficient screening and management of large populations. The present invention can help minimize costs whilst maintaining outcomes and quality. The present invention provides for more efficient utilization of services and targeting of subpopulations for more effective treatment.

The invention will be more fully described by reference to the following drawings.

### BRIEF DESCRIPTION OF THE DRAWINGS

FIG. 1 is a schematic diagram of overview workflow 1 (WF1) for determining a system for screening patients for sleep disorder including interfaces and a logical progression through workflows of the present invention.

FIG. 2 is a schematic diagram of a screening module from workflow 2 (WF2).

FIG. 3A is a schematic diagram of a pre-encounter module from workflow 3 (WF3).

FIG. 3B is a schematic diagram of patient history acquisition template.

FIG. 4 is a schematic diagram of an encounter module from workflow 4 (WF4).

FIG. 5 illustrates a schematic diagram of a movement clinical encounter pathway from workflow 5 (WF5).

FIG. 6 illustrates a schematic diagram of insomnia clinical evaluation pathway from workflow 6 (WF6).

FIG. 7 illustrates a schematic diagram of breathing clinical evaluation pathway from workflow 7 (WF7).

FIG. 8 illustrates a schematic diagram of hypersomnia clinical evaluation pathway from workflow 8 (WF8).

FIG. 9 illustrates parasomnia clinical evaluation pathway from workflow 9 (WF9).

FIG. 10 illustrates circadian clinical evaluation pathway from workflow 10 (WF10).

FIG. 11 illustrates variant clinical evaluation pathway from workflow 11 (WF11).

FIG. 12 illustrates sleep disorders related to other conditions clinical evaluation pathway from workflow 12 WF12).

FIG. 13 illustrates diagnostic testing pathway (DTP) from workflow 13 (WF13).

FIG. 14 illustrates a schematic graph of diagnosis-to-treatment mapping module 1400 shown in workflow 14 (WF14).

FIG. 15 is a schematic diagram of PAP administration module described in workflow 15 (WF15).

FIG. 16 illustrates a schematic diagram of an encounter classification module from workflow 16 (WF16).

FIG. 17 illustrates a schematic diagram of emergency and medical (E&M) coding module from workflow 17 (WF17).

FIG. 18 is a schematic diagram of tracking module from workflow 18 (WF18).

FIG. 19 is a schematic diagram of research module from workflow 19 (WF19).

### DETAILED DESCRIPTION

Reference will now be made in greater detail to a preferred embodiment of the invention, an example of which is illustrated in the accompanying drawings. Wherever possible, the same reference numerals will be used throughout the drawings and the description to refer to the same or like parts.

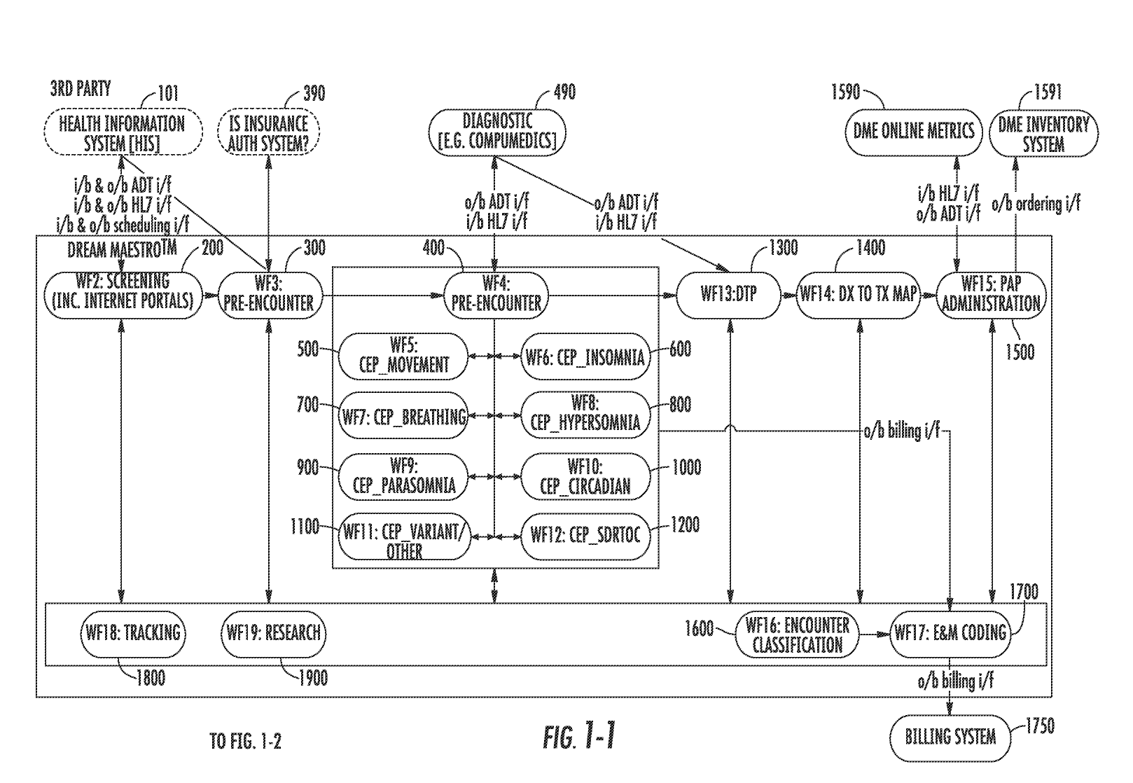

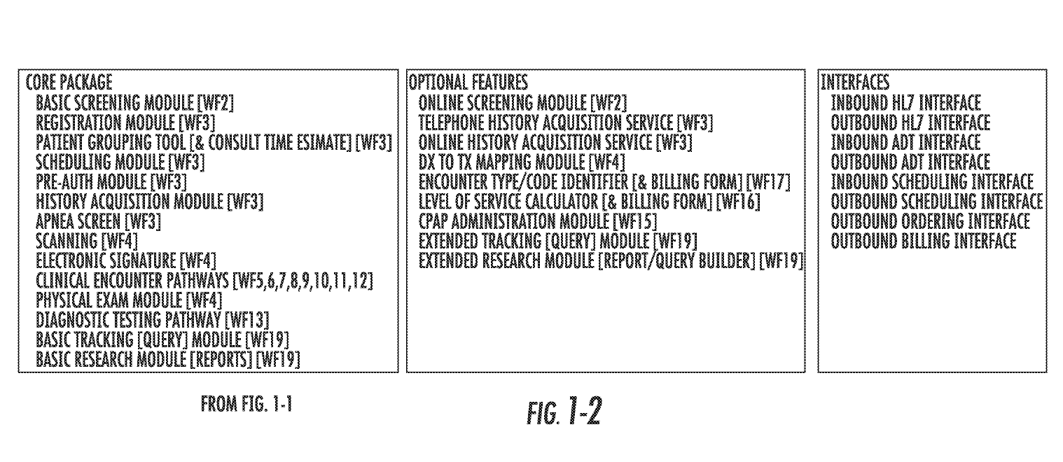

FIG. 1 is a schematic diagram of overview workflow 1 (WF1) for determining system 100 for screening patients for sleep disorders including interface and a logical progression through workflows of the present invention. Health information system (HIS) 101 interacts with screening module 200 from workflow 2 (WF2) and pre-encounter module 300 from workflow 3 (WF3). Pre-encounter module 300 interacts with encounter module 400 from workflow 4 (WF4). Data from pre-encounter module 300 can be used by insurance authorization system 390. Encounter module 400 diagnostic testing module 1300 can receive data from diagnostic system 490. Encounter module 400 uses one or more clinical encounter pathways (CEP) such as movement clinical encounter pathway module 500 from workflow 5 (WF5), insomnia clinical encounter pathway module 600 from workflow 6 (WF6), breathing clinical encounter pathway module 700 from workflow 7 (WF7), hypersomnia clinical encounter pathway module 800 from workflow 8 (WF8), parasomnia clinical encounter pathway module 900 from workflow 9 (WF9), circadian clinical encounter pathway module 1000 from workflow 10 (WF10), variant clinical encounter pathway module 1100 from workflow 11 (WF11) and sleep disorder related to other conditions (SDRTOC) clinical encounter pathway module 1200 from workflow 12 (WF12). Data from encounter module 400 is received in diagnostic testing pathway (DTP) from workflow 13 (WF13) for standardizing and management of the received data. Data from diagnostic testing pathway module 1300 is received in diagnosis to treatment mapping module 1400 from workflow 14 (WF14) for providing diagnosis and treatment of sleep conditions. Patient airway pressure administration module 1500 from workflow 15 (WF15) encounters classification module 1600 from workflow 16 (WF16) and E&M coding module 1700 from workflow 17 (WF17). Positive airway pressure administration module 1600 provides data to delivery of medical equipment (DME) online metrics 1590 and DME inventory system 1591. B&M coding module 1700 provides data to billing system 1750. Tracking module 1800 from workflow 18 (WF18) provides electronic patient tracking through all workflows. Research module 1900 from workflow 19 (WF19) provides a central repository for patient electronic data for potential research initiatives and patient profiles.

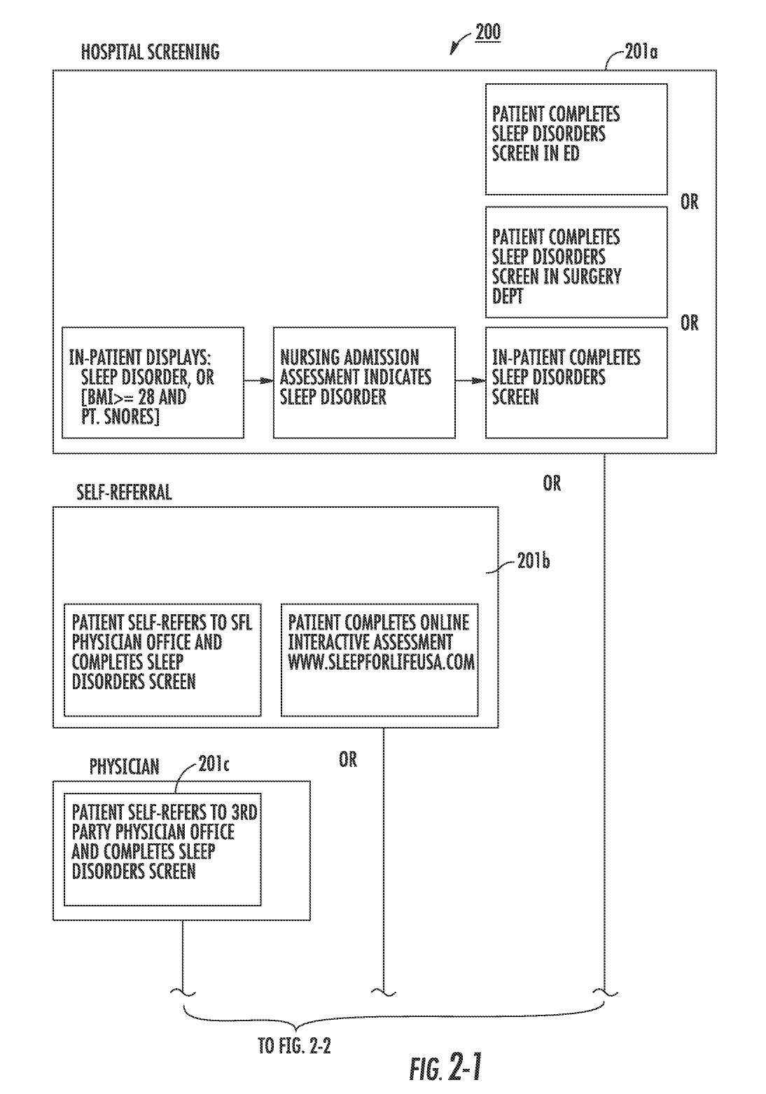

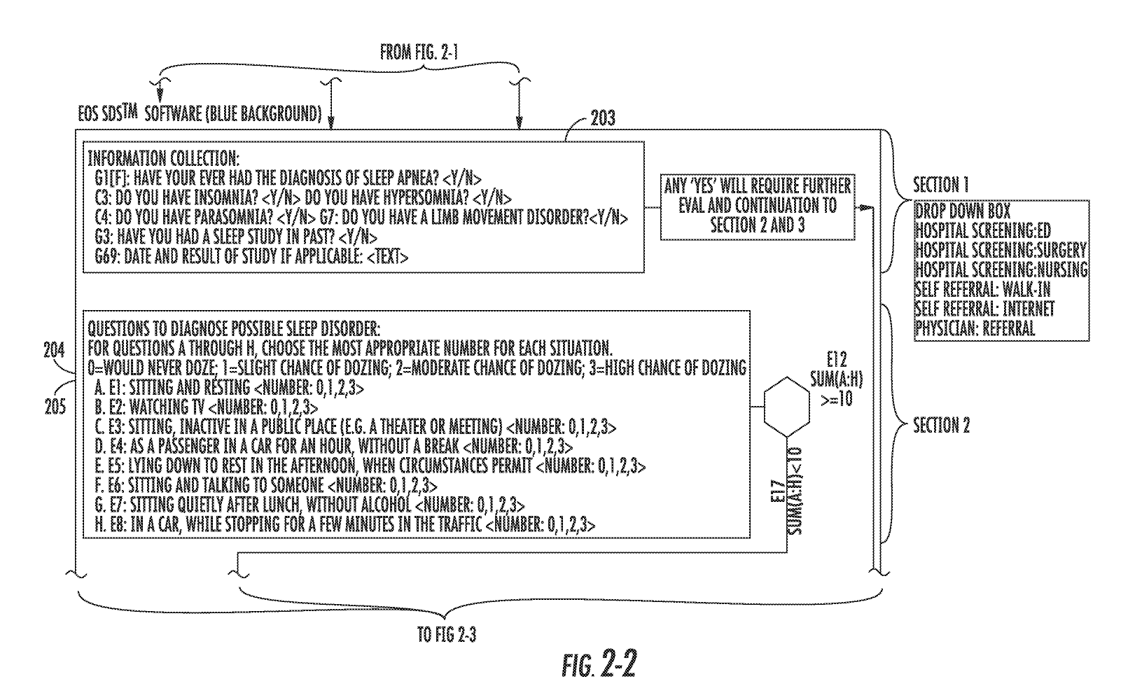

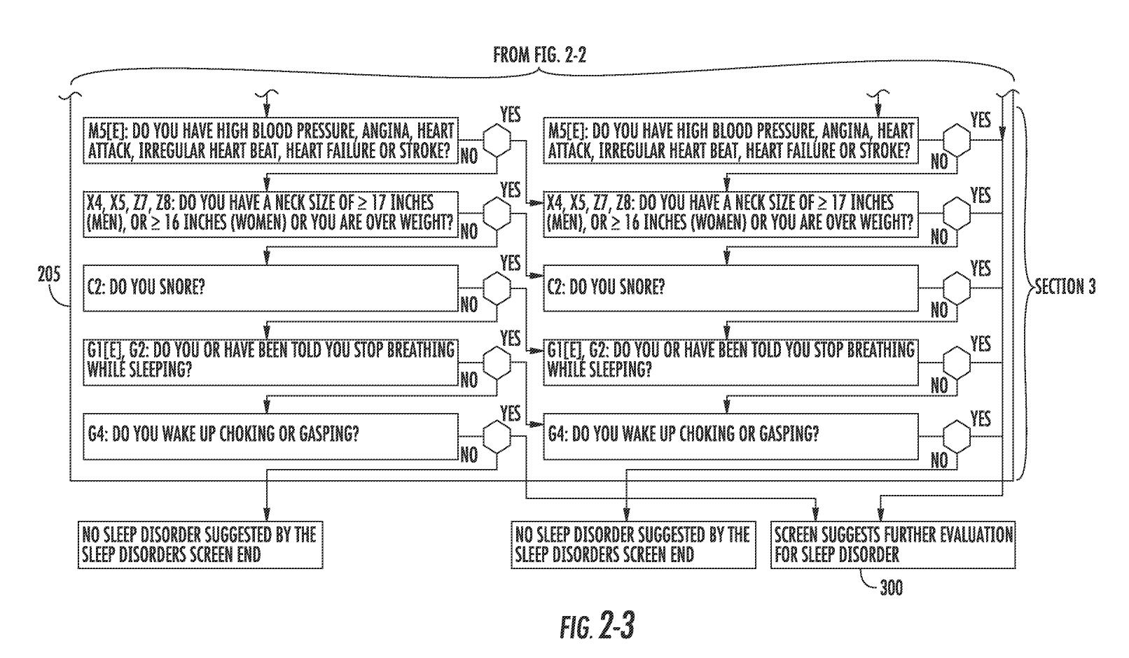

FIG. 2 is a schematic diagram of workflow 2 (WF2). Screening module 200 can be accessed electronically via the Internet or through an interface to sleep disorder screening module 200. Alternatively, screening forms can be supplied by a patient or physician and manually entered, scanned in a file or accessed over the Internet for input into sleep disorder screening module 200. A plurality of multiple practitioners and/or patients engage hospital screening module 201a, self reference module 201b or physician module 201c in order to screen for potential risk factors for sleep disorders.

Screening module 200 interacts with the information collection module 203 to collect information of a patient directed to sleep complaint disorders such as insomnia, parasomnia, hypersomnia, limb movement disorder, and/or having a positive history of sleep apnea. A positive response to a complaint of the patient of any of the aforementioned disorders in information collection module 203 generates the need for further sleep evaluation and continuation to sleepiness scale module 204. Sleepiness scale module 204 provides an electronic format in a computer scoring system which generates a numeric score for excessive daytime sleepiness. Sleepiness scale module 204 can include a template of a predetermined number of questions to diagnose a sleep sleepiness and numeric answers. Example questions of sleepiness and numeric answers include sitting and reading <number: 0, 1, 2, 3>; watching television <number: 0, 1, 2, 3>; sitting, inactive in a public place (e.g., a theatre or meeting)<number: 0, 1, 2, 3>; as a passenger in a car for an hour, without a break <number: 0, 1, 2, 3>; lying down to rest in the afternoon, when circumstances permit <number: 0, 1, 2, 3>; sitting and talking to someone <number: 0, 1, 2, 3>; sitting quietly after lunch, without alcohol <number: 0, 1, 2, 3>; in a car, while stopping for a few minutes in the traffic <number: 0, 1, 2, 3>. An appropriate numeric answer for each situation is selected by the user. For example, the following scale can be used would: 0=never doze; I=slight chance of dozing; 2=moderate chance of dozing; 3=high chance of dozing. If the numeric score for daytime sleepiness is above a threshold, for example, if the score is less than 10, the patient continues to evaluation module 206. In evaluation module 206 a positive response to a predetermined number of questions to diagnose a possible sleep disorder, such as at least 2 of 5 questions, determines continuing to further evaluation in pre-encounter module 300. If the score is greater than or equal to 10, the patient continues to evaluation module 206 and a positive response to a smaller number of questions, such as only one question, determines continuing to further evaluation in pre-encounter module 300. Example questions to diagnose a possible sleep disorder include: Do you have high blood pressure, angina, hear attack, irregular heart beat, heart failure, or stroke?; Do you have a neck size of ≧17 inches (men), or ≧16 inches (women) or are you over weight?; Do you snore?; Do you or have you been told you stop breathing while sleeping?; Do you wake up choking or gasping?. All patients determined to meet the guidelines for further evaluation for a sleep disorder and are entered into pre-encounter module 300.

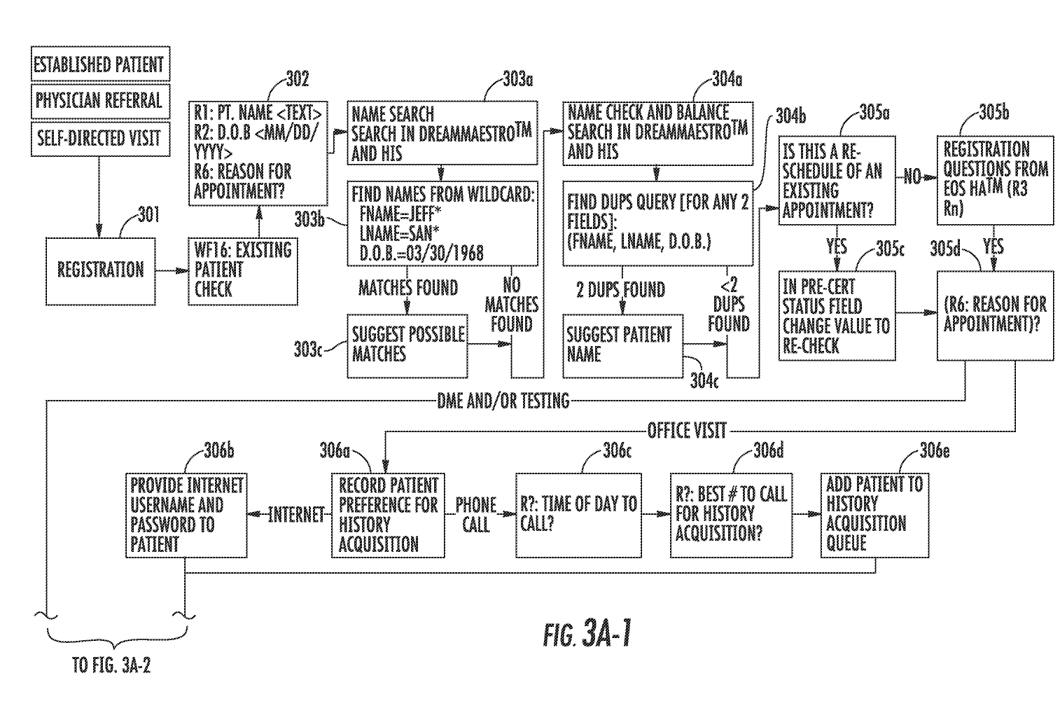

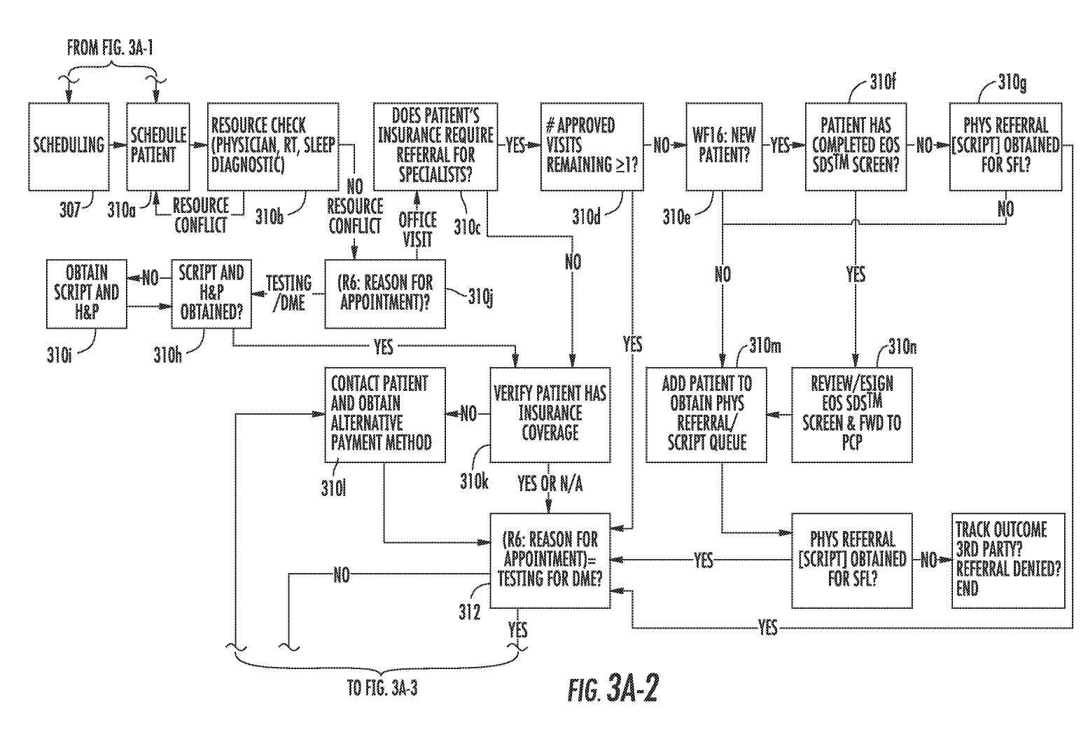

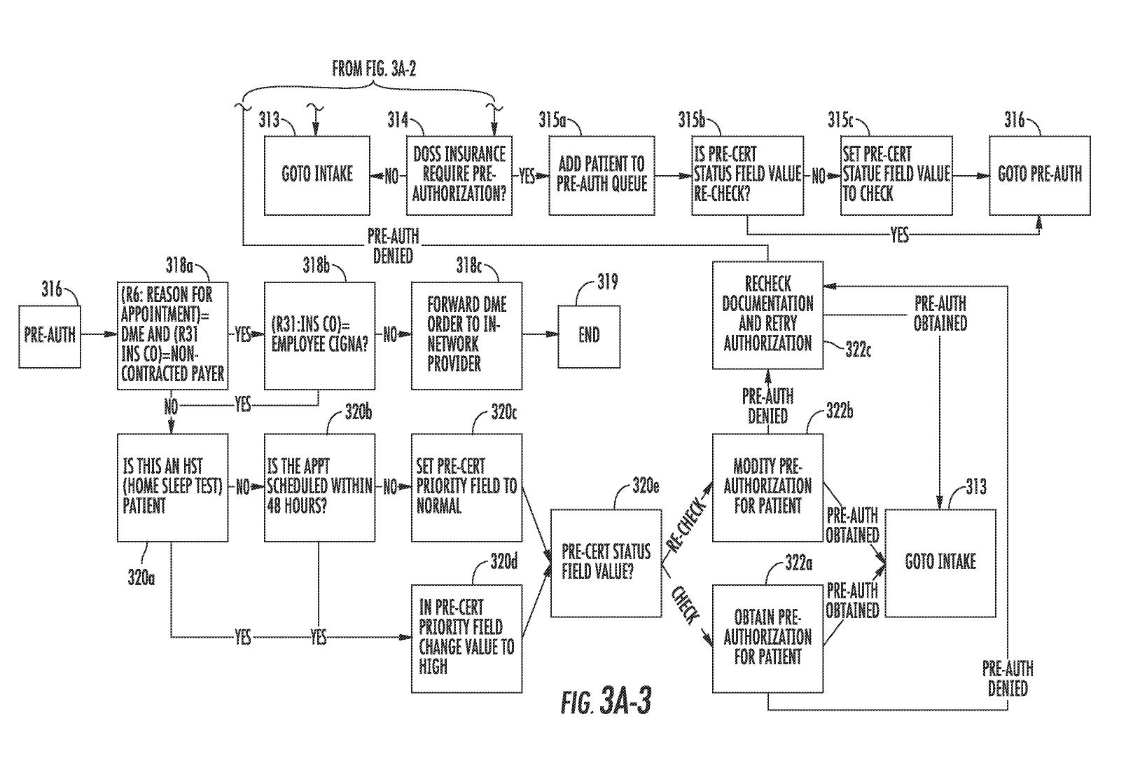

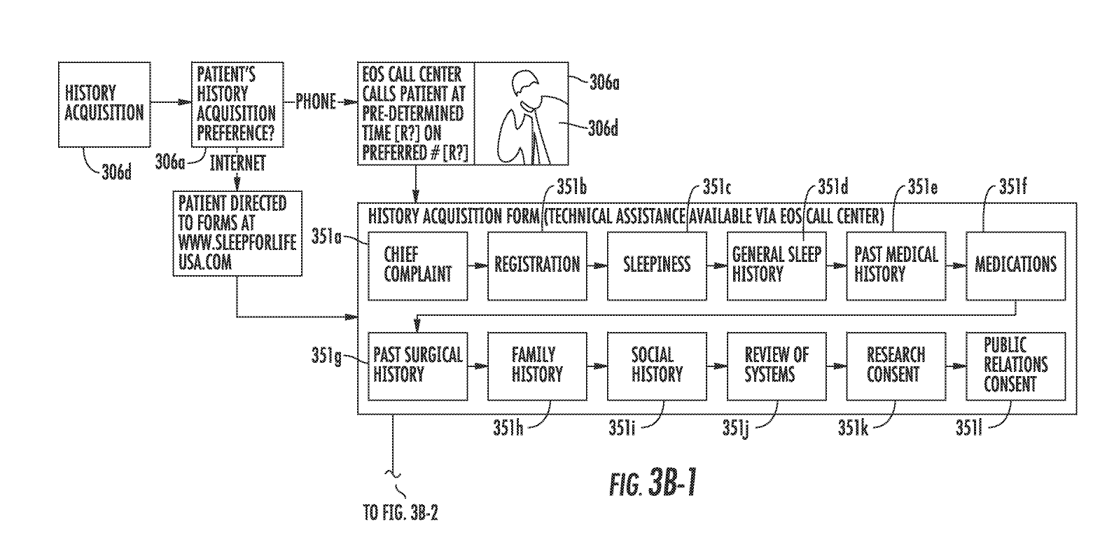

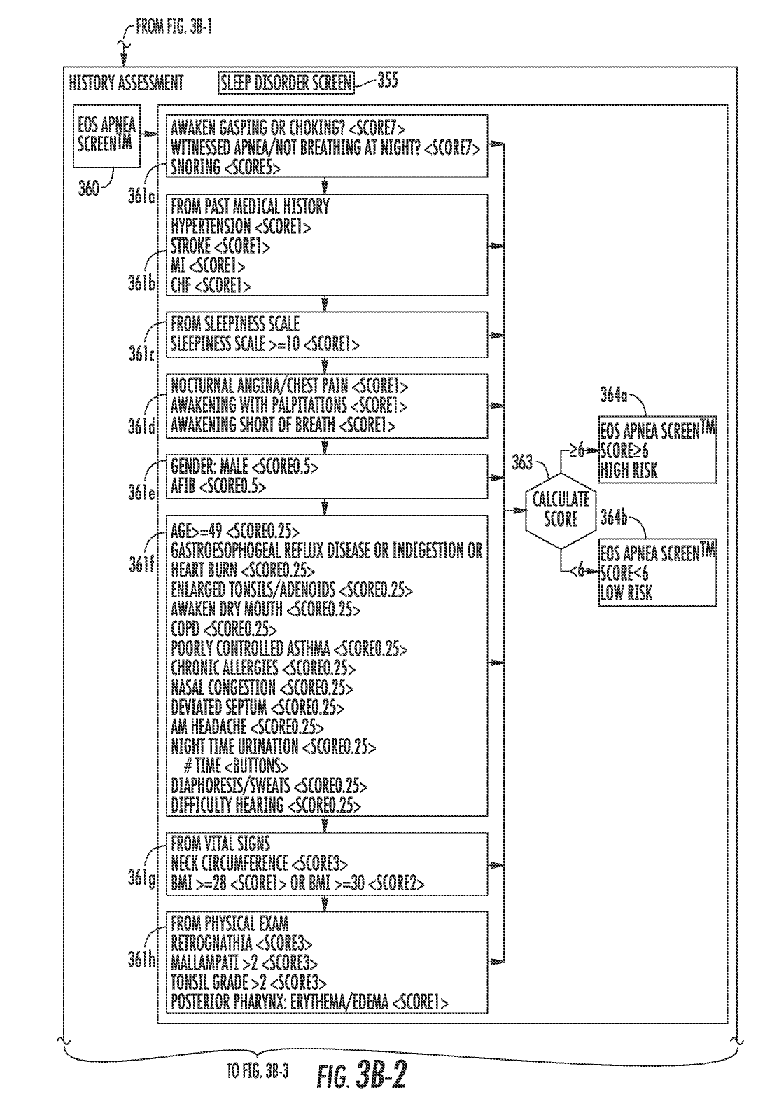

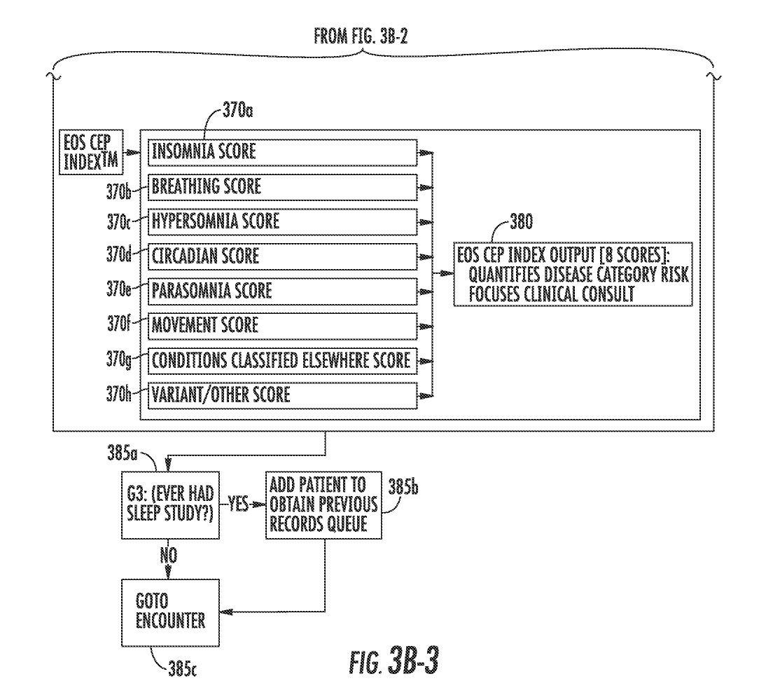

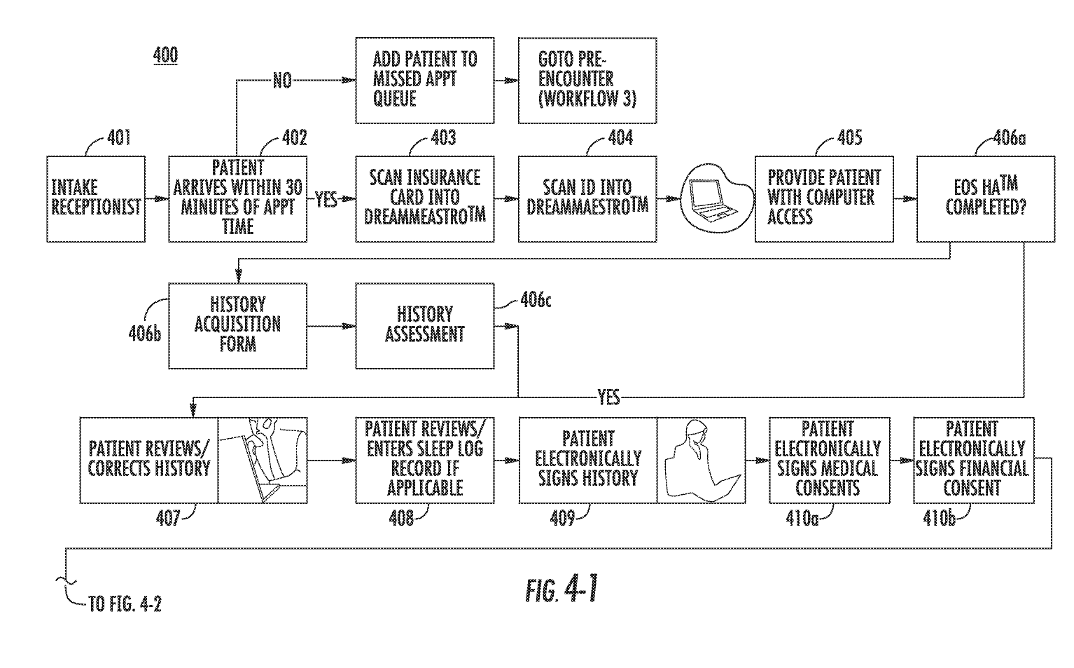

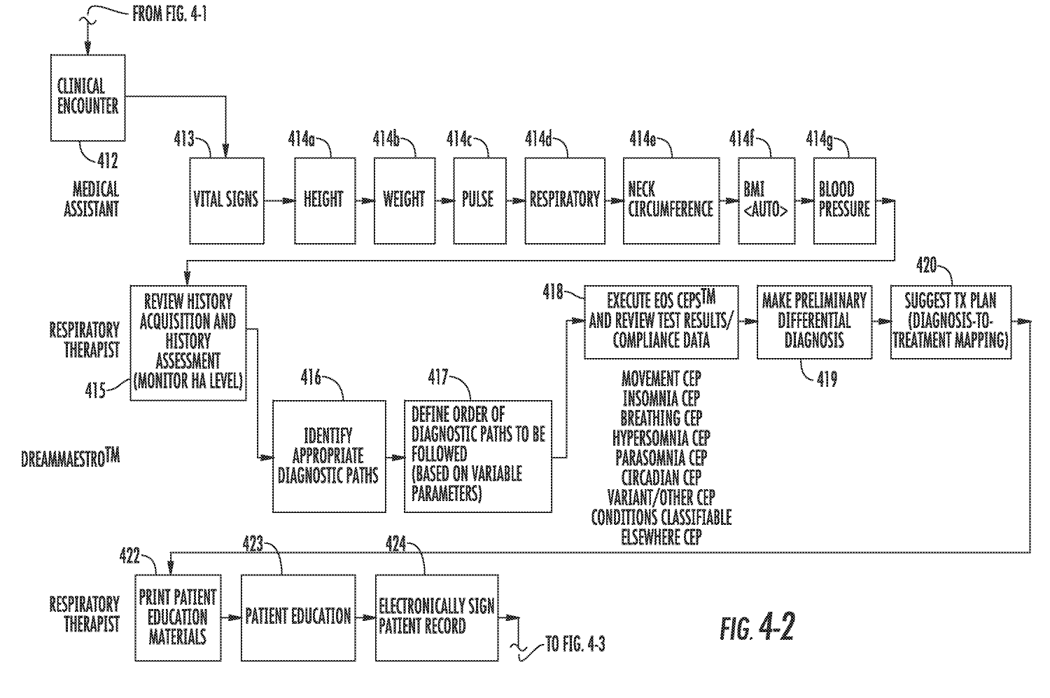

FIG. 3A is a schematic diagram of pre-encounter module 300 from workflow 3 (WF3). Pre-encounter module 300 completes pre-registration and forwards the registration data to healthcare information system (HIS) 101. This module identifies requirements defined by insurance payers to direct further patient initiation into a sleep clinical program or to an outside sleep specialist. Patient information including demographics and reason-for-visit are collected electronically from registration module 301. For example, information on the patient's name and the reason for the visit can be collected in block 302. Examples of a reason-for-visit include initial consultation to address chief complaint, test follow-up, treatment follow-up, equipment issues, and other issues. A name search for finding an existing name can be determined from blocks 303a-303c. The name can be checked in blocks 304a-304c for determining if there are duplicate entries and suggests the patient name. It is determined in blocks 305a-305d if the encounter is a schedule of a new appointment or an existing appointment and registration questions input is used to determine the reason for an appointment.

In blocks 306a-306d, the patient's preference for entering a history acquisition is determined. The patient's history can be acquired via either a sleep program staff phone call to electronically complete history acquisition template 350 or by direct access of the patient to patient history acquisition template 350 via the Internet. In block 306e, the patient history acquisition template 350 can be acquired. Patient history acquisition template can be acquired using a graphical user interface.

Electronic patient history acquisition template 350 includes patient's history information, as shown in FIG. 3B. Example information is shown in blocks 351a-351n which information comprises chief complaint, registration, sleepiness, sleep history, review of systems, past medical history, past surgical history, medications, allergies, research, consent, family history, social history, review of symptoms and public relations consent. Within electronic patient history acquisition template 350 is a sleep disorder screen (SDS) 355 to provide a general sleep history obtained utilizing specific questions designed to identify specific sleep disorders. Apnea Screen template 360 incorporates attributes or questions 361a-361n and numeric scoring system calculator 363 that identifies a patients risk in blocks 364a or 364b for sleep related breathing disorder, such as apnea.

History assessment can also include determining one or more clinical encounter pathways scores for various conditions. Example clinical encounter pathway scores include insomnia score 370a, breathing score 370b, hypersomnia score 370c, circadian score 370d, parasornmia score 370e, movement score 370f, conditions classified elsewhere score 370g and variant score 370h. In one embodiment, an encounter pathway index score 380 is determined from the eight scores 370a-370h. The outcome of questions from history acquisition 306a and/or electronic patient history acquisition template 350 and the apnea score from numeric scoring system 363 calculate and index score 380 can be used to initiate further evaluation of the patient in blocks 385a-385c and direct the patient to pre-encounter module 300.

Referring to FIG. 3A, if the reason for the appointment meets appointment guidelines for DME or testing, the patient is then scheduled for an appointment within HIS scheduling module 307. Block 310a-310n determine the need for insurance pre-certification and prompts pre-certification acquisition where required. In block 312, if the reason for appointment is not testing or DME, go-to-intake is performed in block 313. If the reason for the appointment is testing or DME, it is determined if the insurance requires pre-authorization in block 314. The patient is added to a pre-authorization queue and the status checked in block 315a-315c before going to go to pre-authorization block 316. Blocks 318a-318c are performed to provide a pre-authorization order in block 319. In blocks 320a-320d a pre-certification status field value is determined for certain candidates. In blocks 322a-322c, the pre-certification status is checked or rechecked until obtained in go-to-intake in block 313.

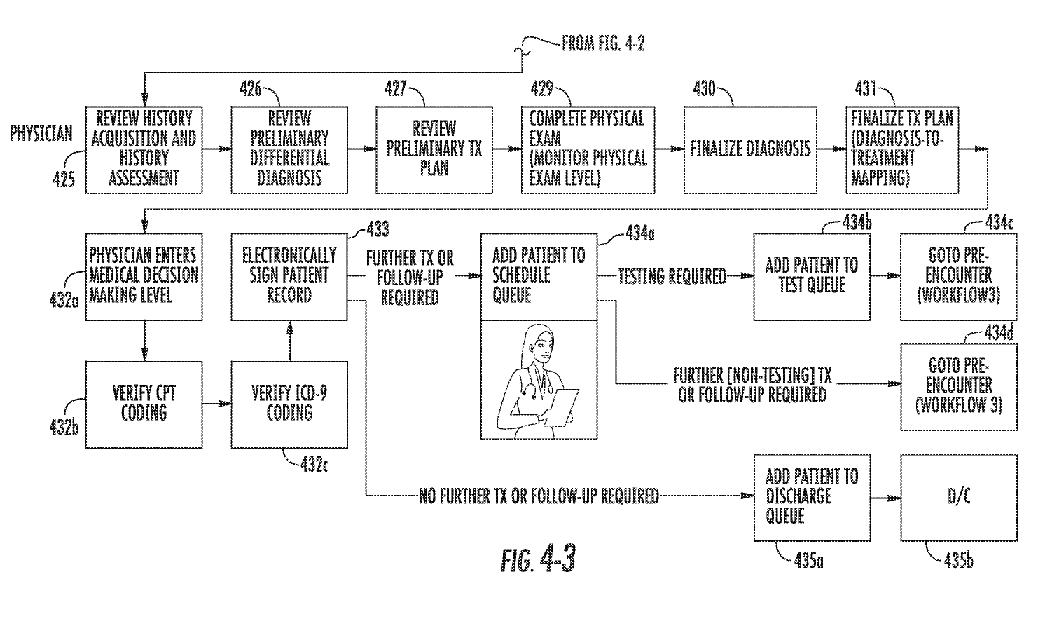

FIG. 4 is a schematic diagram of encounter module 400 from workflow 4 (WF4). In-take block 401 is accessed from go-to-intake block 313 or by a receptionist. A patient arrives within a predetermined time of an appointment in block 402. Data from an insurance card is entered in block 403, such as by electronically scanning the insurance card. An identification of the patient 403, such as established from block 306b, is entered in block 404. In block 405, a patient can access data using a computer.

All available history HIS data inclusive of the sleep disorder screen (SDS) 355 history acquisition (HA) template 350 and apnea screen (AS) template 360 can be accessed electronically from the central repository in blocks 406a-406c. The patient is then asked to review and correct all available data history data (HIS) in block 407. In block 408, the patient can enter a sleep log record if applicable. The patient electronically signs the available history HIS data for verification of accuracy in block 409. The patient can also be asked to electronically sign consents, such as for financial, treatment authorization, participation in research, and participation in public relations initiatives in blocks 410a-410b.

The patient continues to the clinical encounter module 412. Alternatively, clinical encounter module 412 is accessed from go-to-encounter module 385, shown in FIG. 3. Within clinical encounter module 412, the patient first encounters a medical assistant, who enters vital sign data 413 into the patient electronic record within HIS 101. Example vital signs data can include data described in blocks 414a-414g, such as height, weight, pulse, respiratory, body mass index (BMI), and blood pressure.

In block 415, the patient can encounter the respiratory therapist (RT). The RT can assess HIS 101 which includes all data entered via history acquisition template 350 including sleep disorder screen 355, apnea screen template 360 and vital signs from block 413a-413g and enter the data into an RT data collection form. The RT data collection form can assist the RT in identifying appropriate diagnostic pathways to be followed in block 416, identifying order of diagnostic in pathways (CEP) block 417 and selecting appropriate clinical encounter pathways in block 418.

Once the RT completes patient evaluation using the clinical encounter pathways, the RT can review the patients' sleep habits. Upon review of all available clinical data, and application of clinical expertise, the RT can provide a preliminary differential diagnosis in block 419 and a suggested treatment plan in block 420.

In block 422, patient education materials can be printed based upon the preliminary differential diagnosis and suggested treatment plan. In block 423, the patient education materials can be distributed and/or reviewed with the patient. In block 424, the RT electronically signs the patient record.

In block 425, the patient encounters a sleep physician (SDr). In blocks 425-427, the SDr can access HIS 101 which includes all data entered including: history acquisition template 350, sleep disorder screen 355, apnea screen 360, vital signs from block 413, CEP from block 418, the preliminary differential diagnosis and the suggested treatment plan from blocks 419 and 420 and enter the data into a physical exam data collection form. The SDr executes in block 429 a physical exam based on data collection form 428. At this point, the physician has all required information to complete a preliminary diagnosis in block 430. The SDr finishes the treatment plan and diagnosis in block 431. In block 432a-432c, the physician enters the medical decision and verifies the coding used in the patient record. In block 433, the physician electronically signs the physical exam data collection form. This will prompt a decision for either diagnostic testing, treatment in blocks 434a-434d or discontinuation of assessment in blocks 435a-435b.

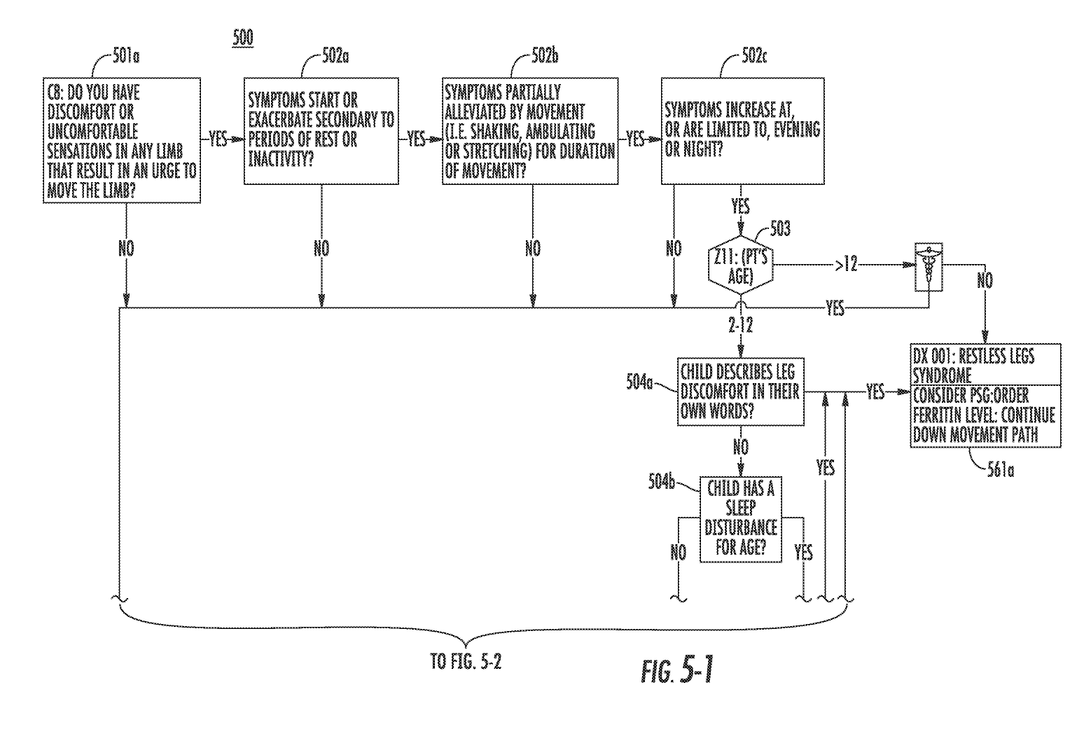

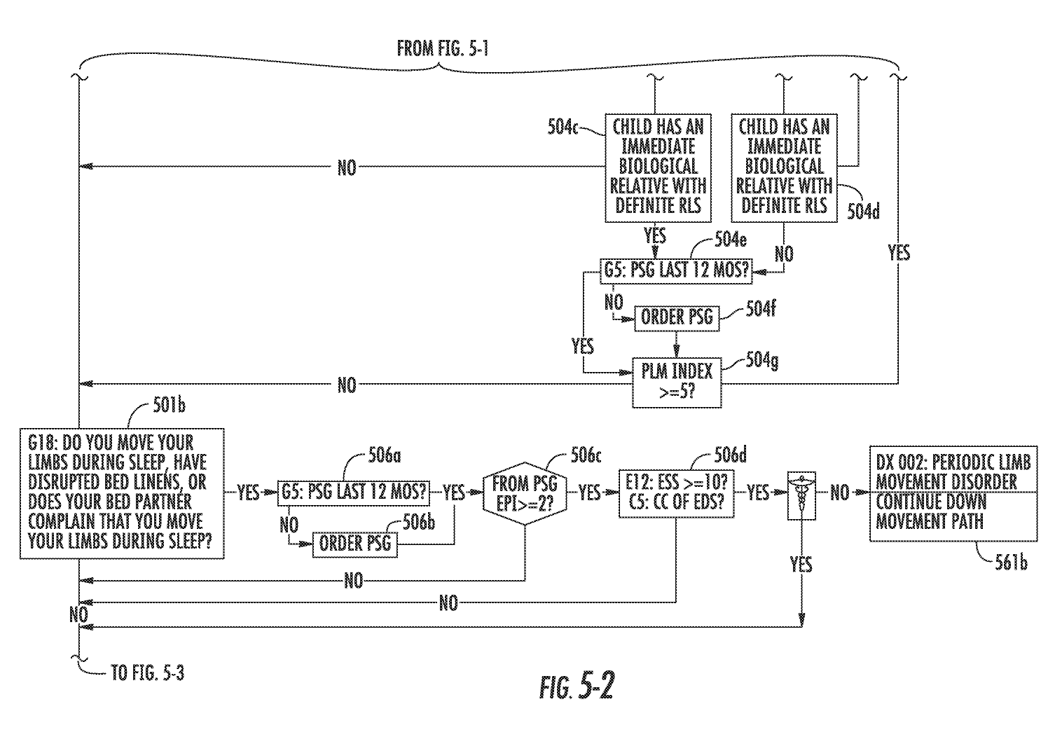

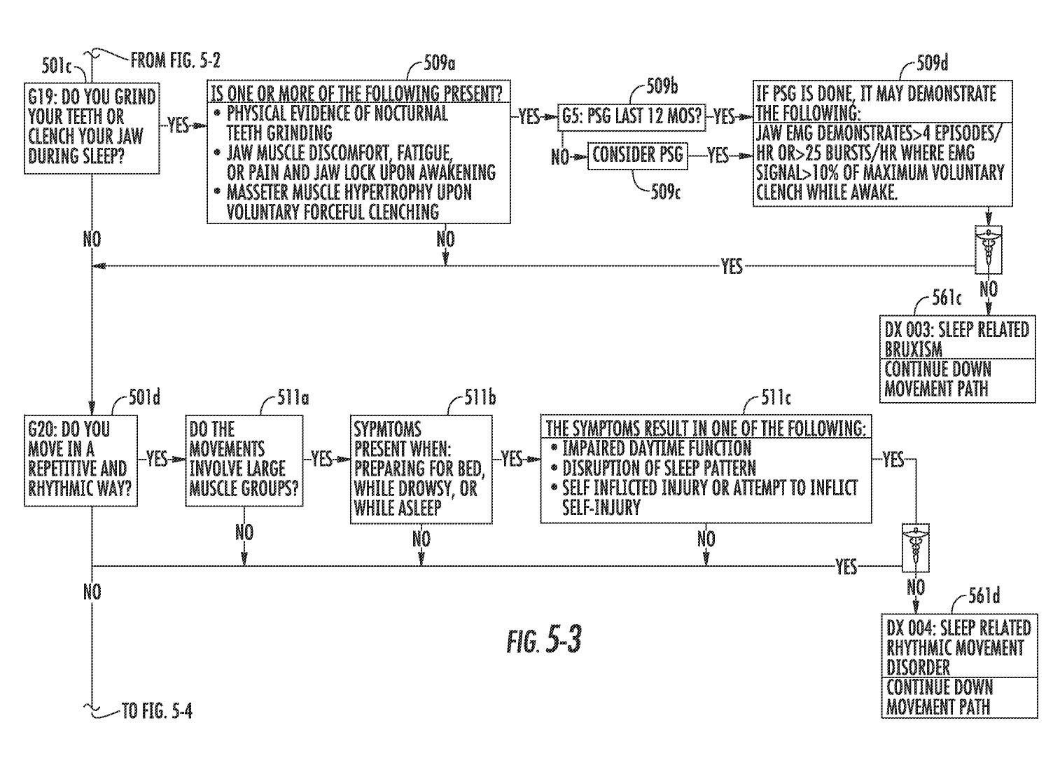

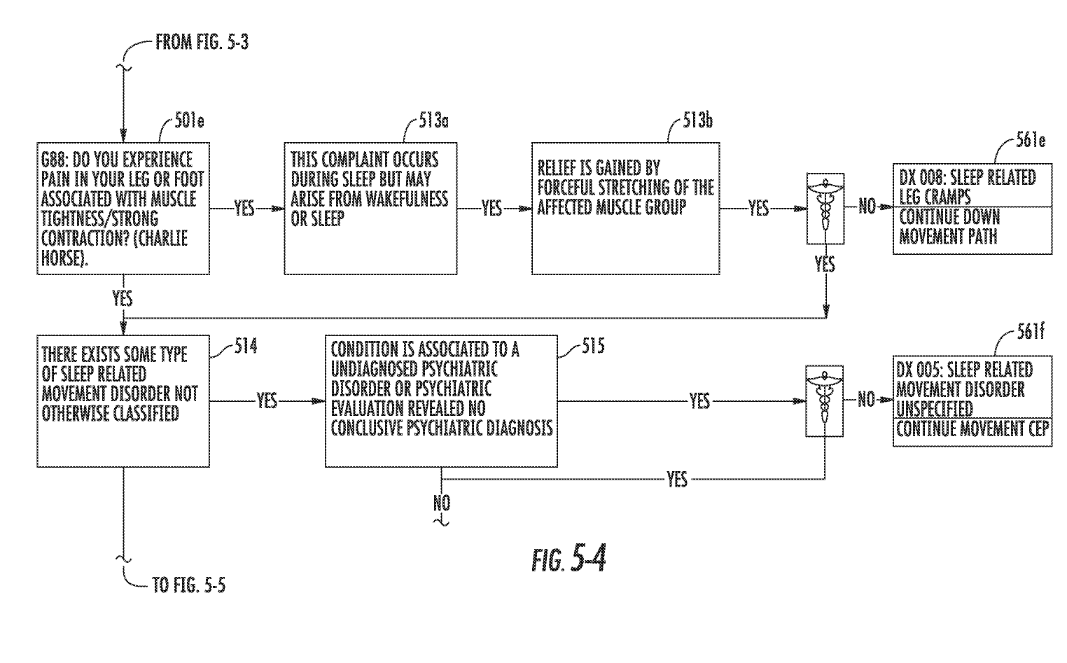

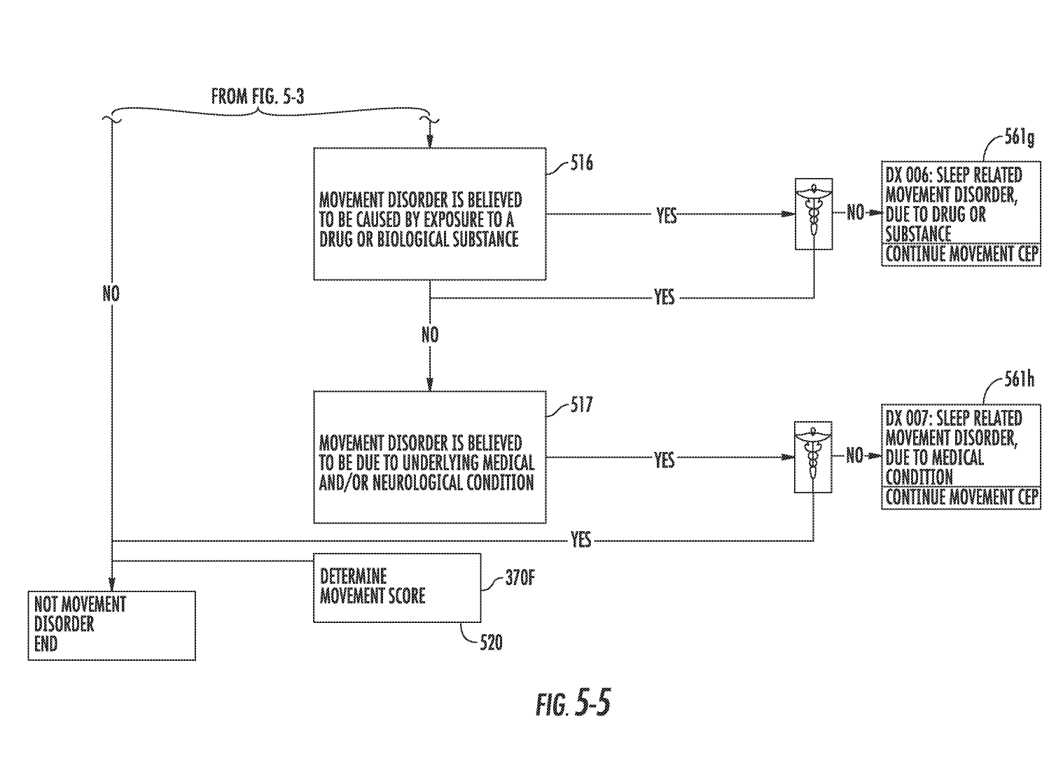

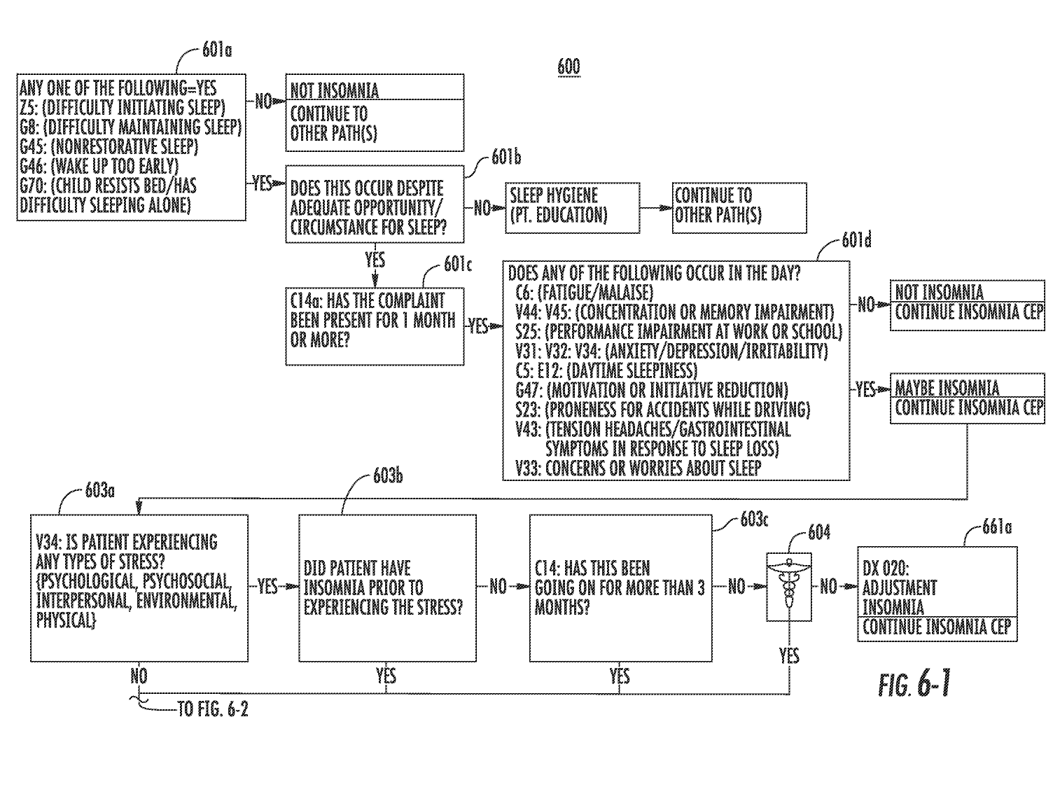

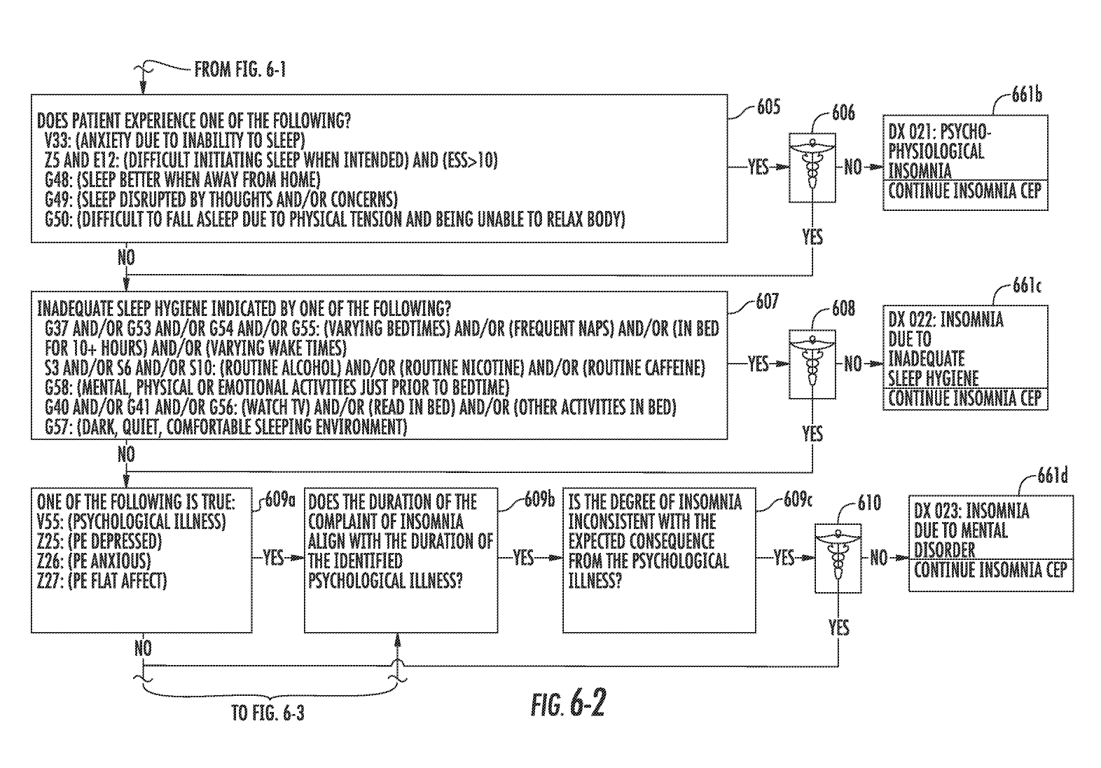

FIG. 5 illustrates a schematic diagram of movement clinical encounter pathway 500 from workflow 5 (WF5). One or more questions 501a-501r are used for further diagnostic data collection for determining respective diagnoses 561a-561h and determining a movement score 370f in block 520. For example, question 501a can relate to: Do you have discomfort or uncomfortable sensations in any limb that result in an urge to move the limb?. Blocks 502a-502c further determine the symptoms. Block 503 is a determination of the patient's age. If the patient is determined to be a child in block 503, blocks 504a-504g are determined for determining if a diagnosis of a child has restless leg syndrome in block 561a. If the patient is an adult symptoms 502a-502c are determined for determining a diagnosis of adult restless leg syndrome in block 561a. For example, questions 501b can relate to: Do you move your limbs during sleep, have disrupted bed linens, or does your bed partner complain that you move your limbs during sleep?. Polysysmograph (PSG) symptom data of blocks 506a-506d are determined for determining a diagnosis of a periodic limb movement disorder in block 561b. For example, question 501c can relate to: Do you grind your teeth or clench your jaw during sleep?. Symptom data and PSG data of blocks 509a-509d are determined for determining a diagnosis of a sleep related buxism in block 561d. For example, question 501d can relate to: Do you move in a repetitive and rhythmic way?. Blocks 511a-511b presenting questions of related symptoms are performed for determining a diagnosis of a sleep related rhythmic movement disorder in block 561d. For example, question 501e can relate to: Do you experience pain in your leg or foot associated with muscle tightness/strong contraction? (Charlie Horse). Blocks 513a and 513b are determined for muscle related symptoms for determining a diagnosis of a sleep related leg cramp disorder in block 516e. If there exists some type of unclassified disorder in block 514, block 515 is performed to determine a condition associated with an undiagnosed psychiatric disorder or psychiatric evaluation revealed no conclusive psychiatric diagnosis and determine a diagnosis of an unspecified sleep related movement disorder in block 561f. In block 516, a determination is made if the movement is caused by exposure to a drug or biological substance for determining a diagnosis of a sleep related movement disorder due to drug or substance in block 561g. In block 517, a determination is made if the disorder is due to an underlying medical or neurological condition for determining a diagnosis of a sleep related movement disorder due to a medical condition in block 561h.

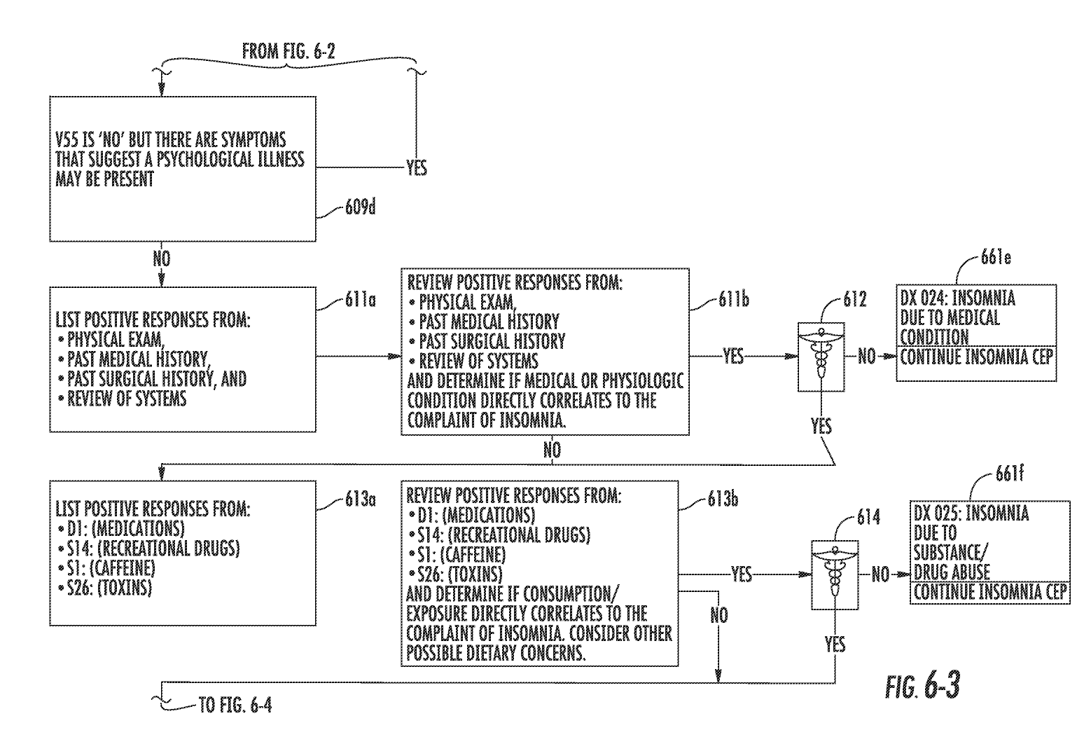

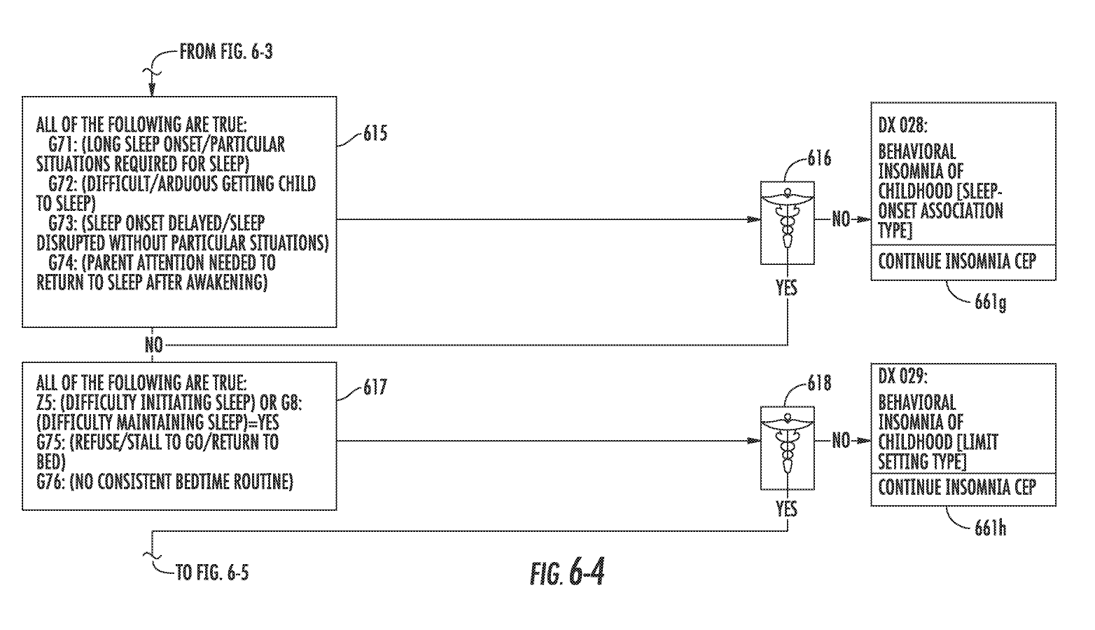

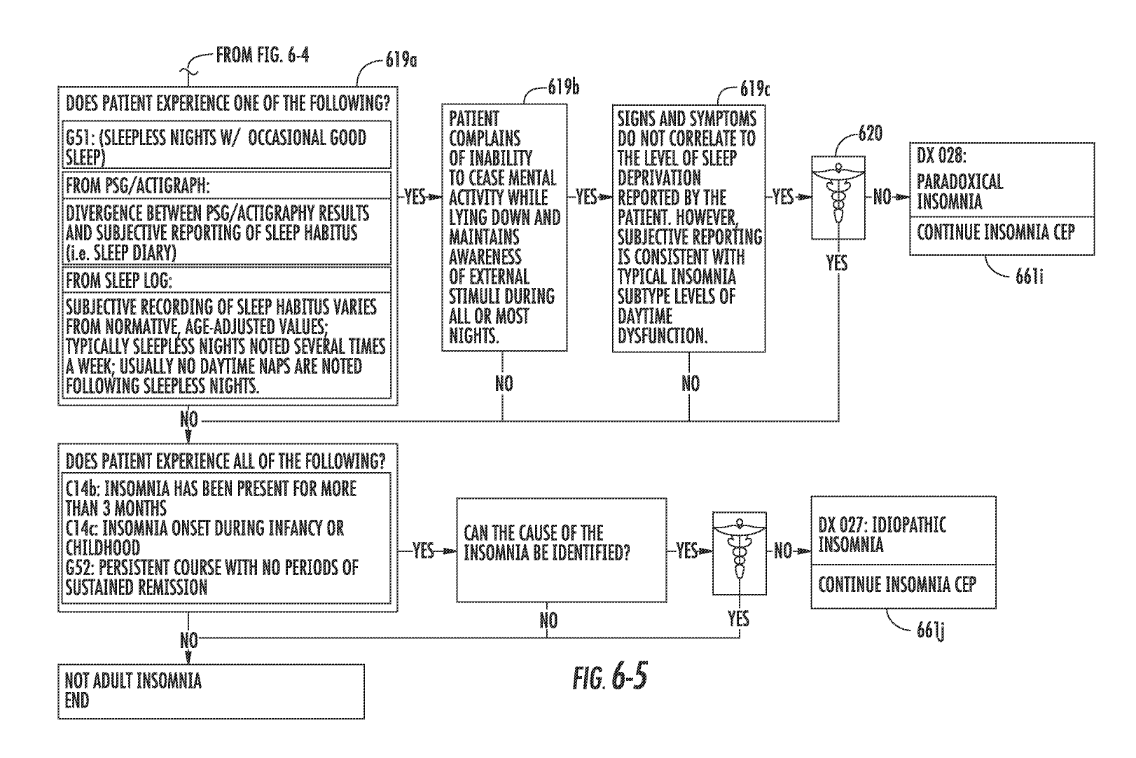

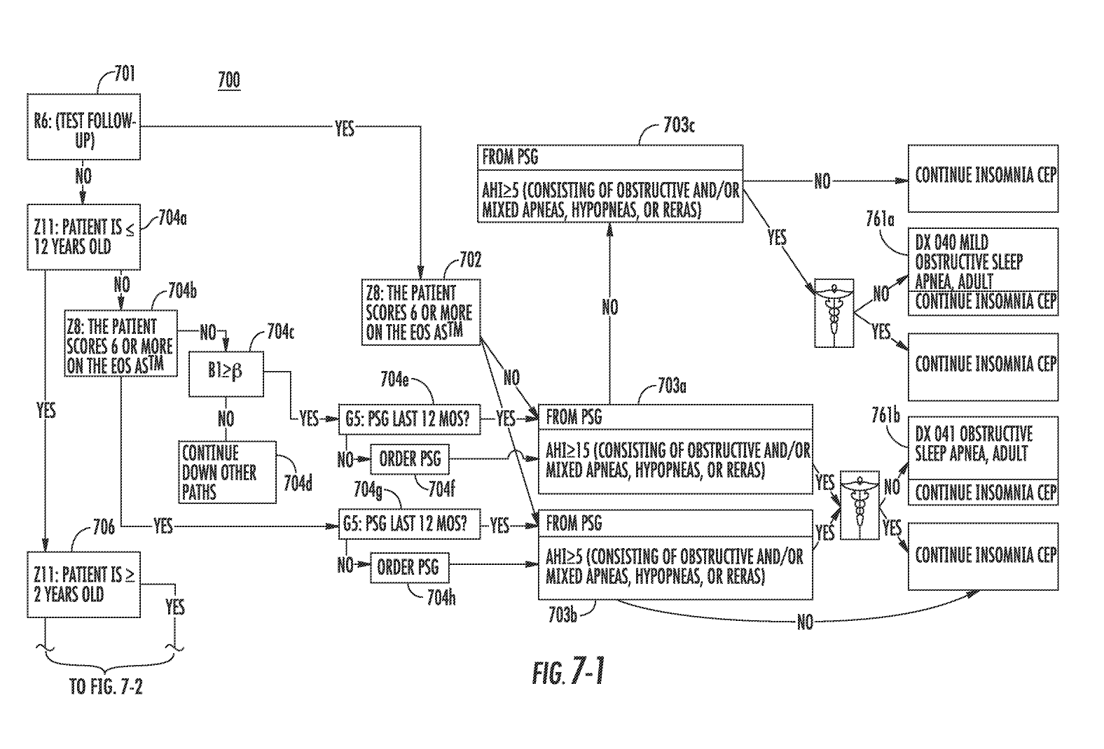

FIG. 6 illustrates a schematic diagram of insomnia clinical evaluation pathway 600 from workflow 6 (WF6). One or more preliminary questions or conditions 601a-601d are used for preliminary screening of insomnia. One or more questions 603a-603c provide further diagnostic data collection for determining a diagnosis for adjustment insomnia 661a from treatment decision block 604 or continuing to question 605. One or more questions 605 with multiple possible responses provides further diagnostic data collection for determining a diagnosis for psyco-physiological insomnia 661b from treatment decision block 606 or continuing to question 607. One or more questions 607 with multiple possible responses provides further diagnostic data collection for determining a diagnosis of insomnia due to inadequate sleep hygiene 661c from treatment decision block 608 or continuing to question 609a. One or more questions 609a-609d can be used with multiple possible responses provides further diagnostic data collection for determining a diagnosis of insomnia due to mental disorder 661d from treatment decision block 610 or continuing to medical response block 611a. Medical response block 611a and medical review of positive responses block 611b provide further diagnostic data collection for determining a diagnosis of insomnia due to a medical condition 661e from treatment decision block 612 or continuing to substance responses block 613a. Substance responses block 613a and substance review of responses block 613b provide further diagnostic data collection for determining a diagnosis of insomnia due to a diagnosis of substance/drug abuse 661f from treatment decision block 614 or continuing to sleep onset responses block 615. Sleep onset response block 615 provides further diagnostic data collection for determining a diagnosis of behavioral insomnia of childhood sleep onset 661g from treatment decision block 616 or continuing to setting responses block 617. Setting responses block 617 provides further diagnostic data collection for determining a diagnosis of behavioral insomnia of childhood type setting 661h for treatment decision block 618 or continuing to sleep symptoms block 619a. Sleep symptoms blocks 619a-619c provide further diagnostic data collection for determining a diagnosis of paradoxical insomnia 666i from treatment decision block 620 or continuing to patient experience block 621a and block 621b provide further diagnostic data collection for determining a diagnosis of idiopathic insomnia 661j.

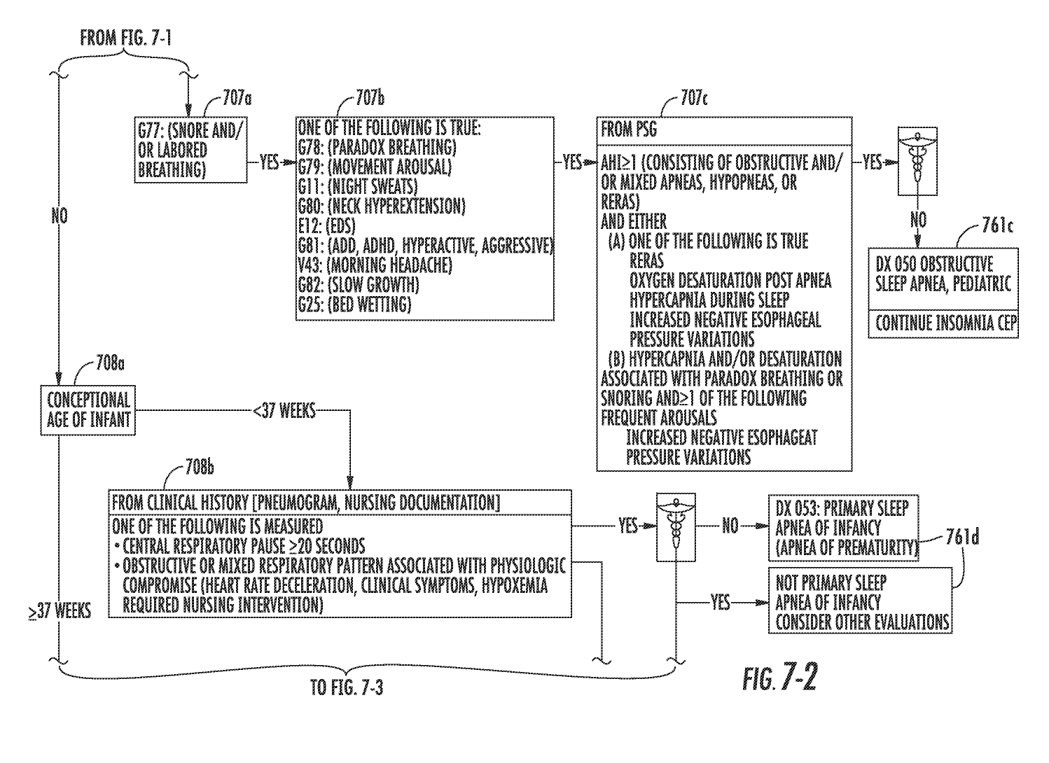

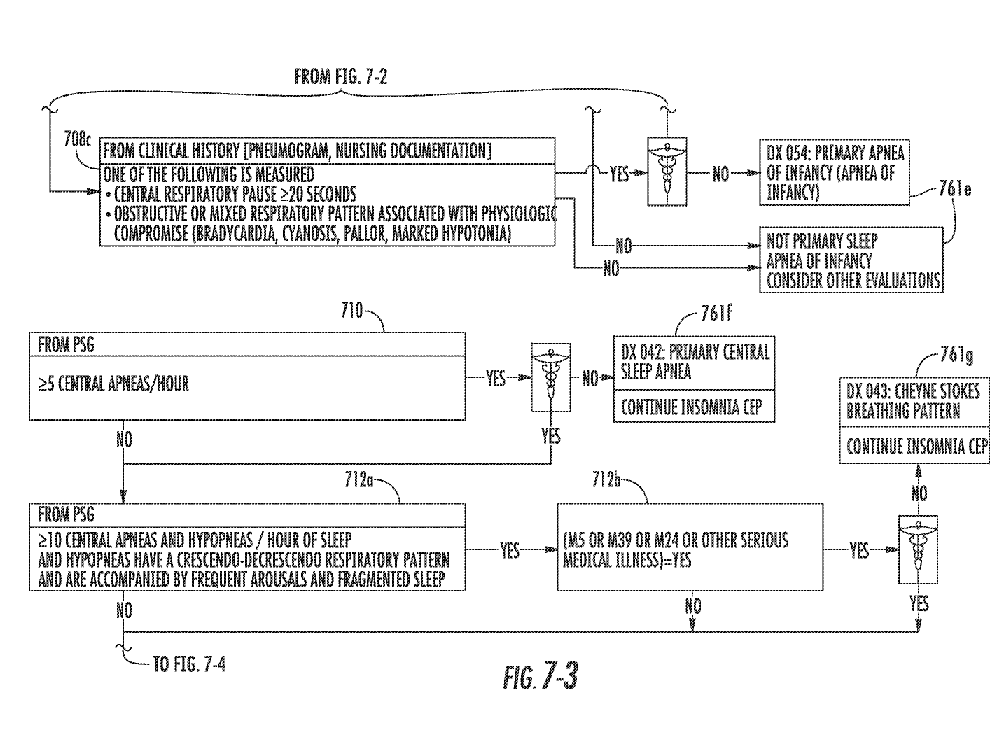

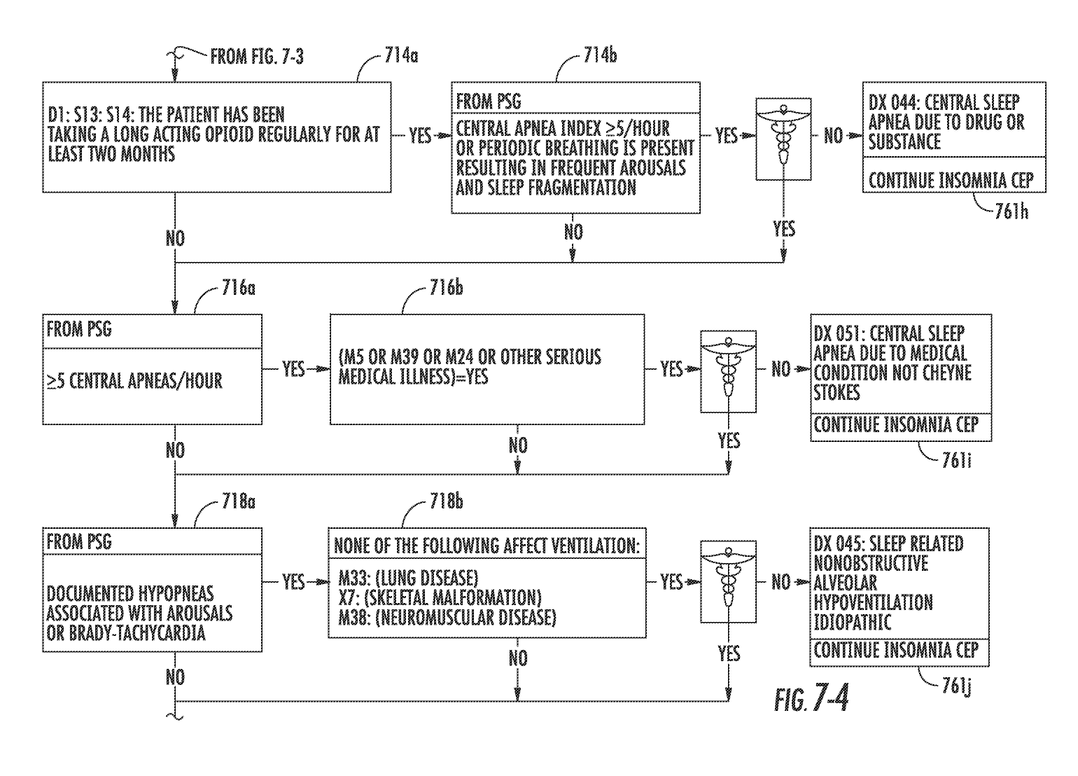

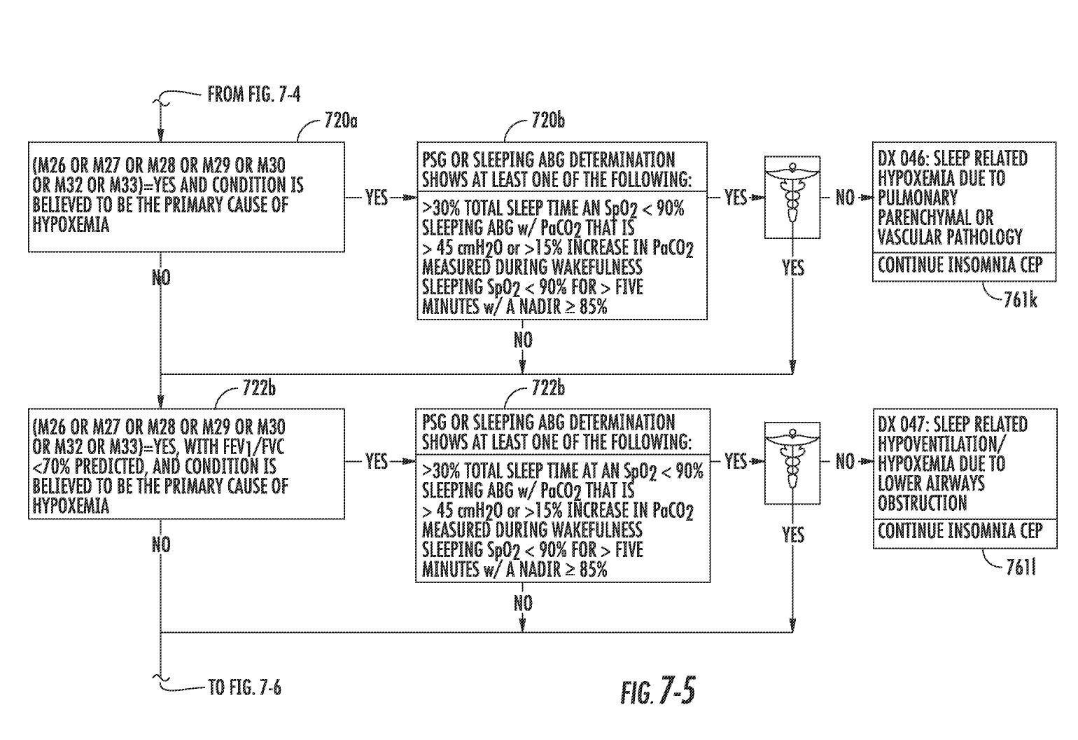

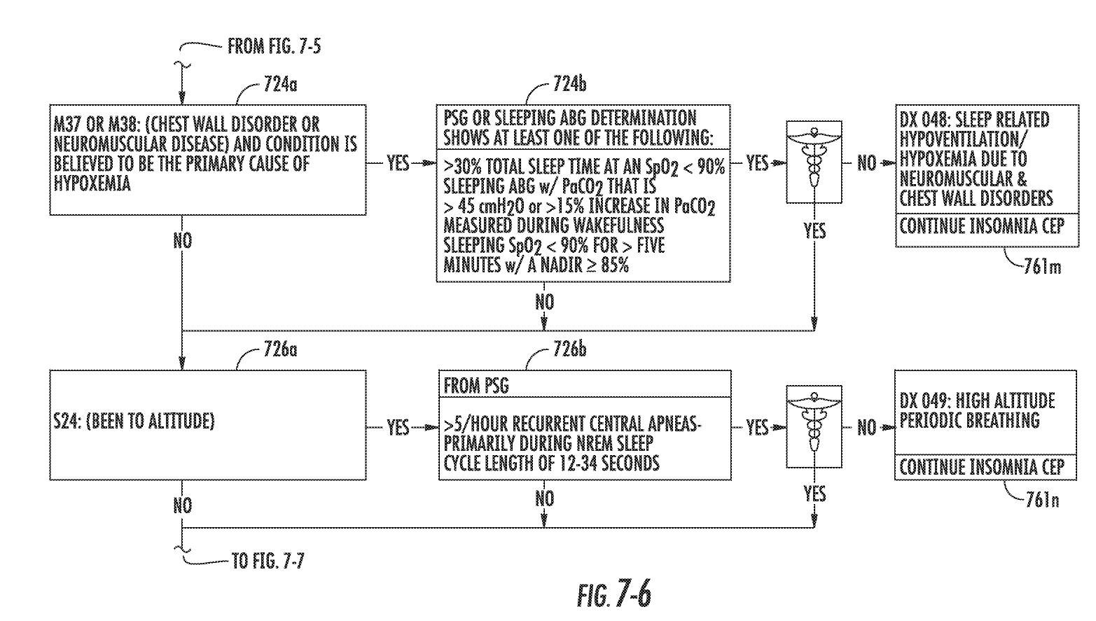

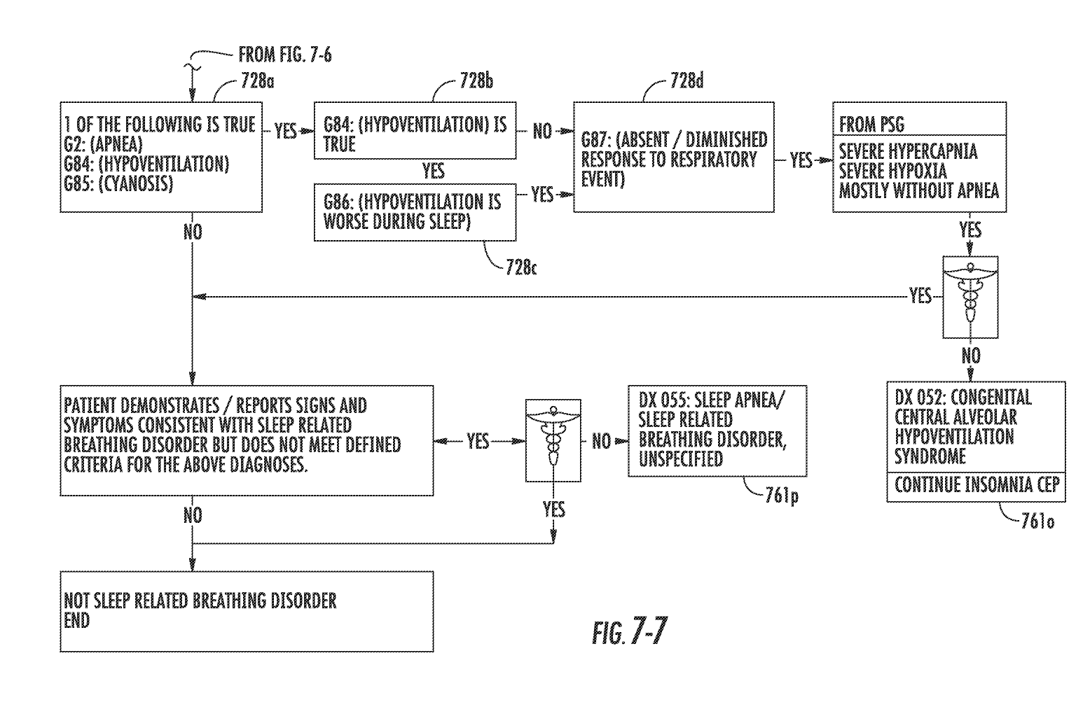

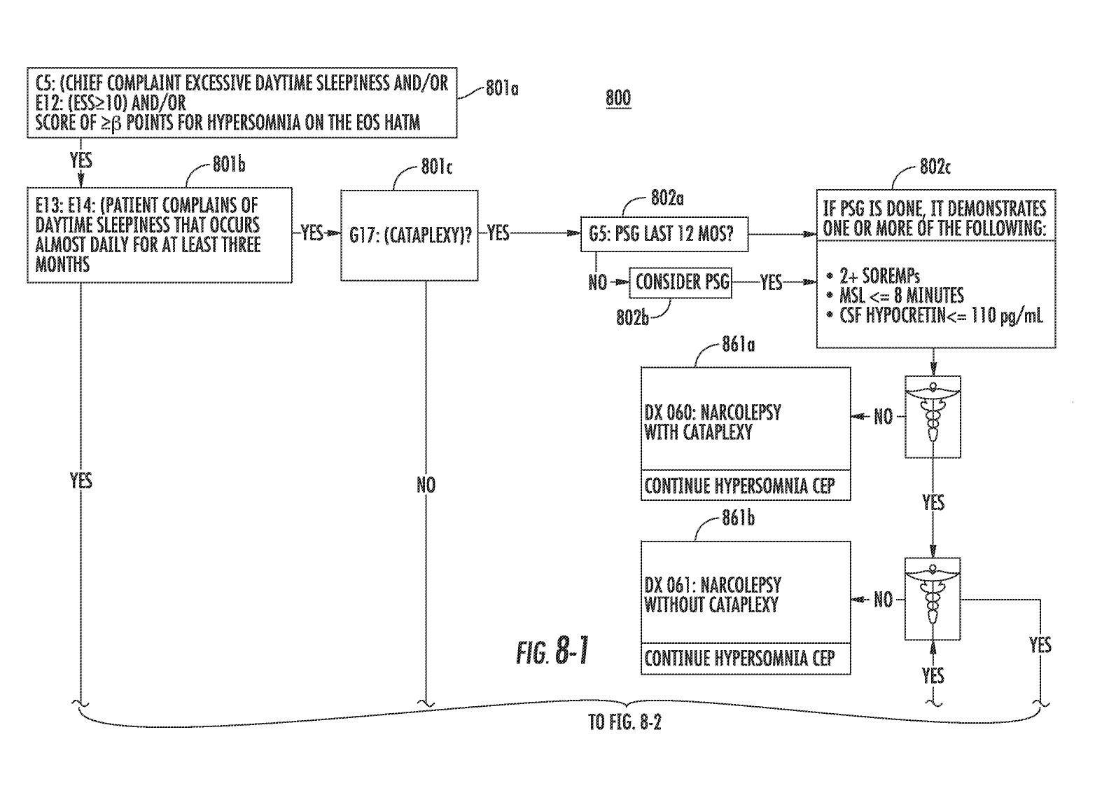

FIG. 7 illustrates a schematic diagram of breathing clinical evaluation pathway 700 from workflow 7 (WF7). Test follow-up block 701 is performed to evaluate breathing conditions. If the patient is ≧12 years old and has a score above a threshold on sleep apnea screen template 350, for example the threshold can be six, block 702, blocks 703a and 703b are performed to determine the score consisting of constructive or mixed apneas, hypopapneas or respiratory event related arousal (RERAS) for providing further diagnostic data collection for determining respective a diagnosis of mild obstructive sleep apnea adult 761a or a diagnosis of obstructive sleep apnea adult 761b. Alternatively, if the patient is less than 12 years old, blocks 704a-704h are performed before blocks 703a-703c. If the patient is ≧2 years old in block 706, blocks 707a-707c are performed for providing further diagnostic data collection for determining a diagnosis of obstructive sleep apnea pediatric 761c. If the patient is <2 years old, blocks 708a-708c are performed for providing respective further diagnostic data collection for determining a diagnosis of respective primary sleep apnea of infancy 761d or a diagnosis of primary apnea of infancy 761e.

In block 710 for a polysysmograph (PSG) if ≧5 central apnea/hour occur this further diagnostic data collection for determining a diagnosis of primary central sleep apnea 761f. In block 712a, for PSG if ≧10 apneas, hypopapneas, and hypoapneas have a crescendo/decrescendo respiratory pattern and are accompanied by frequent arousals and frequent sleep and serious medical illness determined in block 721b provide further diagnostic data collection for determining a diagnosis of Cheyne-Stokes breathing pattern 761g. Drug substance symptom block 714a and data for PSG block 714b provide further diagnostic data collection for determining a diagnosis of central sleep apnea due to drug or substance 761h.

Data for PSG of ≧ a threshold number of apneas and serious medical conditions determined in block 716b provide further diagnostic data collection for determining a diagnosis of central sleep apnea due to medical condition not Cheyne-Stokes 761i. Data of PSG or of hypopapneas in block 718a and ventilation conditions in block 719b provide further diagnostic data collection for determining a diagnosis of sleep related non-obstructive alveolar hypoventilation idiopathic 761j. Conditions block 720a and PSG information block 720b provide further diagnostic data collection for determining sleep related hypoxemia due to pulmonary parenthymal or vascular pathology diagnosis 761k. Conditions block 722a and PSG information block 722b provide further diagnostic data collection for determining a diagnosis of sleep related hypoventilation/hypoxemia due to lower airways obstruction 761l. Conditions block 724a and PSG information block 724b provide further diagnostic data collection for determining a diagnosis of sleep related hypoventilation/hypoexmia due to neuromuscular and chest wall disorders 761m. Conditions block 726a and PSG information block 726b provide further diagnostic data collection for determining a diagnosis of high-altitude periodic breathing 761n. Conditions of block 728a-728d and PSG information block 728e provide further diagnostic data collection for determining a diagnosis of congenital central alveolar hypoventilation syndrome 761o. Other symptoms block 730 provides further diagnostic data collection for determining a diagnosis of sleep apnea/sleep related breathing disorder unspecified 761p.

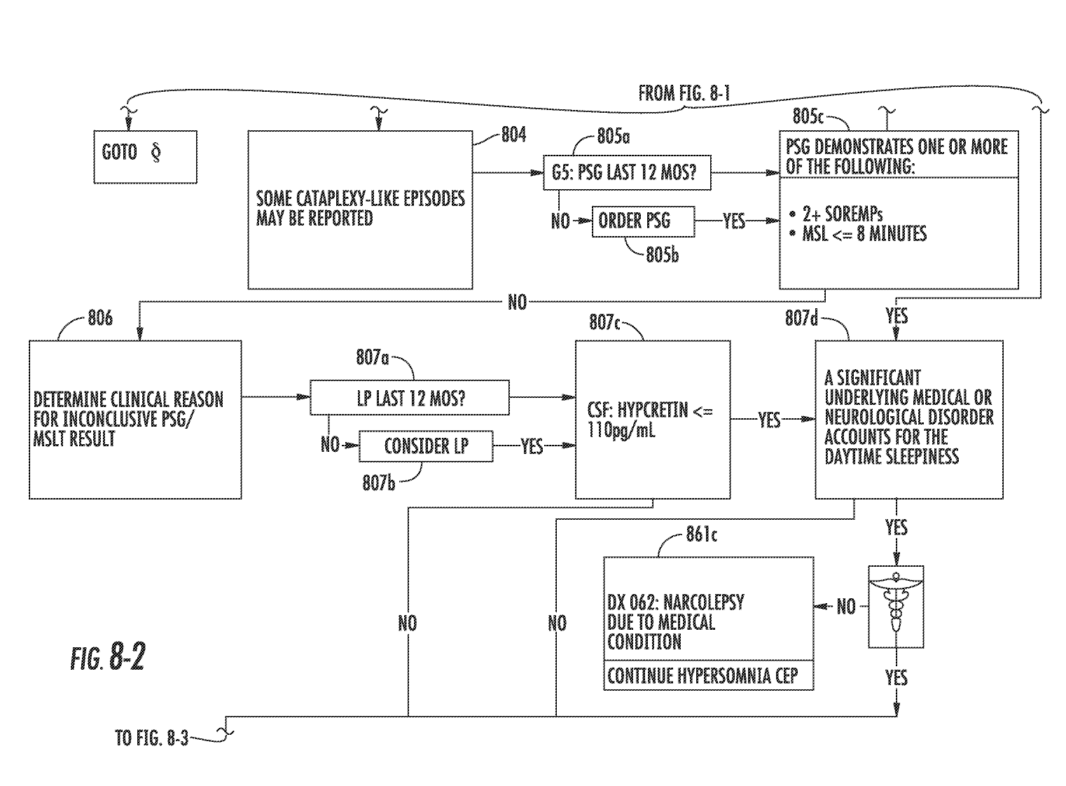

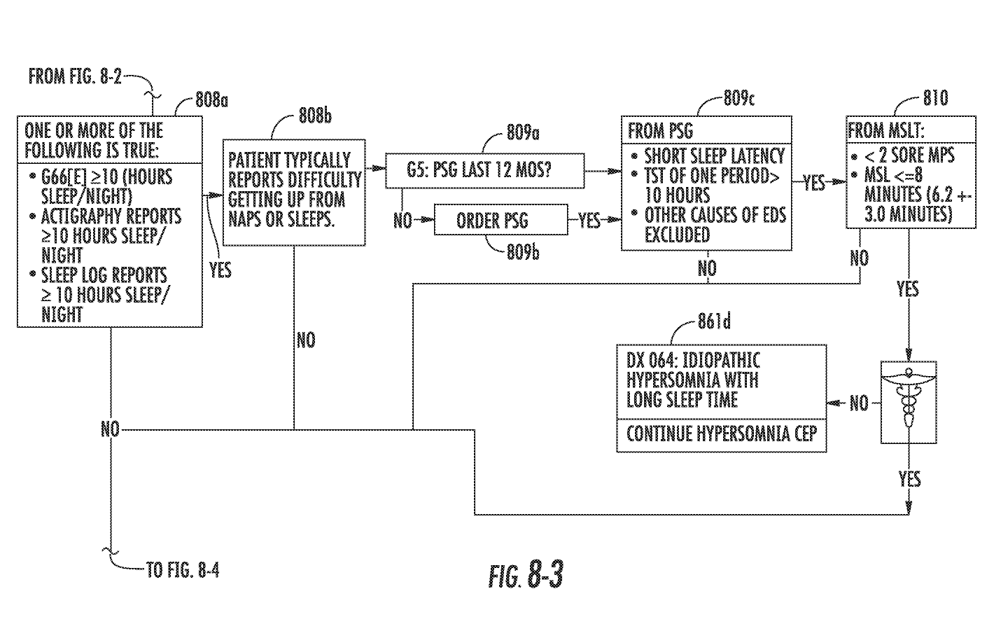

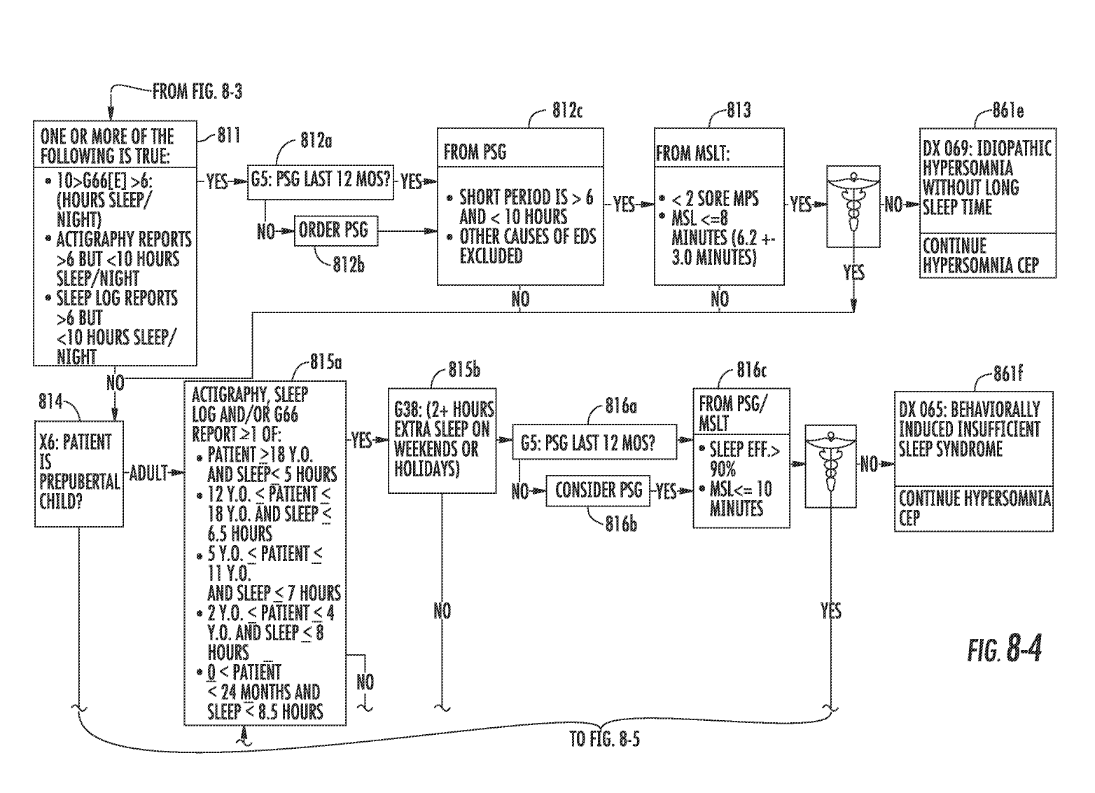

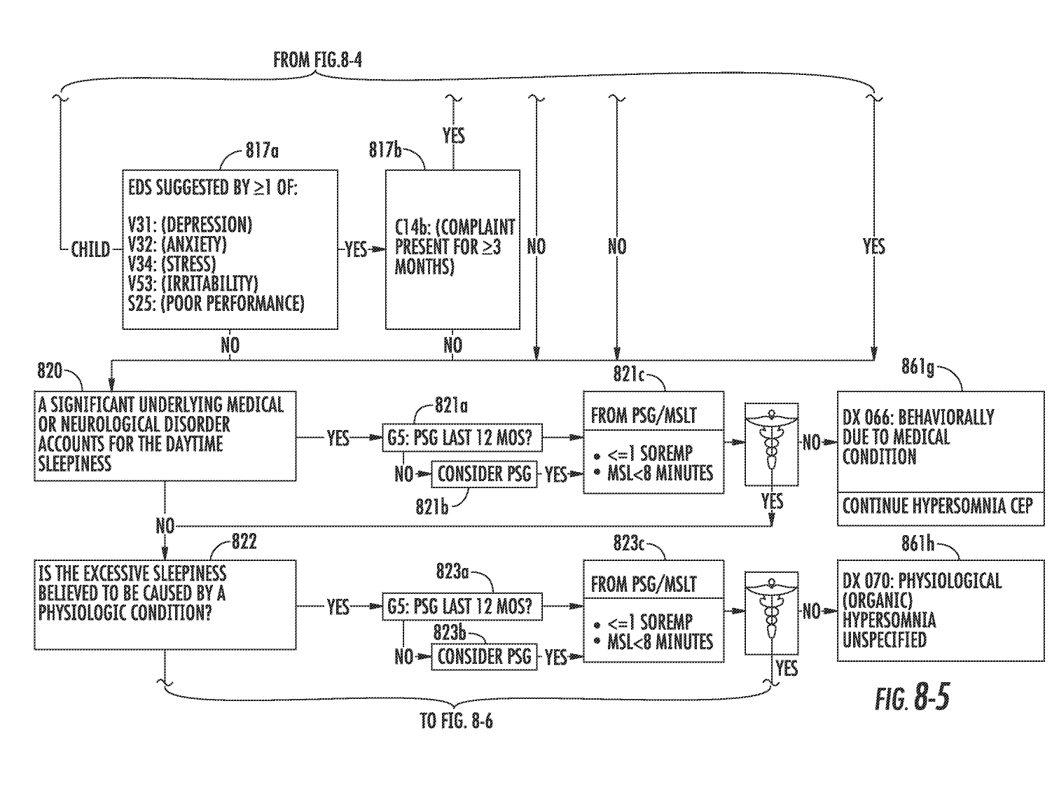

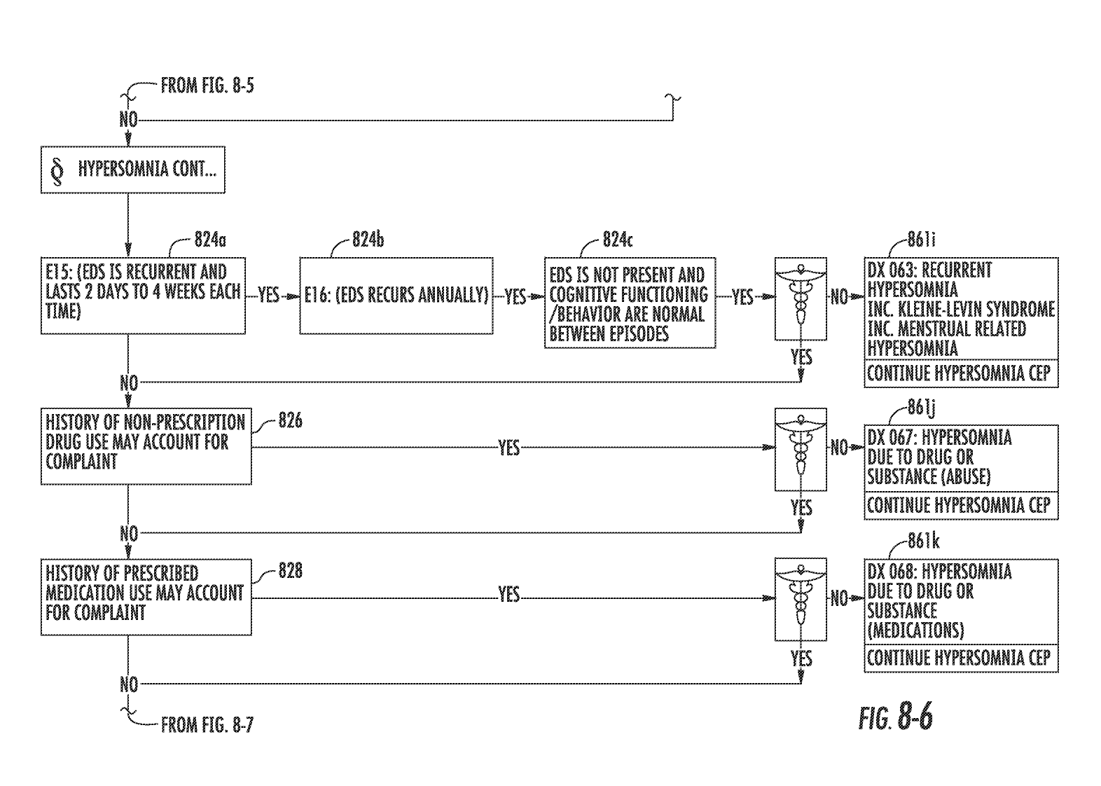

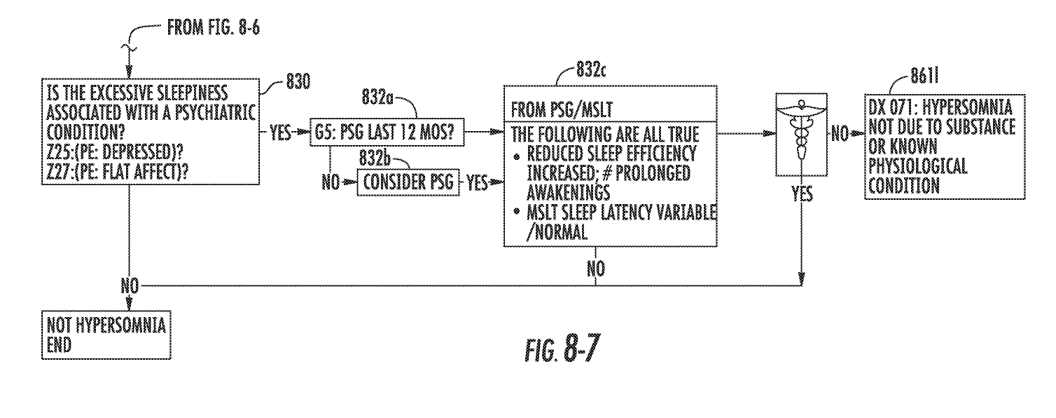

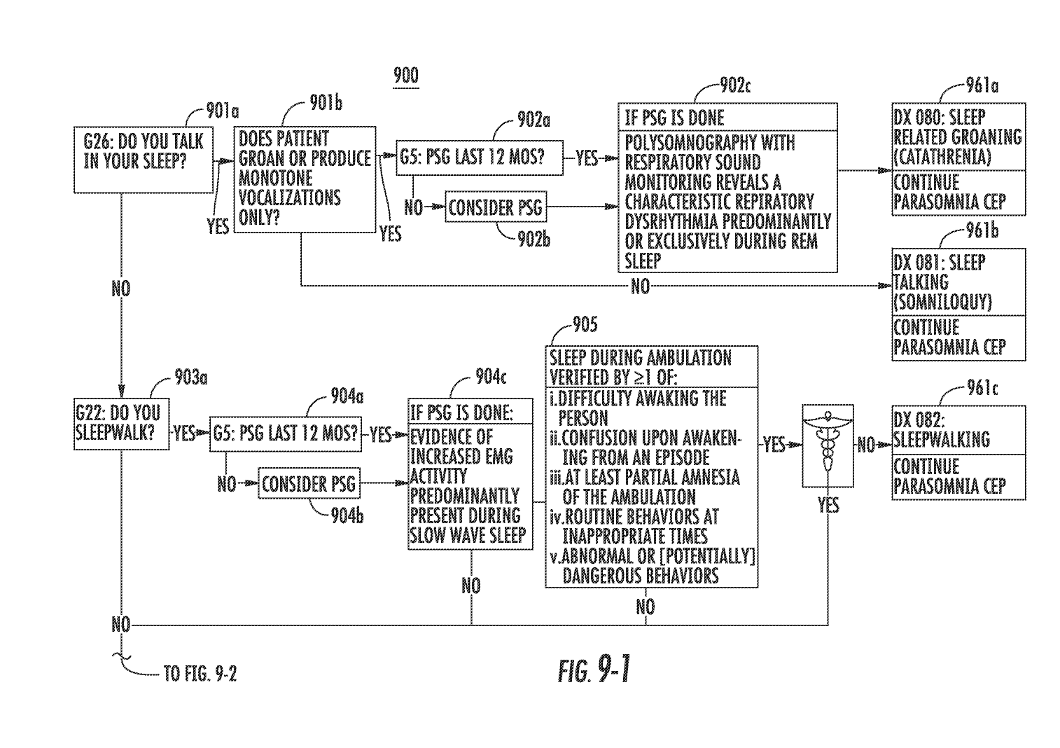

FIG. 8 illustrates a schematic diagram of hypersomnia clinical evaluation pathway from workflow 8 (WF8). Condition data blocks 801a-801c and PSG data blocks 802a-802c provide further diagnostic data collection for determining a diagnosis of narcolepsy with cataplexy 861a. Condition data block 804 and PSG data blocks 805a-805c provide further diagnostic data collection for determining a diagnosis of narcolepsy without cataplexy 861b. Block 806 deter mines a clinical reason for an inclusive PSG or multiple sleep latency test (MSLT) result. Blocks 807a-807c determines consideration for a lumbar puncture (LP) and block 807d determines medical condition data providing further diagnostic data collection for determining a diagnosis of narcolepsy due to medical condition 861c. Conditions 808a and 808b, PSG data blocks 809a-809c and MSLT data block 810 provide further diagnostic data collection for determining a diagnosis of idiopathic hypersomnia with long sleep time 861d. Conditions 811, PSG data blocks 812a-812c and MSLT data 813 provide further diagnostic data collection for determining a diagnosis of idiopathic hypersomnia without long sleep time 861e. For a child, blocks 817a and 817b are conditions determined before block 815a. Block 814 determines if the patient is an adult or a child. Block 815a determines sleep log conditions and extra sleep conditions 815b and PSG blocks 816a-816c provide further diagnostic data collection for determining a diagnosis of behaviorally induced insufficient sleep syndrome 861f.

Block 820 provides data on a significant underlying medical neurological disorder accounting for daytime sleepiness and PSG data blocks 821a-821c provide further diagnostic data collection for determining a diagnosis of hypersomnia due to medical condition 861g. Block 822 provides data on sleepiness caused by physiological conditions and PSG data blocks 821a-821c provide further diagnostic data collection for determining a diagnosis of physiological (organic) hypersomnia unspecified 861h. Blocks 824a-824c determine excessive daytime sleepiness (EDS) data for providing further diagnostic data collection for determining a diagnosis of recurrent hypersomnia 861i. Block 826 provides data for history of non-prescription drugs and provides further diagnostic data collection for determining a diagnosis of hypersomnia due to drug or substance (abuse) 861j. Block 828 provides data for prescribed medication and provides further diagnostic data collection for determining a diagnosis of hypersomnia due to drug or substance (medication) 861k. Condition block 830 and PSG data blocks 832a-832c provide further diagnostic data collection for determining a diagnosis of hypersomnia not due to substance or known physiological conditions 861l.

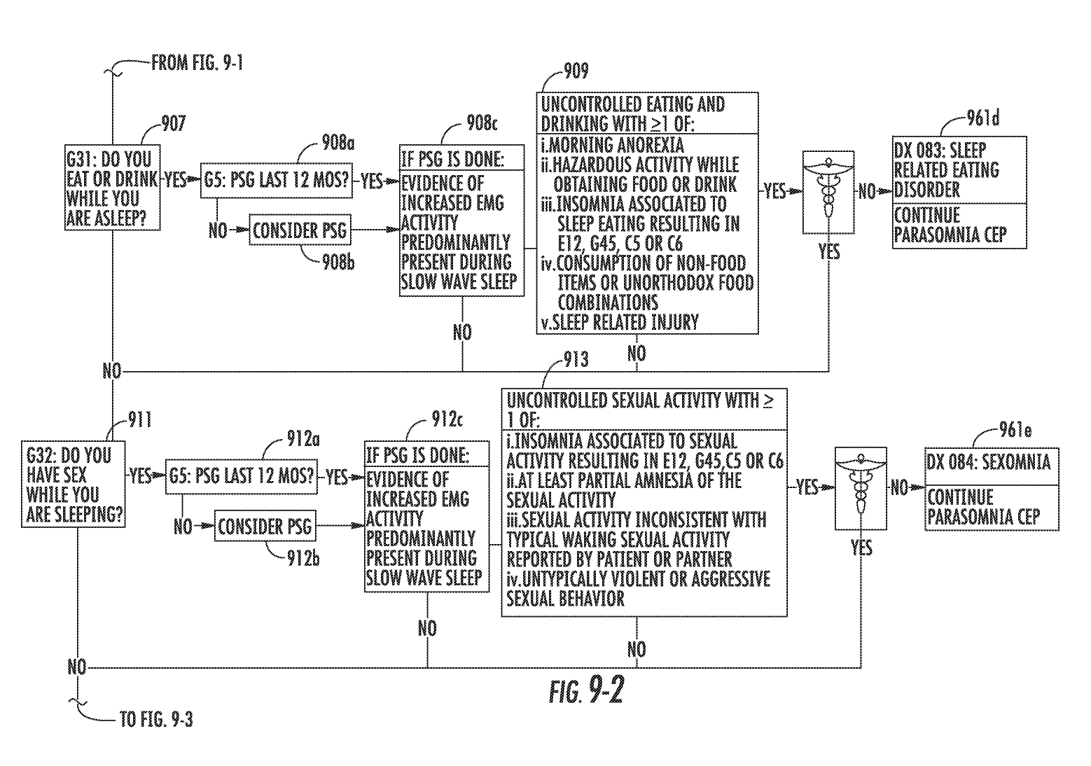

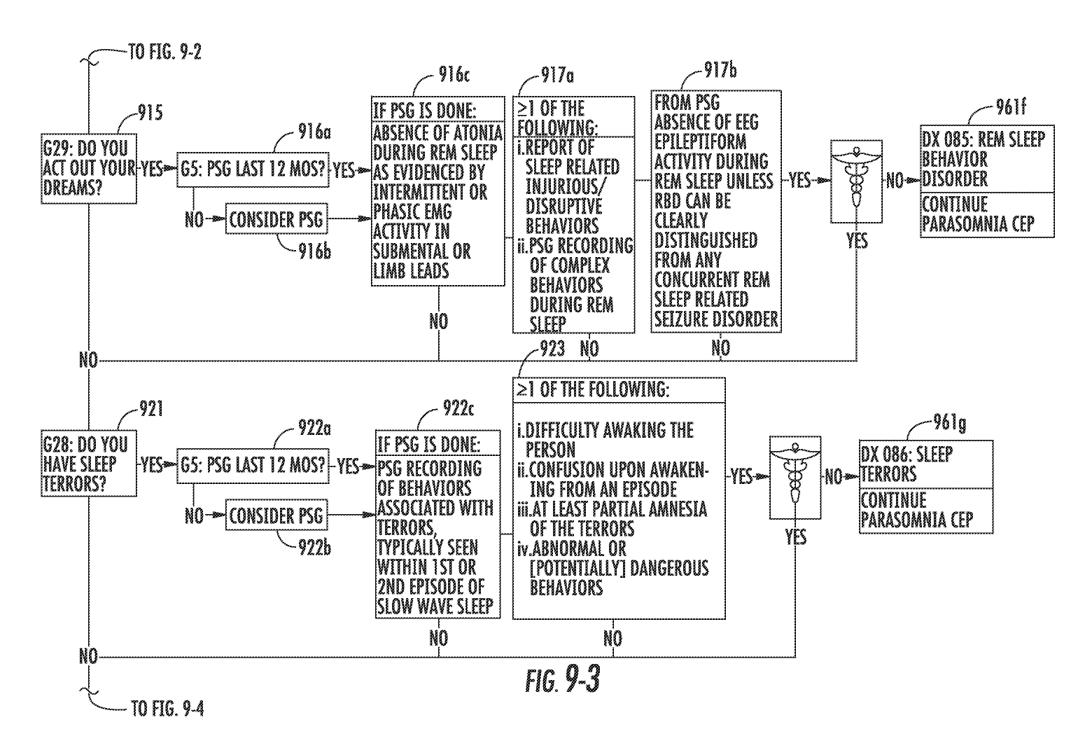

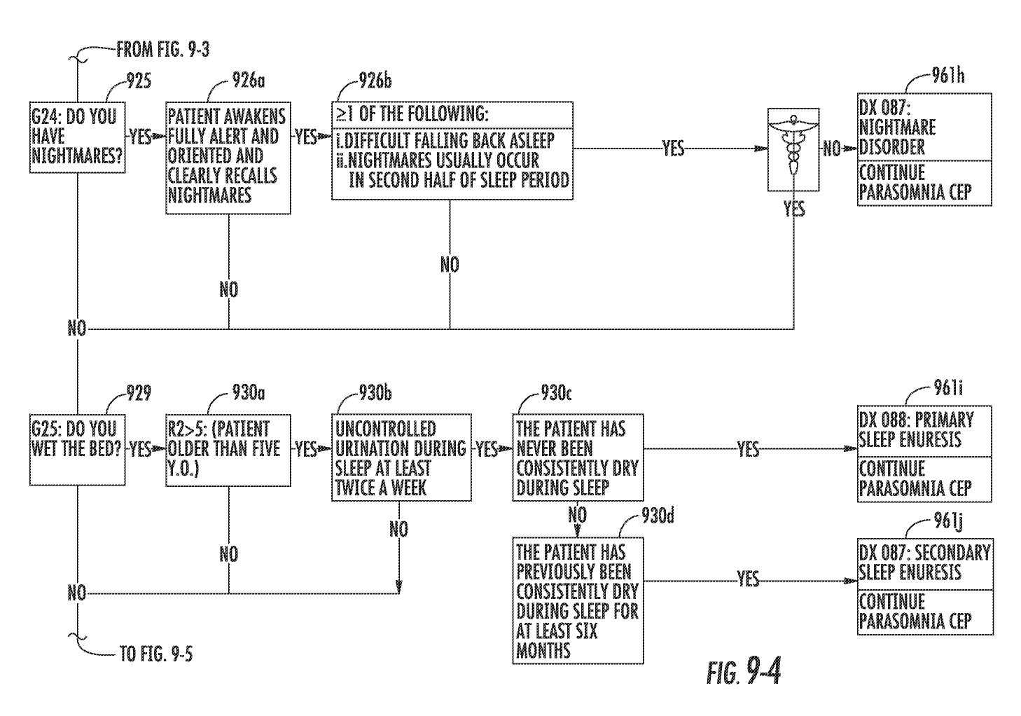

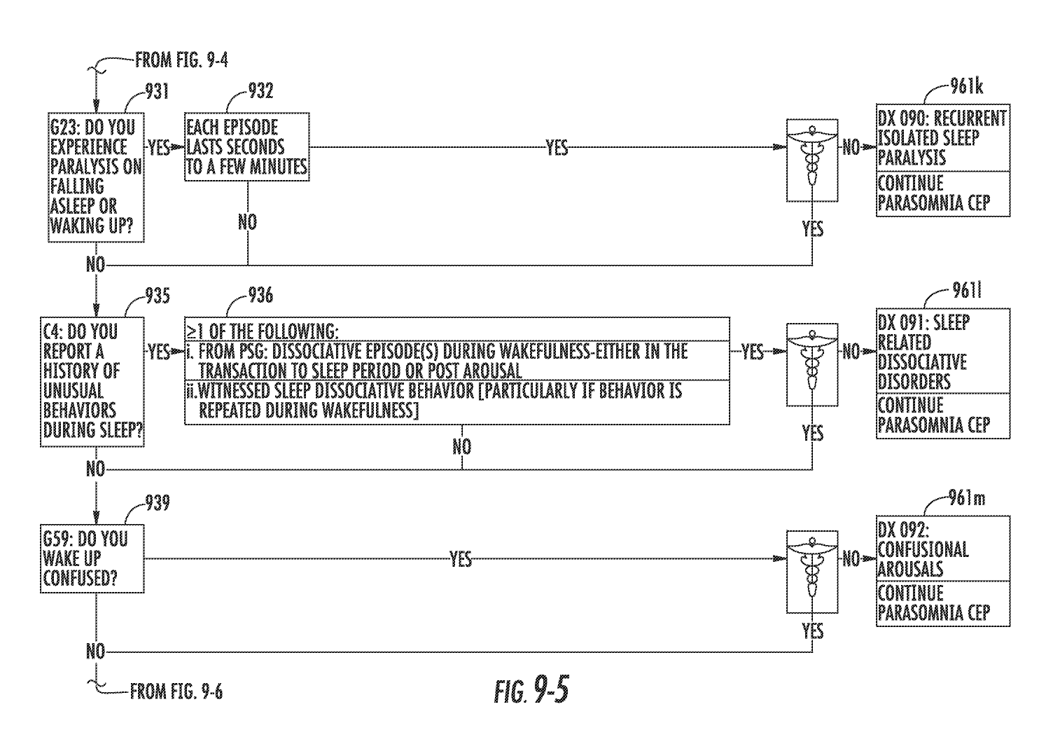

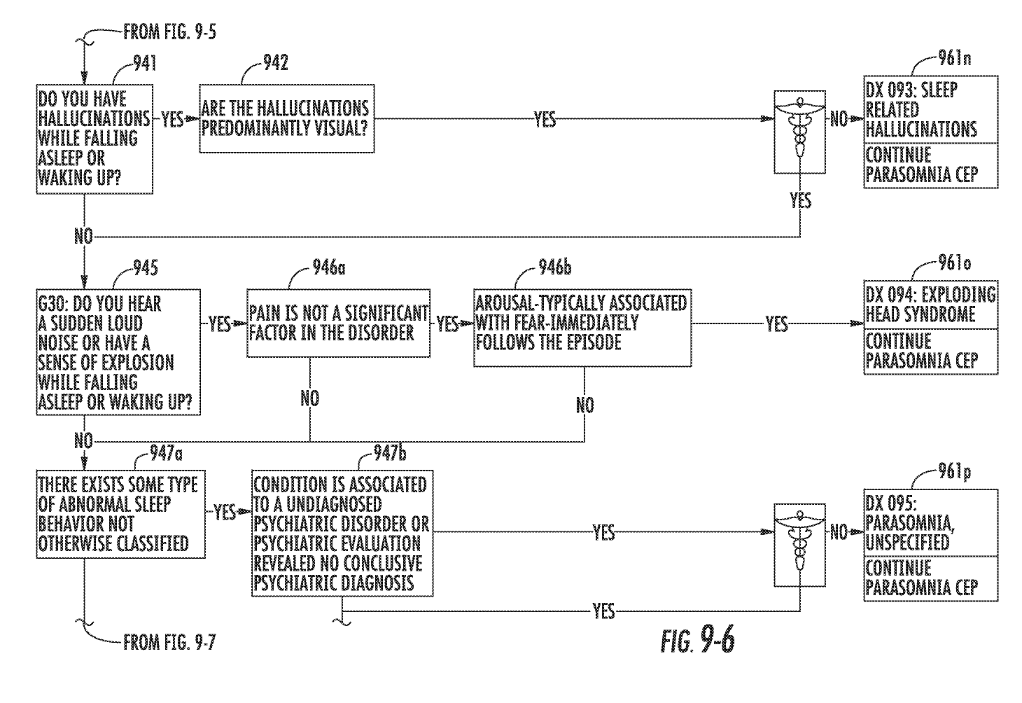

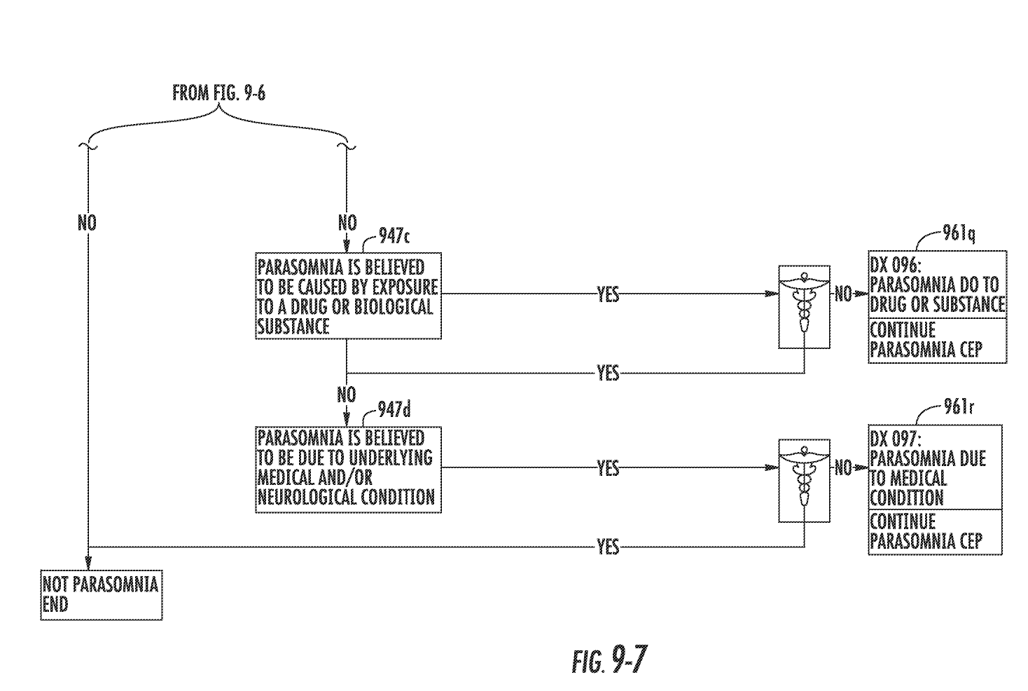

FIG. 9 illustrates parasomnia clinical evaluation pathway 900 from workflow 9 (WF9). Questions 901a and 901b and PSG data blocks 902a-902c provide further diagnostic data collection for determining a diagnosis of sleep related groaning (catathrenia) 961a or a diagnosis of sleep talking (somnlioquy) 961b. Questions 903a, PSG data blocks 904a-904c and conditions verifying ambulation block 905 provide further diagnostic data collection for determining a diagnosis of sleepwalking 961c. Question 907, PSG data blocks 908a-908c and conditions verifying uncontrolled eating and drinking 909 provide further diagnostic data collection for determining a diagnosis of sleep related eating disorder 961d. Question 911, PSG data blocks 912a-912c and conditions verifying uncontrolled sexual activity 913 provide further diagnostic data collection for determining a diagnosis of sexsomnia 961e. Question 915c, PSG data blocks 916a-916c and conditions 917a and 917b provide further diagnostic data collection for determining a diagnosis of REM sleep behavioral disorder 961f. Questions 921, PSG data blocks 922a-922c and conditions on awakening provide further diagnostic data collection for determining a diagnosis of sleep terrors 961g. Question 925 and conditions 926a and 926b provide further diagnostic data collection for determining a diagnosis of nightmare disorder 961h. Question 929 and conditions 930a-930d provide further diagnostic data collection for determining a respective diagnosis of primary sleep enuresis 961i and a diagnosis of secondary sleep enuresis 961j. Question 931 and condition 932 provide further diagnostic data collection for determining a diagnosis of recurrent isolated sleep paralysis 961k. Question 935 and conditions 936 provide further diagnostic data collection for determining a diagnosis of sleep related dissociative disorder 961l. Question 939 provides further diagnostic data collection for determining a diagnosis of confusional arousals 961m. Question 941 and condition 942 provide further diagnostic data collection for determining a diagnosis of sleep related hallucinations 961n. Question 945 and conditions 946a and 946b provide further diagnostic data collection for determining a diagnosis of exploding head syndrome 961o. Conditions 947a-947d determine further diagnostic data collection for determining a respective diagnosis of parasomnia unspecified 961p, a diagnosis of parasomnia due to drug or substance 961q and a diagnosis of parasomnia due to medical condition 961r.

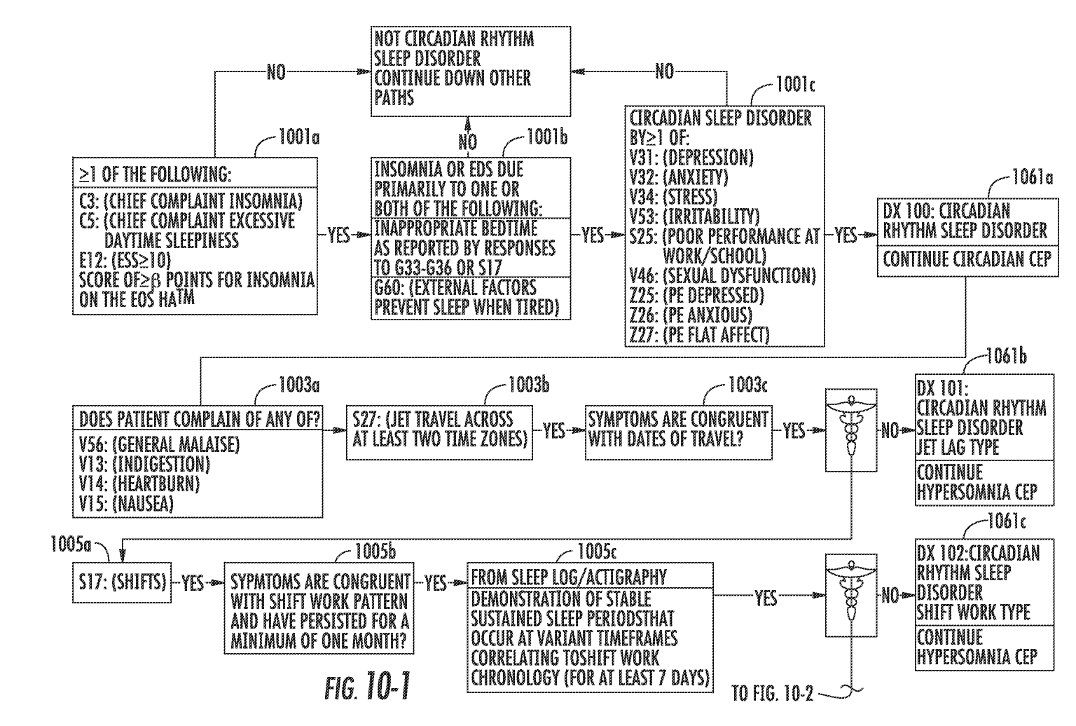

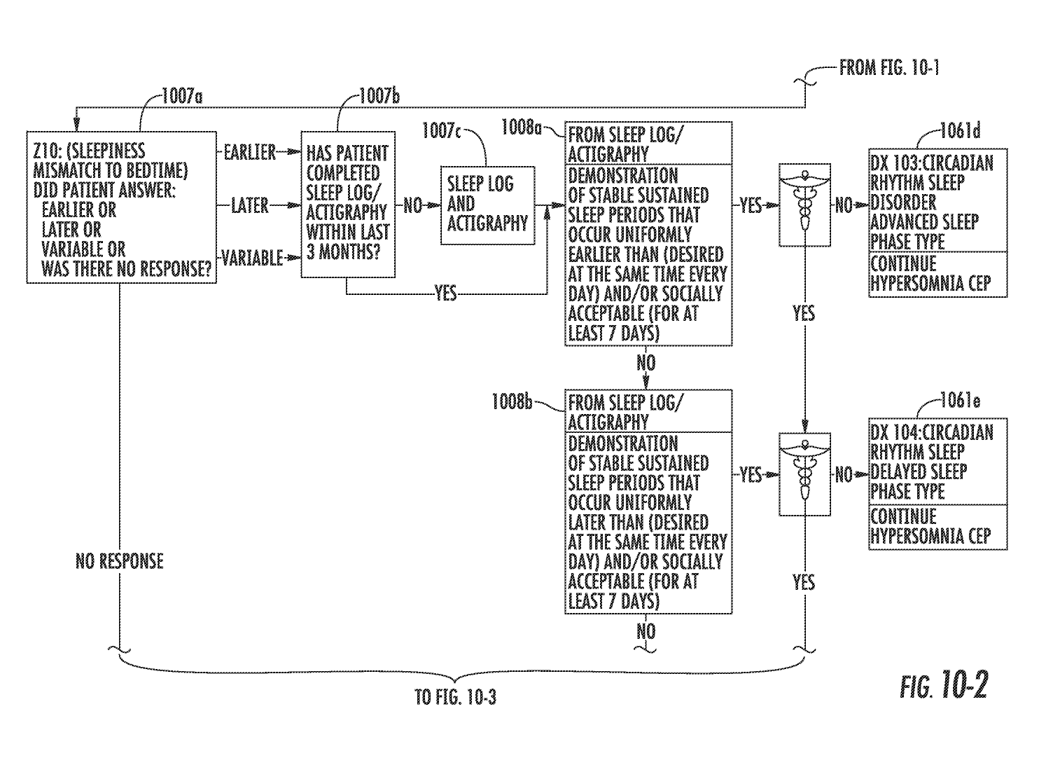

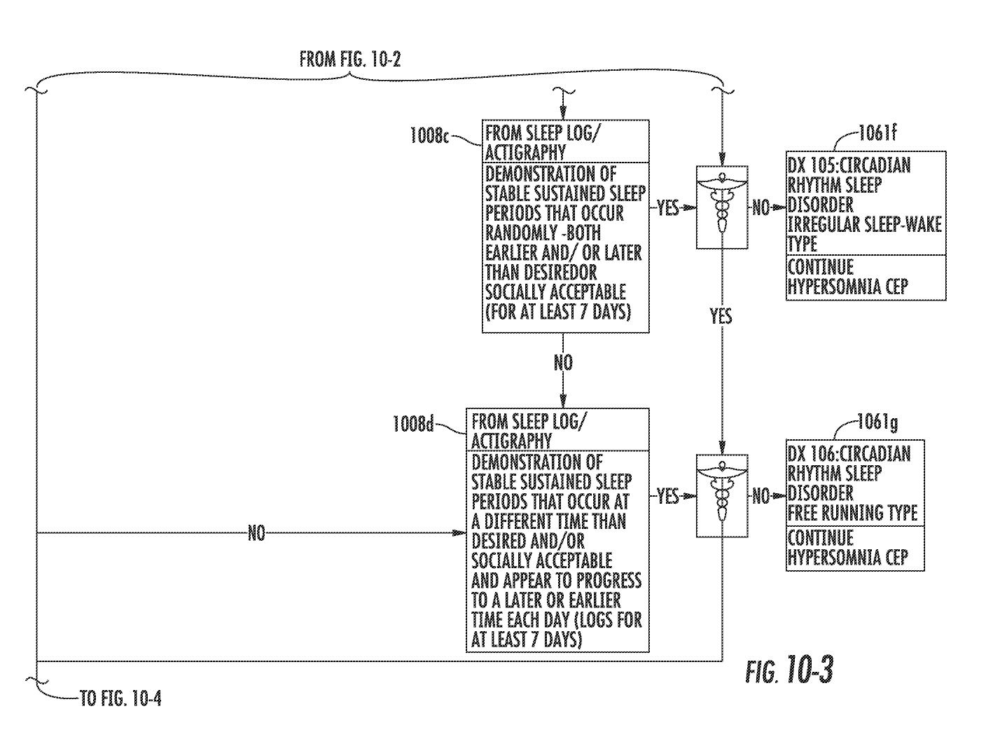

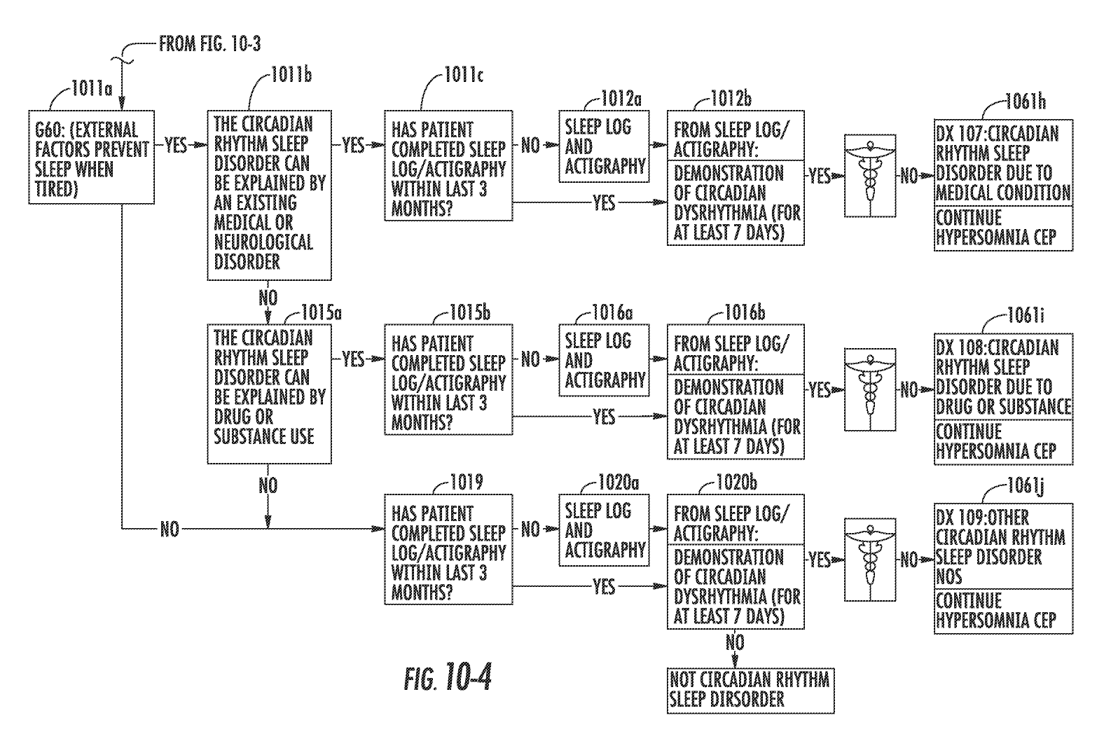

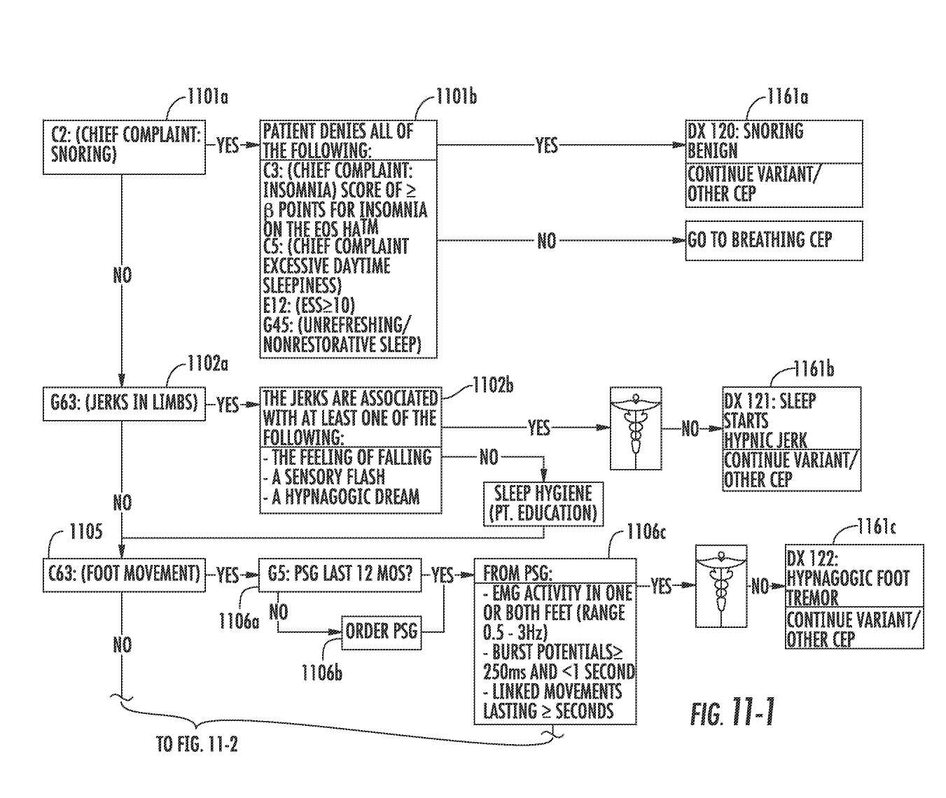

FIG. 10 illustrates a schematic diagram of a circadian clinical evaluation pathway 1000 from workflow 10 (WF10). Conditions 1001a-1001e provide further diagnostic data collection for determining a diagnosis of circadian rhythm sleep disorder 1061a. Conditions 1003a-1003c provide further diagnostic data collection for determining a diagnosis of circadian rhythm sleep disorder (jet lag type) 1061b. Conditions 1005a-1005c provide further diagnostic data collection for determining a diagnosis of circadian rhythm sleep disorder (shift work type) 1061c. Conditions 1007a-1007c and sleep log actigraph 1008a-1008d provide further diagnostic data collection for determining a respective diagnosis of circadian rhythm sleep disorder (advanced sleep phase type) 1061d; a diagnosis of circadian rhythm sleep disorder (delayed sleep phase type) 1061e; a diagnosis of circadian rhythm sleep disorder (irregular sleep phase type) 1061f; and a diagnosis of circadian rhythm sleep disorder (free running type) 1061g.

Conditions 1011a-1011c and sleep log actigraph 1012a and 1012b provide further diagnostic data collection for determining a diagnosis of circadian rhythm sleep disorder (due to medical condition) 1061h. Conditions 1015a and 1015b and sleep log actigraph 1016 provide further diagnostic data collection for determining a diagnosis of circadian rhythm sleep disorder (due to drug or substance abuse) 1061i. Condition 1019 and sleep log actigraph 1020a and 1020b provide further diagnostic data collection for determining a diagnosis of circadian rhythm sleep disorder (NOS) 1061j.

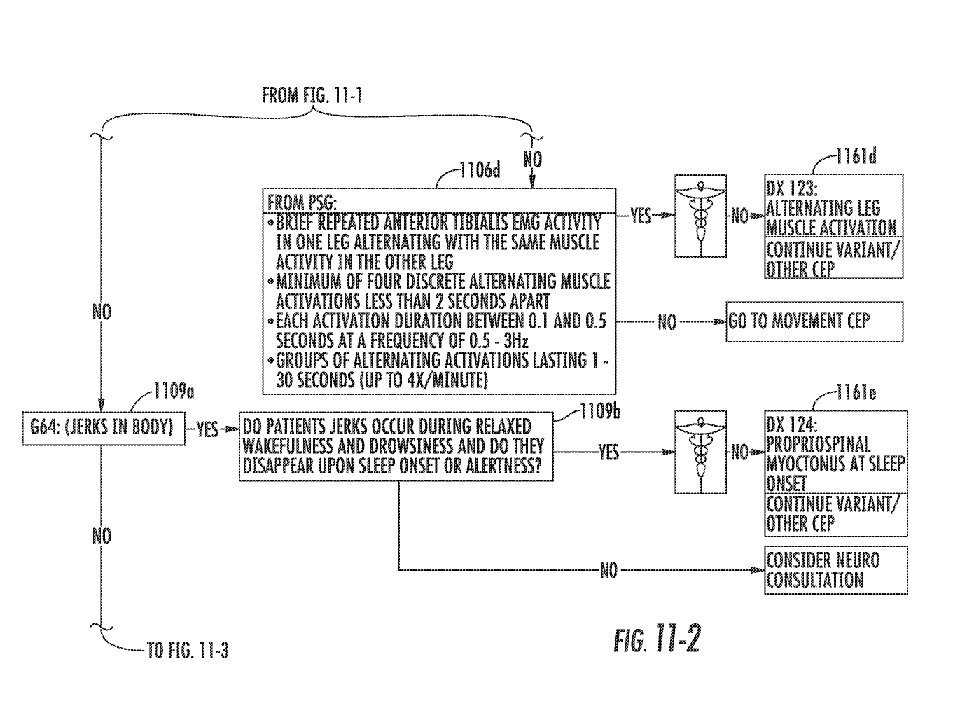

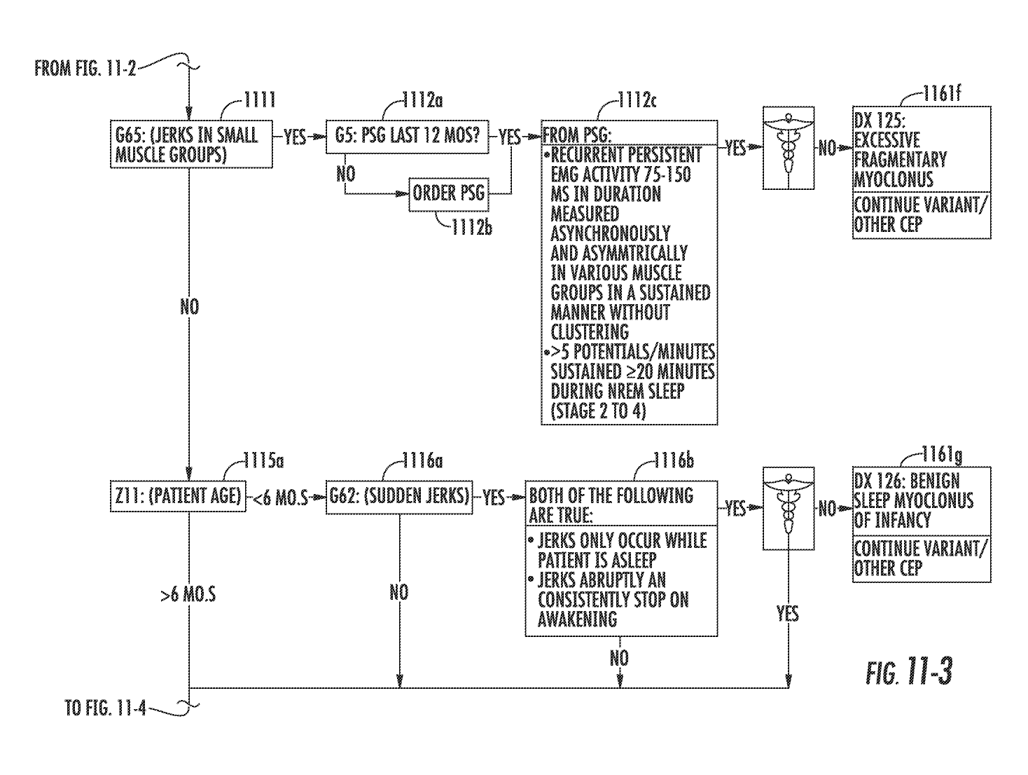

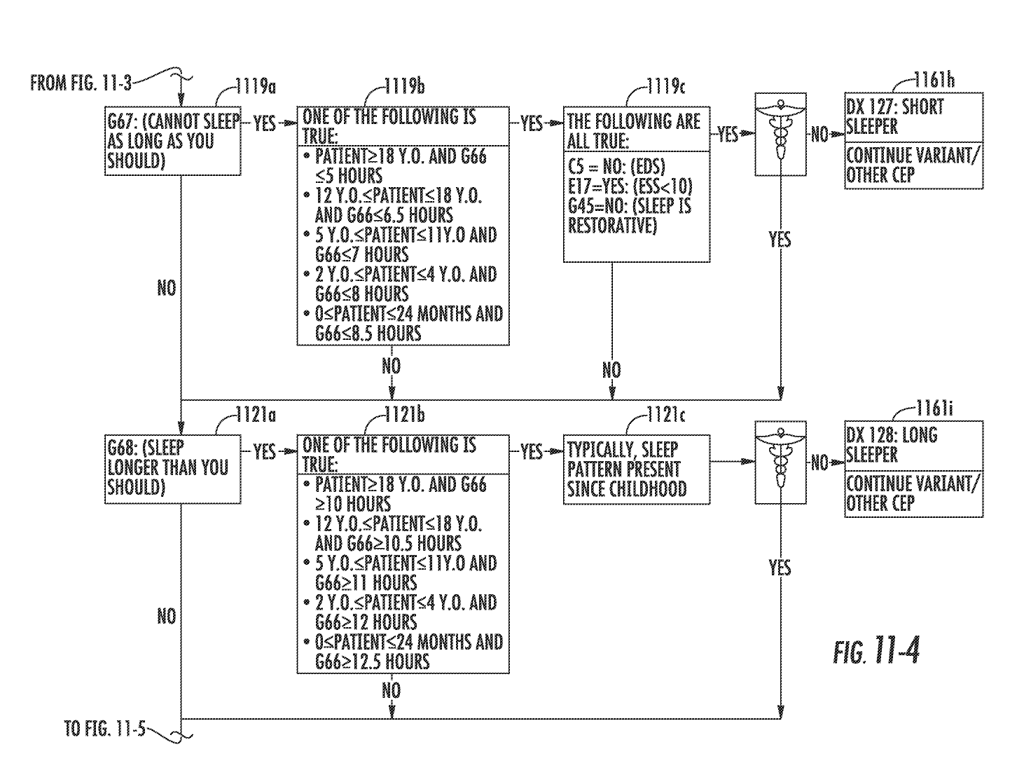

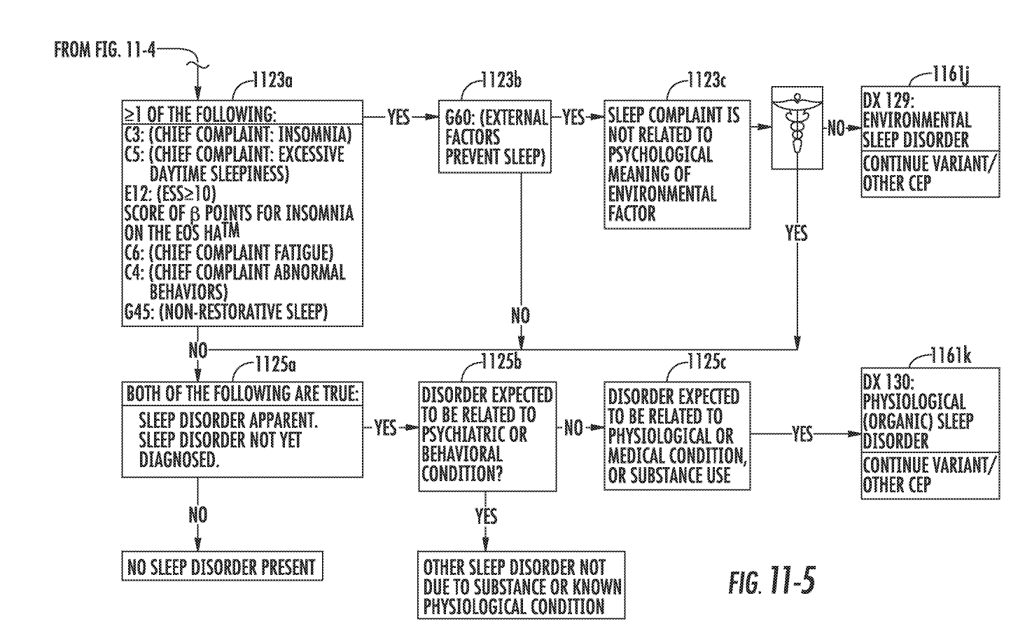

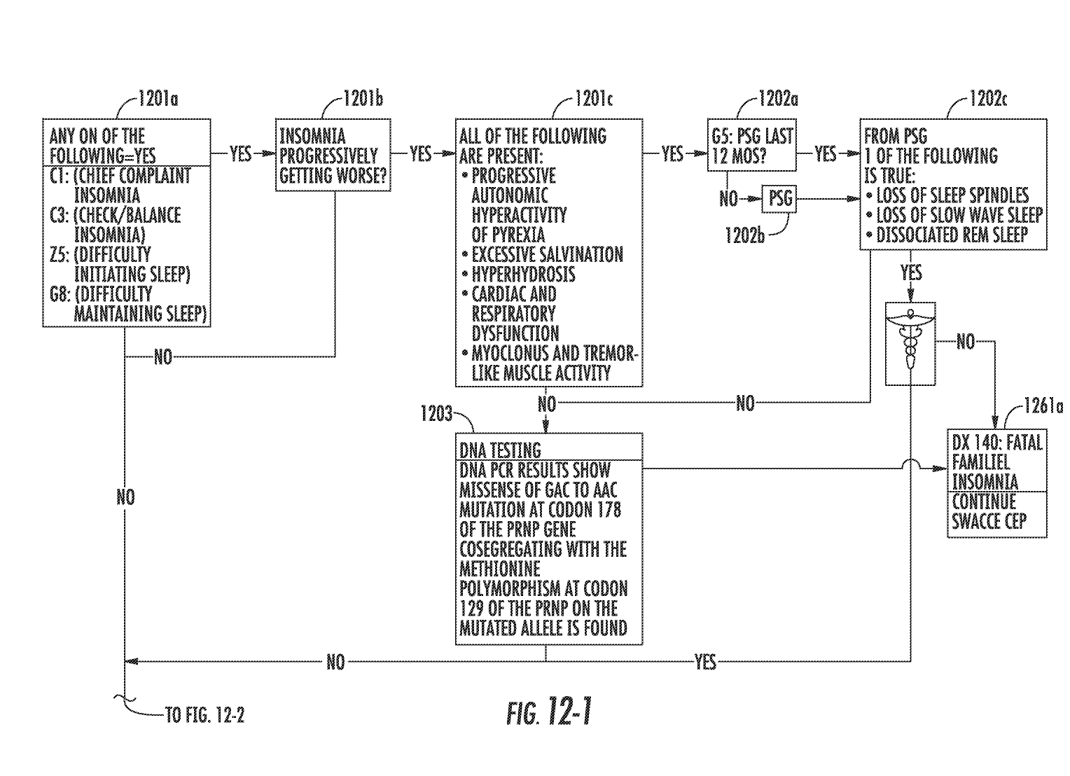

FIG. 11 illustrates variant clinical evaluation pathway 1100 from workflow 11 (WF11). Conditions 1101a and 1101b provide further diagnostic data collection for determining a diagnosis of snoring (benign) 1161a. Conditions 1102a-1102c provide further diagnostic data collection for determining a diagnosis of sleep starts (hypnic jerks) 1161b. Conditions 1105 and PSG data blocks 1106a-1106d provide further diagnostic data collection for determining a respective diagnosis of hypnagogic foot tremor 1161c and a diagnosis of alternating leg muscle activation 1161d. Conditions 1109a and 1109b provide further diagnostic data collection for determining a diagnosis of propriospinal myocionus at sleep onset 1161e. Condition 1111 and PSG data blocks 112a-112c provide further diagnostic data collection for determining a diagnosis of excessive fragmentary myocionus 1161f. Patient age condition of less than six months and conditions 1116a-1116d provide further diagnostic data collection for determining a diagnosis of benign sleep myocionus of infancy 1161g. Patient age condition of greater than six months and conditions 1119a-1119c provide further diagnostic data collection for determining a diagnosis of short sleeper 1161h. Conditions 1121a-1121c provide further diagnostic data collection for determining a diagnosis of long sleeper 1161i. Conditions 1123a-1123c provide further diagnostic data collection for determining a diagnosis of environmental sleep disorder 1161j. Conditions 1125a-1125c provide further diagnostic data collection for determining a diagnosis of other physiological (organic) sleep disorder 1161k.

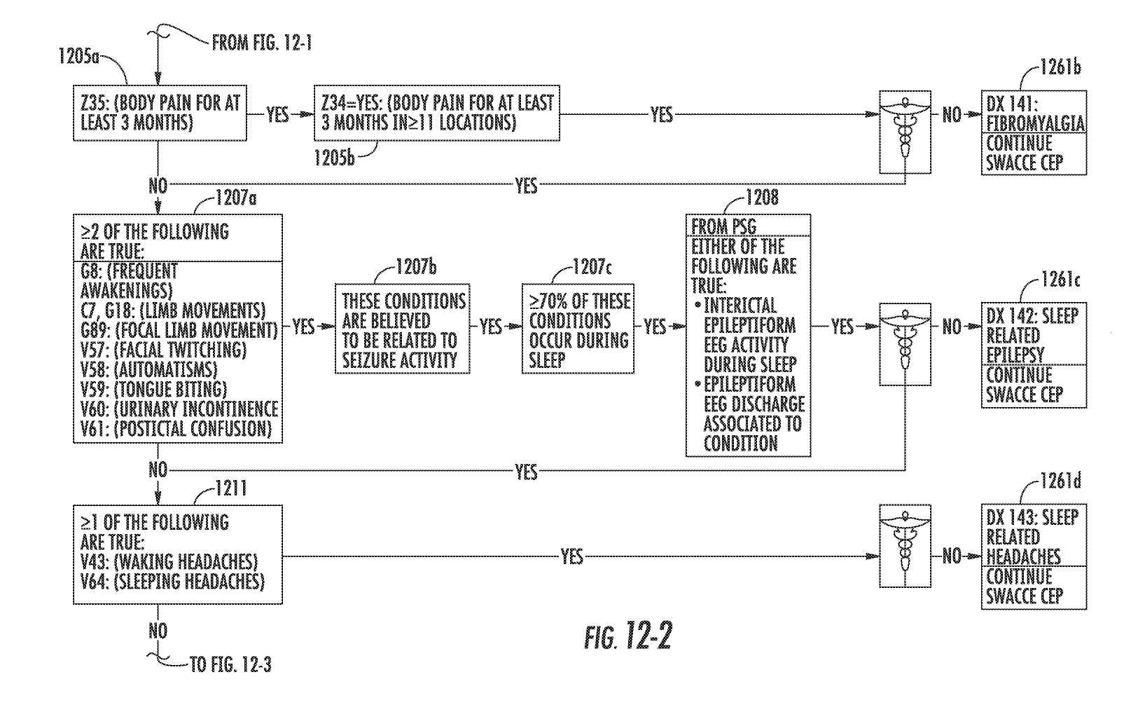

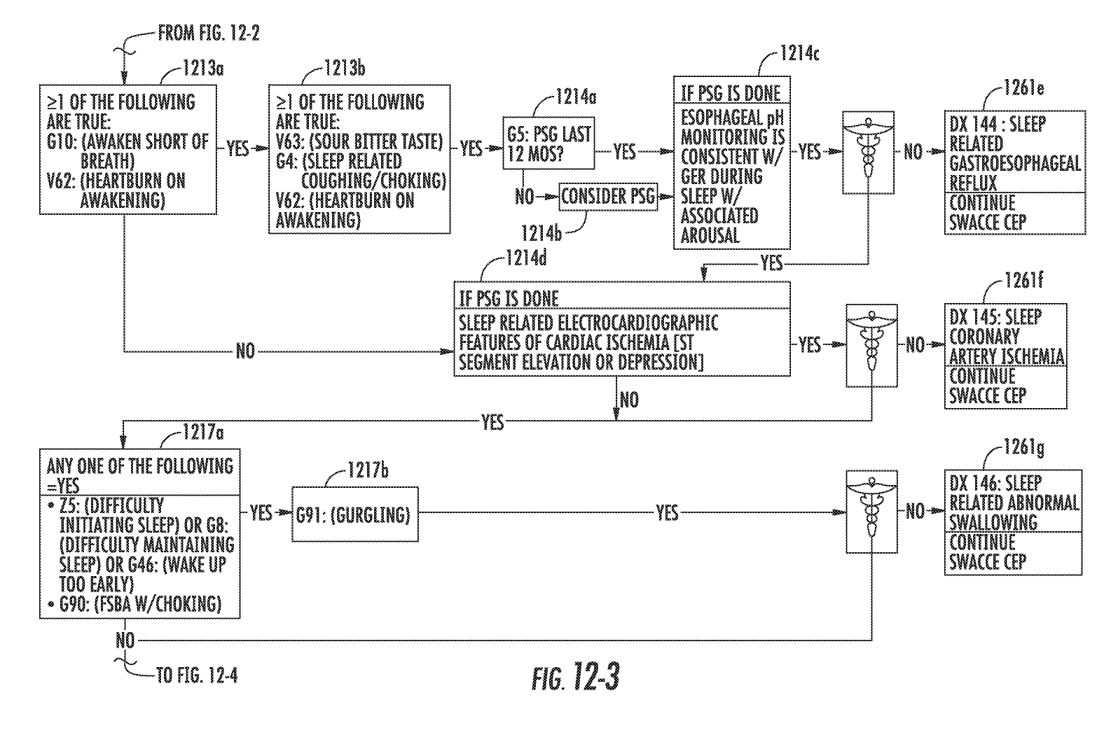

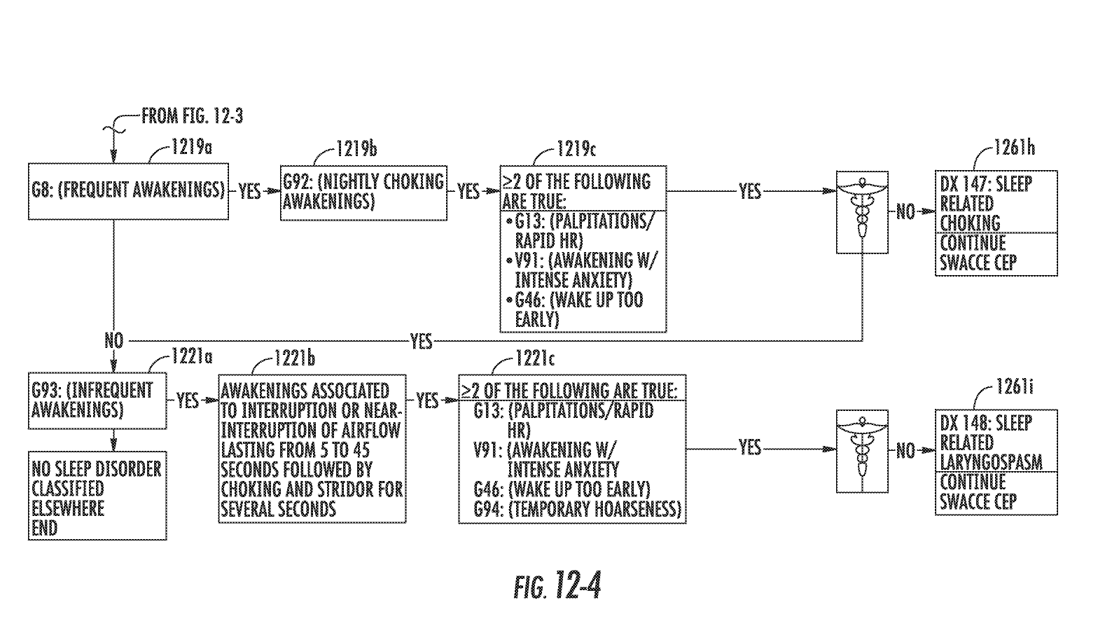

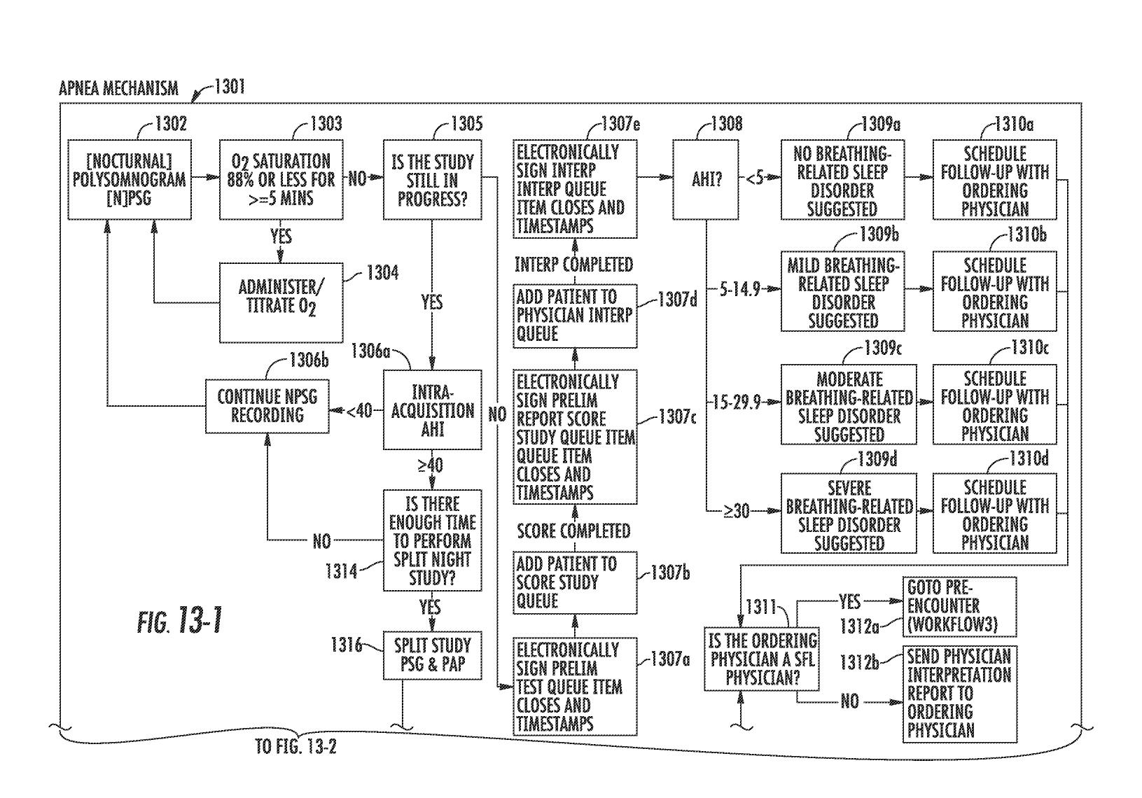

FIG. 12 illustrates sleep disorders related to other conditions clinical evaluation pathway 1200 from workflow 12 (WF12). Conditions 1201a-1201c, PSG data blocks 1202a-1202c and DNA testing data 1203 provide further diagnostic data collection for determining a diagnosis of fatal familial insomnia 1261a. Example DNA testing data can include DNA-PCR results show findings of missense of GAC to AAC mutation at codon 178 of the PRNP gene cosegregating with the methionine polymorphism at codon 129 of the PRNP on the mutated allele. Conditions 1205a and 1205 provide further diagnostic data collection for determining a diagnosis of fibromyalgia 1261b. Conditions 1207-1207c and PSG data blocks 1208 provide further diagnostic data collection for determining a diagnosis of sleep related epilepsy 1261c. Conditions 1211 provide further diagnostic data collection for determining a diagnosis of sleep related headaches 1261d. Conditions 1213a and 1213b and PSG data blocks 1214a-1214d respectively provide further diagnostic data collection for determining a respective diagnosis of sleep related gastroesophageal reflux 1261e and a diagnosis of sleep related coronary artery ischemia 1261f. Conditions 1217a and 1217b provide further diagnostic data collection for determining a diagnosis of sleep related abnormal 1261g. Conditions 1219a-1219c provide further diagnostic data collection for determining a diagnosis of sleep related choking 1261h. Conditions 1221a-1221c provide further diagnostic data collection for determining a diagnosis of sleep related laryngospasm 1261i.

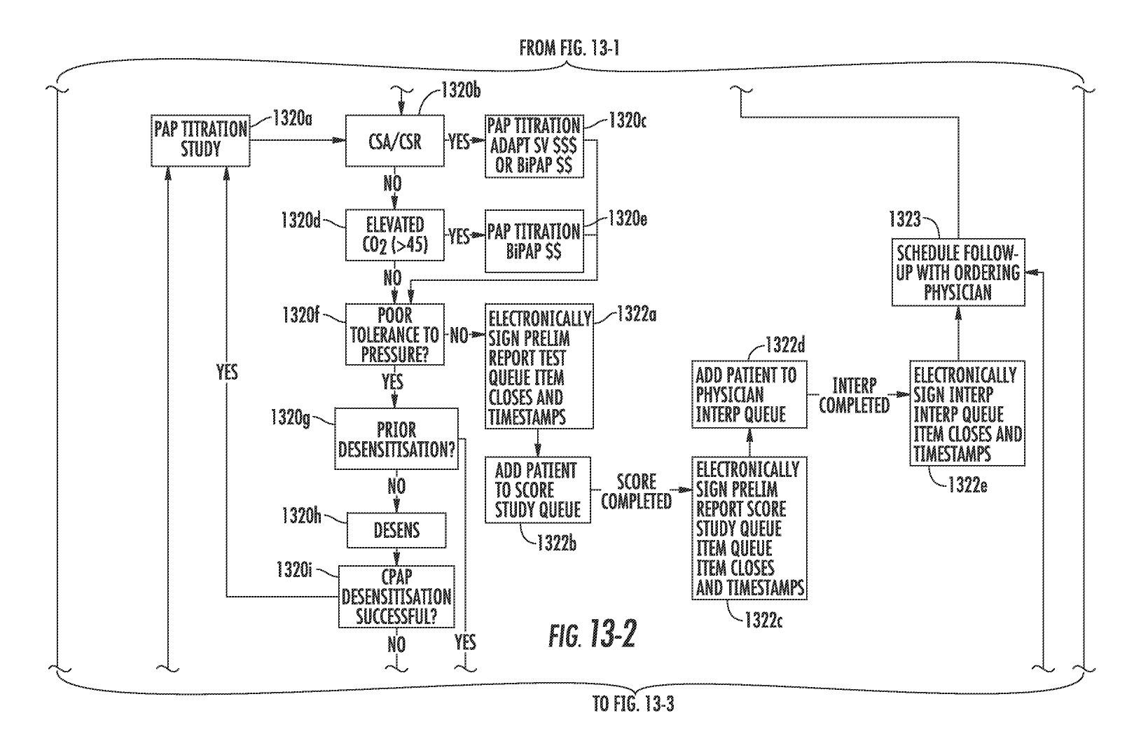

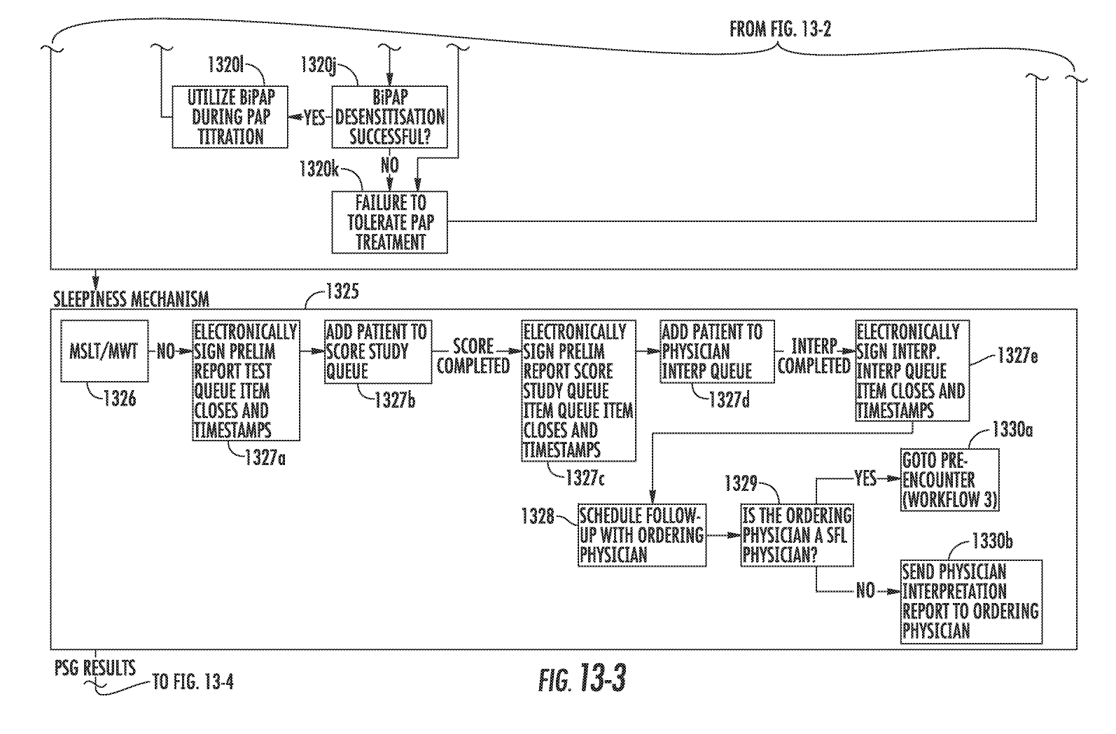

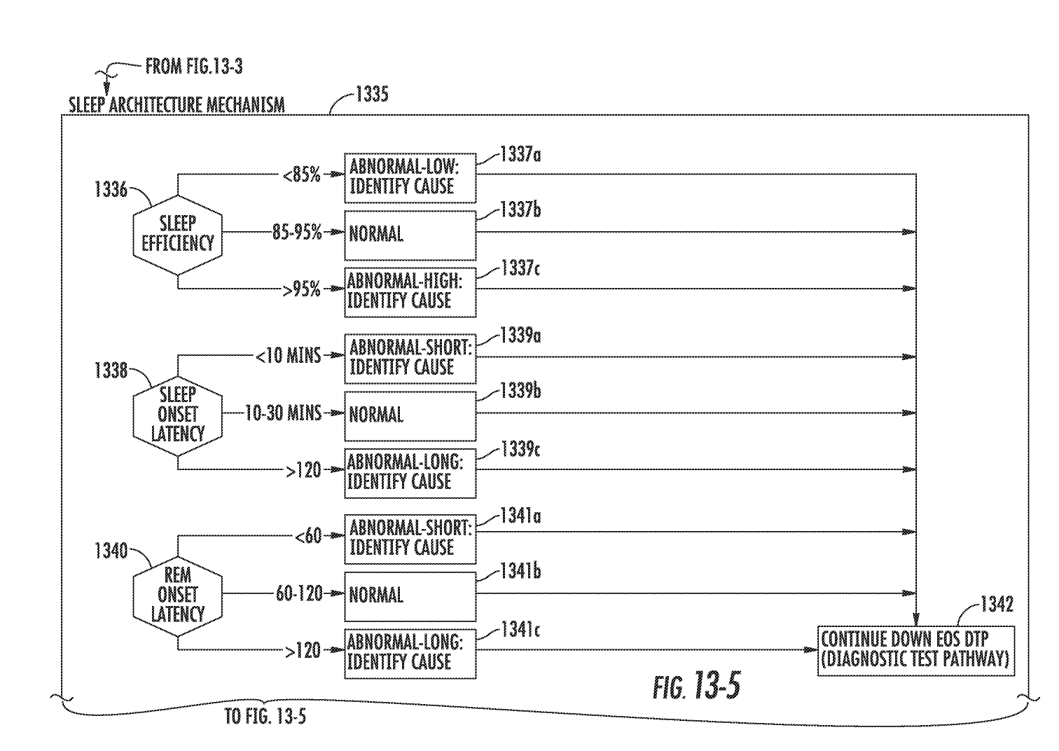

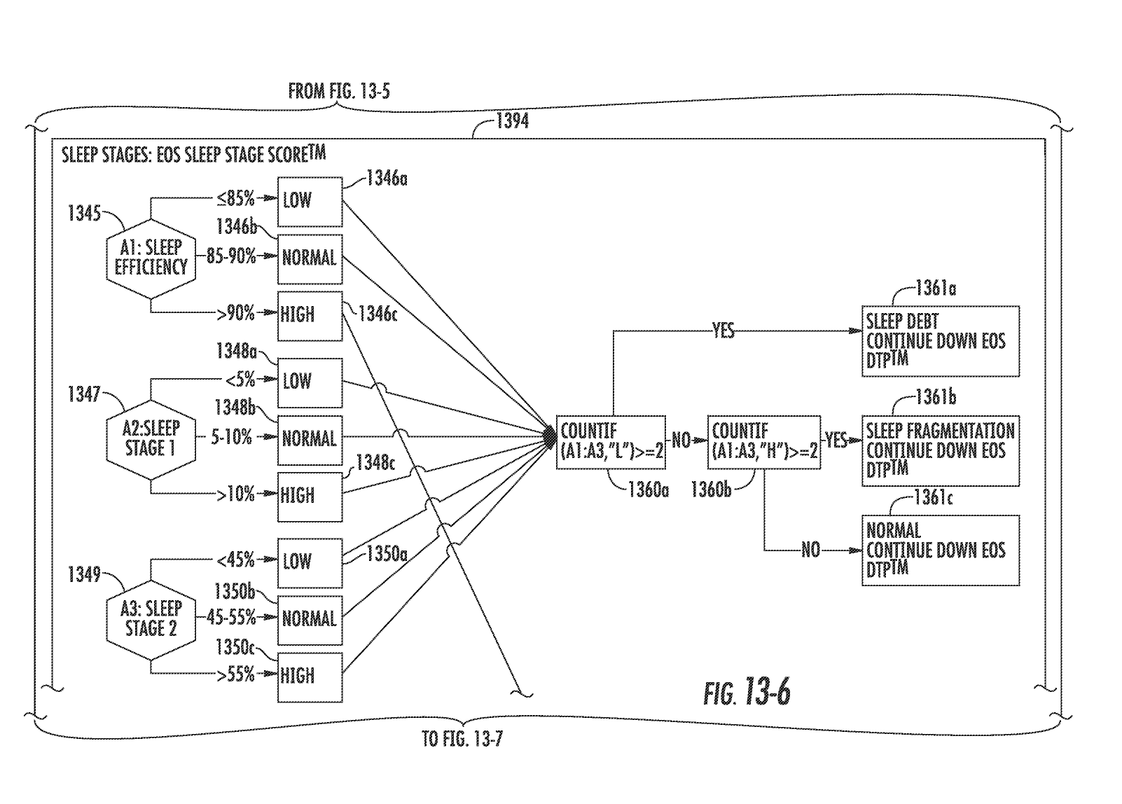

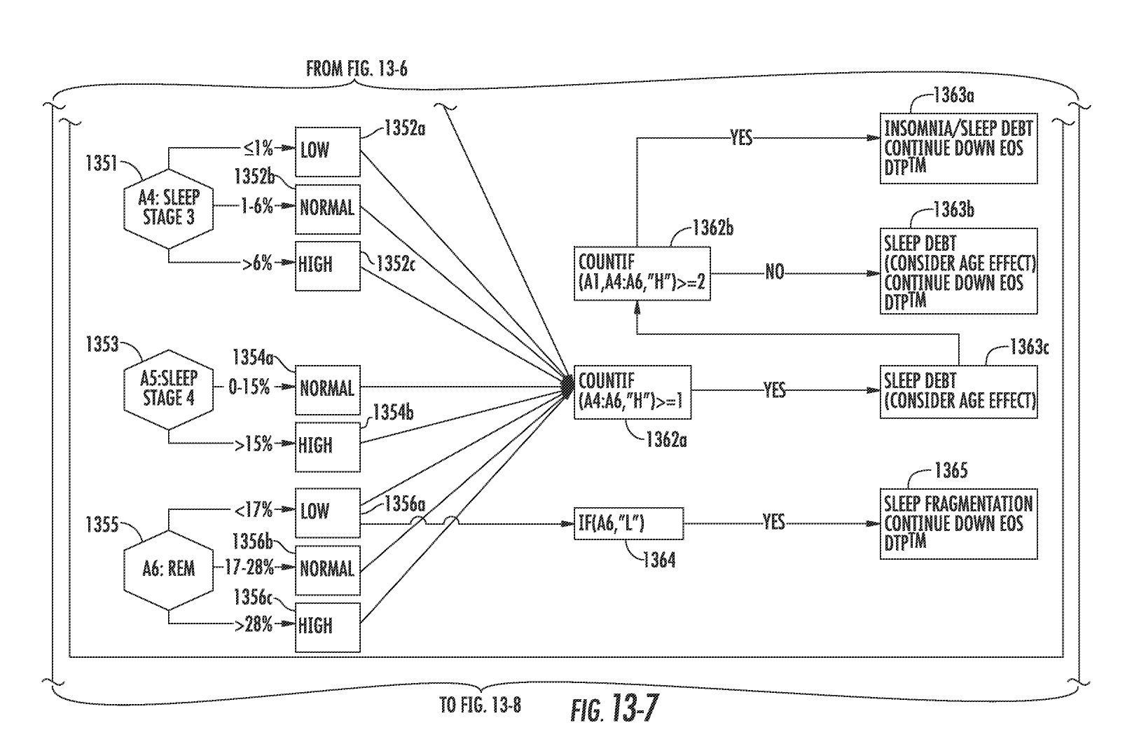

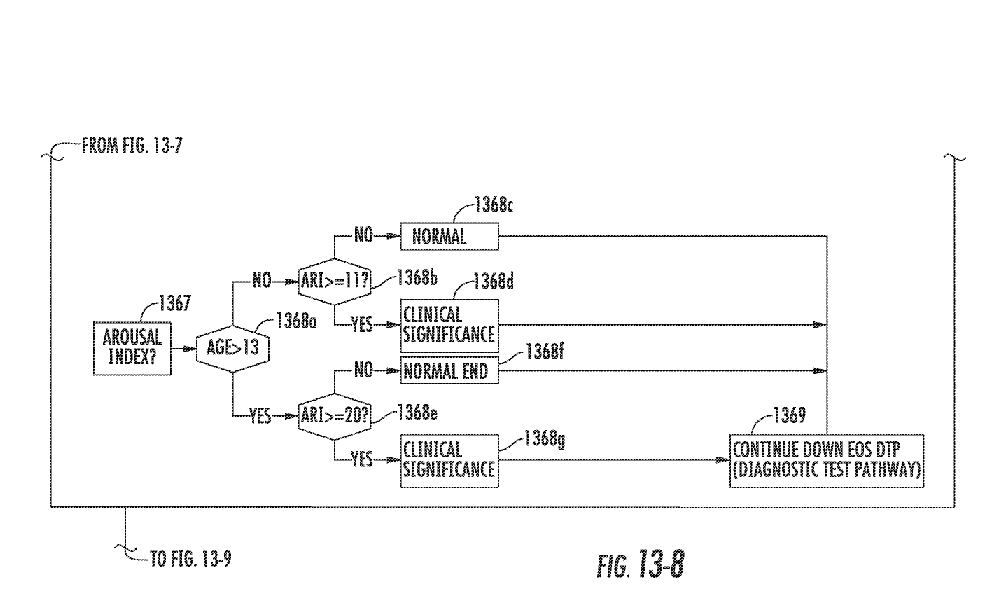

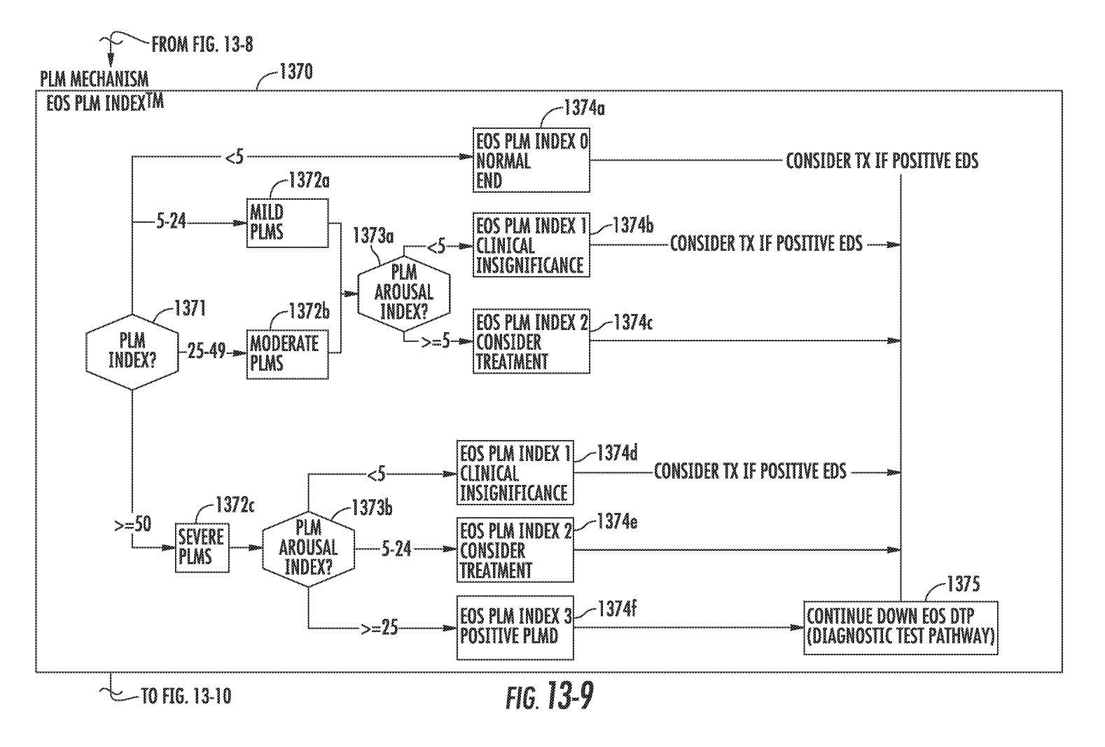

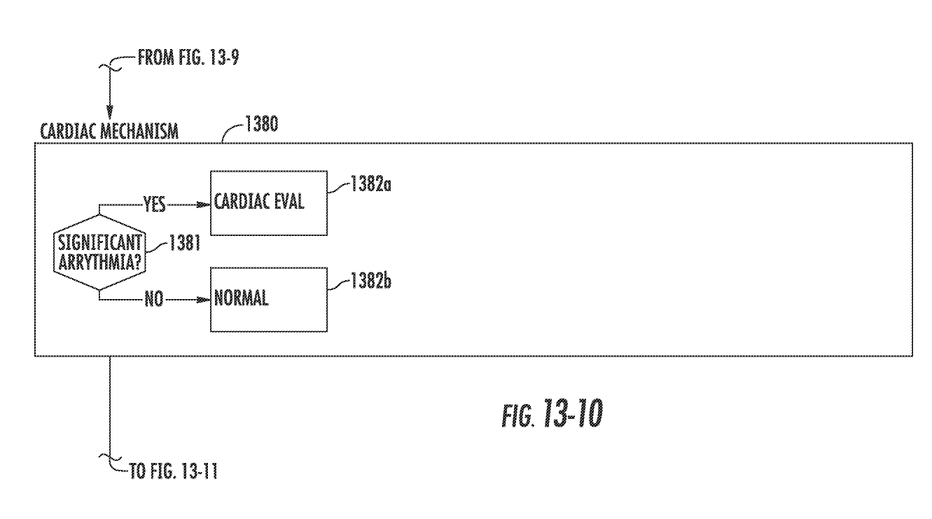

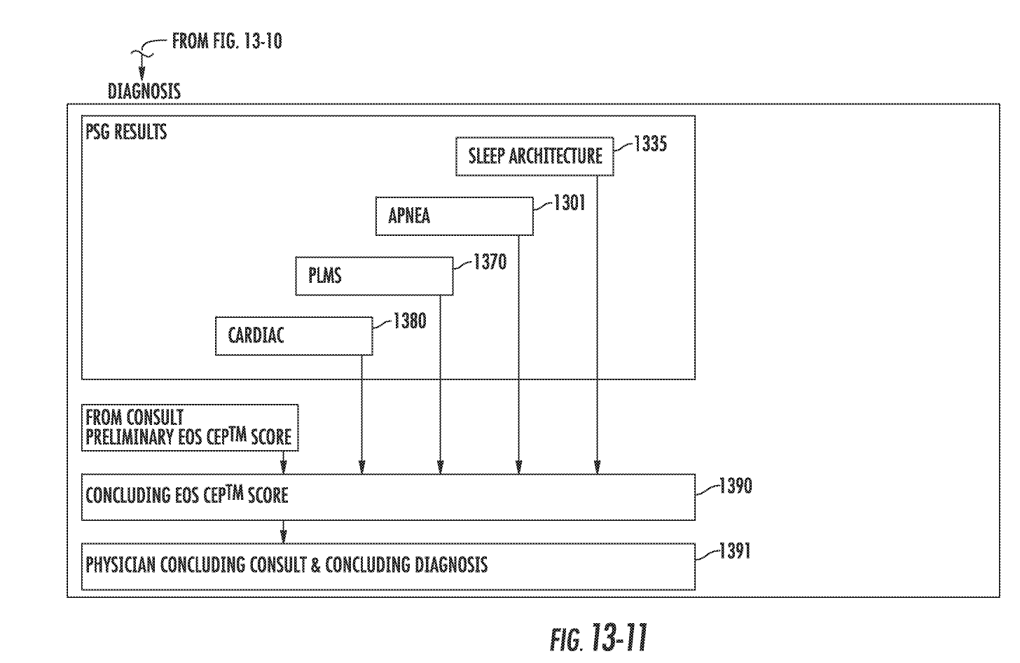

FIG. 13 illustrates diagnostic testing pathway (DTP) 1300 from workflow 13 (WF13) which provides a method for the interpretation and management of diagnostic data acquired via polysomnographic recording (PSG), positive airway pressure (PAP) titration, periodic limb movement, cardiac and sleep architecture.

Apnea mechanism module 1301 utilizes oxygen saturation and the patient's apnea hypopnea index (AHI) to define the significance of the patient's sleep related breathing disorder. Using the AHI value the method incorporates a sleepiness scale and patient social and past medical history and physical exam findings to suggest treatment pathways. In block 1302, a polysomnogram (PSG) is administered. Preferably, the PSG is a nocturnal polysomnogram (NSPG). The oxygen saturation is measured in block 1303 and a titrate oxygen is administered in block 1304 if the oxygen saturation falls below a threshold level. When the study is in progress from block 1305, intra acquisition of the patient's hypopnea index (AHI) is performed using blocks 1306a and 1306b. When the study is finished blocks 1307a-1307c are performed to determine a preliminary test report, add a patient score and a physician interpretation to the report and electronically sign the preliminary report, patient score and physician interpretation.

In block 1308, the AHI score is interpreted. In blocks 1309a-1309d, a follow-up with an ordering physician is determined. In blocks 1311 and 1312a, if the ordering physician participates in system 100, the method proceeds to the pre-encounter module 300. Alternatively, in block 1312b a non-participating physician is forwarded the test report.

Apnea mechanism module 1301 also incorporates a method to suggest the proper positive airway pressure (PAP) device to be utilized for treatment titration studies. In blocks 1320a-1320d, a test report, score and physician interpretation is generated and a signature is applied to the report and score and physician interpretation, in blocks 1322a-1322e. In block 1323a, a follow-up with an ordering physician id determined.

Sleepiness mechanism module 1325 describes the process associated with multiple sleep latency testing and maintenance of wakefulness testing. In block 1326, multiple sleep latency testing (MSLT) and maintenance of wakefulness testing (MWT) is administered. Blocks 1327a-1327e are performed to determine a preliminary test report, add a patient score and a physician interpretation to the report and electronically sign the preliminary report. In block 1328 a follow-up with an ordering physician is determined. In block 1329 and 1330a if the ordering physician is participating in system 100, the method proceeds to the pre-encounter module 300.

Sleep architecture mechanism module 1335 evaluates sleep efficiency, sleep onset latency, and REM onset latency, evaluates the sleep stages utilizing a sleep stage score and evaluates arousal index, thereby identifying the need for further evaluation depending on clinical significance. In blocks 1336 and 1337a-1337c, sleep efficiency is classified. In blocks 1338 and 1339a-1339c, sleep onset latency is classified. In blocks 1340 and 1341a-1341c, REM onset latency is classified. In block 1342, the information for sleep architecture mechanism module 1335 leads the clinician to continue diagnostic test pathway 1300 to determine sleep stage scores in sleep stage on score module 1344.

In blocks 1345 and 1346a-1346c, sleep efficiency is classified. For example, sleep efficiency can be classified as low, normal or high. In blocks 1347 and 1348a-1348c, a sleep stage 1 score is classified. In blocks 1349 and 1350a-1350c, a sleep stage 2 is classified. In blocks 1351 and 1352a-1352c, a sleep stage 3 is classified. In blocks 1353 and 1354a and 1354b, a sleep stage 4 is classified. In blocks 1357 and 1358a-1358c, a REM value is classified. Sleep efficiency values of low and normal are combined with sleep stage 1 and sleep stage 2 scores in blocks 1360a and 1360b to determine sleep conditions 1361a-1361c. A sleep efficiency value of high scores are combined with sleep stage 3, sleep stage 4 scores and REM values in blocks 1362a and 1362b to determine sleep conditions 1363a-1363c of insomnia or sleep debt.

A low REM value is interpreted in block 1364 for determining sleep conditions 1365. An arousal index (ARI) is performed in block 1367. The ARI is classified in blocks 1368a based on age and value. In block 1369, the information from the ARI leads the clinician to continue on diagnostic test pathway 1300 to periodic limb movement mechanism module 1370.

Periodic limb movement (PLM) mechanism module 1370 incorporates a periodic limb movement (PLM) index to evaluate the number of PLMs and the associated arousals to suggest clinical significance and/or treatment consideration. In block 1371a periodic limb movement (PLM) index is determined. Blocks 1372a-1372c are performed to classify the PLM index. In blocks 1373a and 1373b a PLM arousal index is determined. The classification of the PLM index and/or the PLM arousal are used to determine clinical significance and treatment considerations in blocks 1374a-1374f. In block 1375, the information from the PLM and PLM arousal index leads the clinician to continue on diagnostic test pathway 1300 to cardiac mechanism module 1380.

The cardiac mechanism module 1380 identifies the presence of arrhythmia which, if present, suggests the need for further cardiac evaluation. Cardiac arrhythmia is determined in block 1381 and classified in blocks 1382a and 1382b.

The outcome of DTP 1300 of sleep architecture module 1335, apnea module 1301, periodic limb movement module 1370 and cardiac module 1380, combined with the preliminary clinical evaluation pathway (CEP) score, which takes the polysomnographic results into account, provides a concluding clinical evaluation pathway (CEP) 1390 score. Clinical evaluation pathway (CEP) 1390 score can be used by a physician for diagnosis and treatment option in block 1391.

The patient returns to an EOS facility to review polysomnographic data results with clinical staff. The clinical staff then considers all available data and creates a treatment plan.

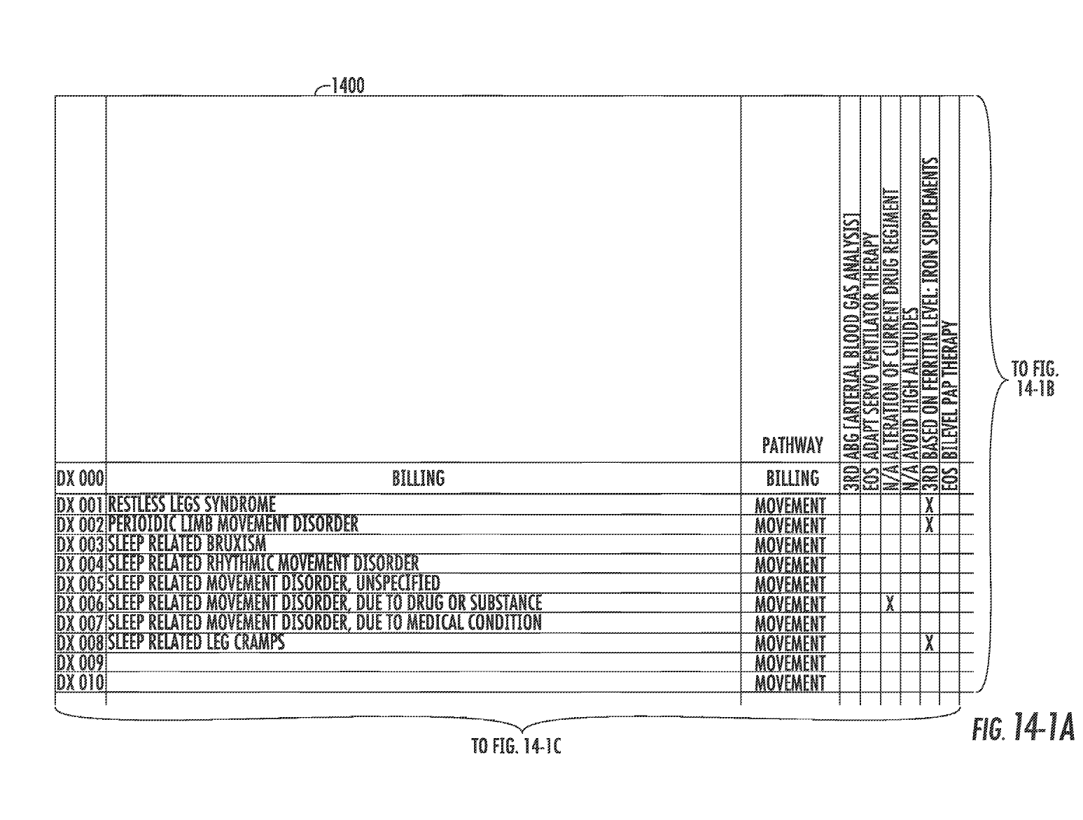

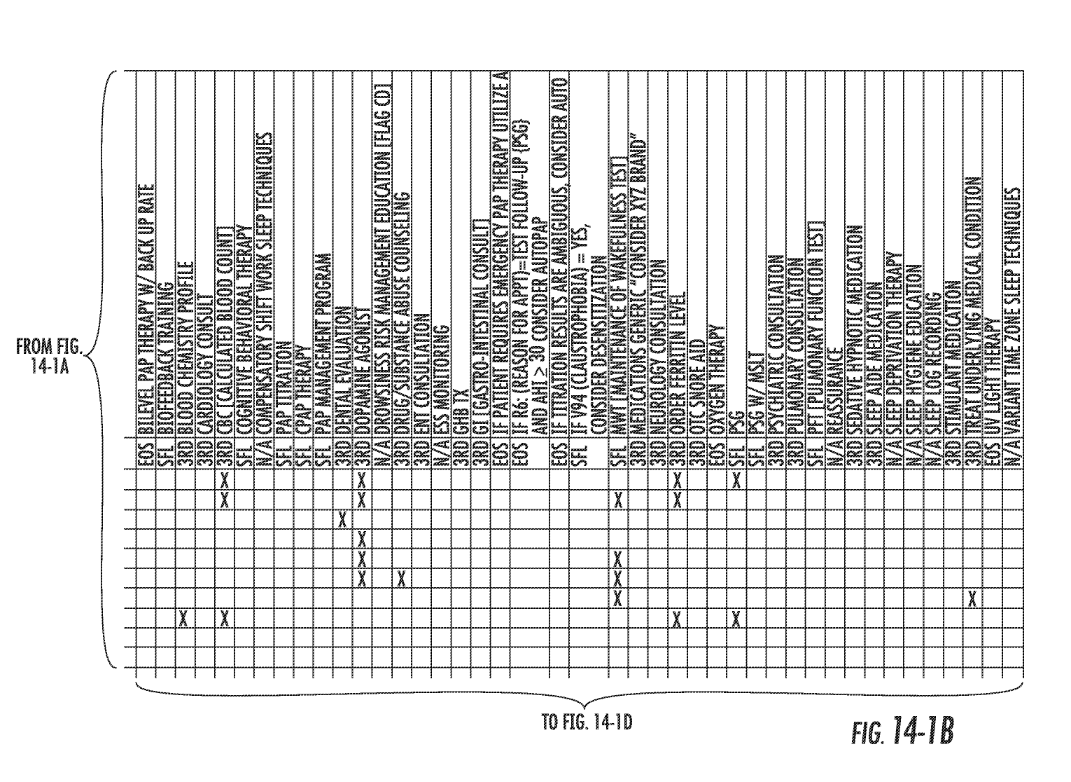

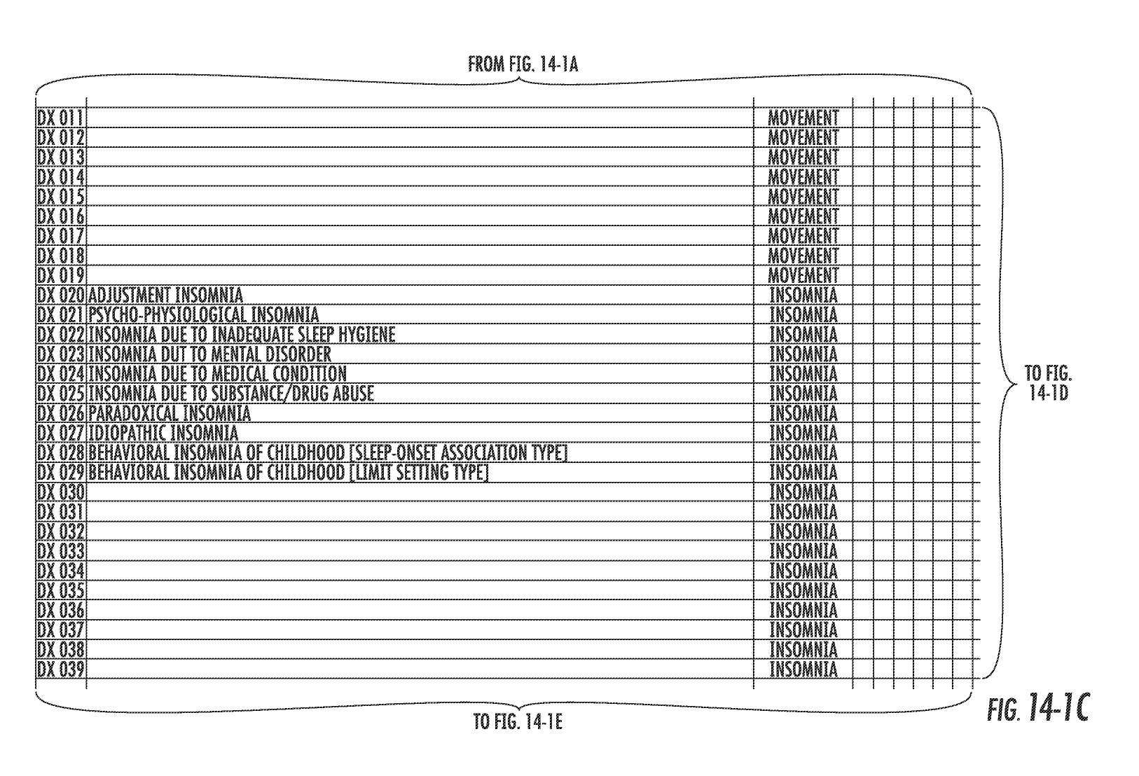

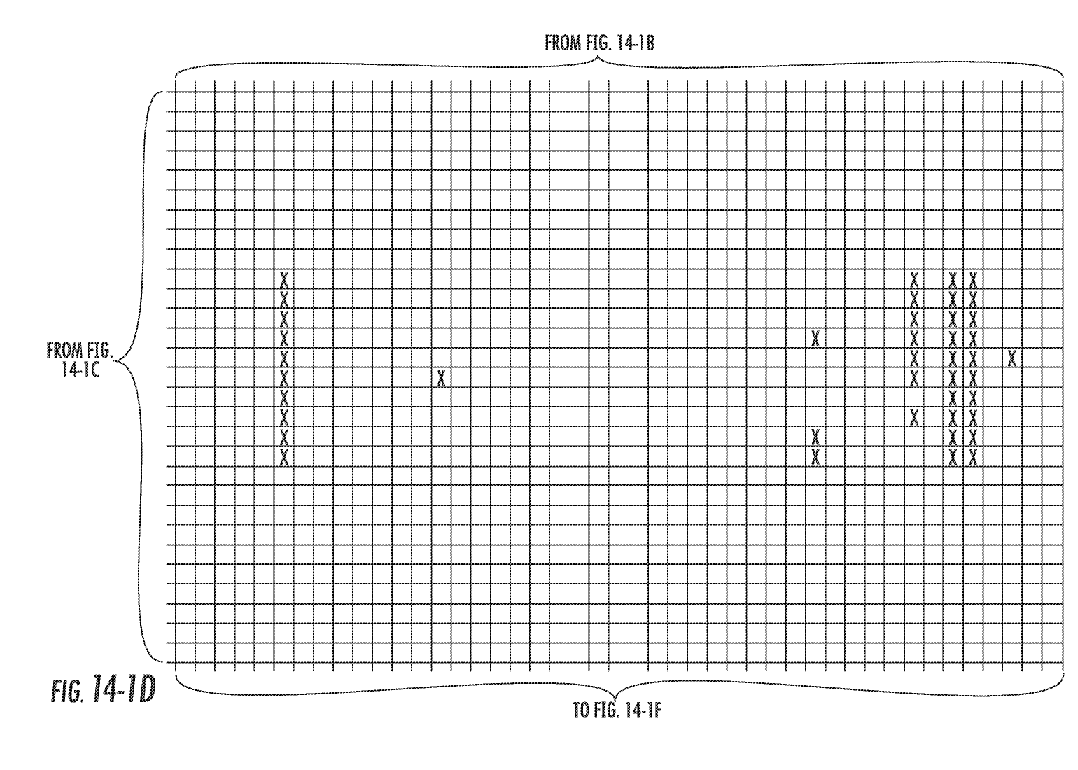

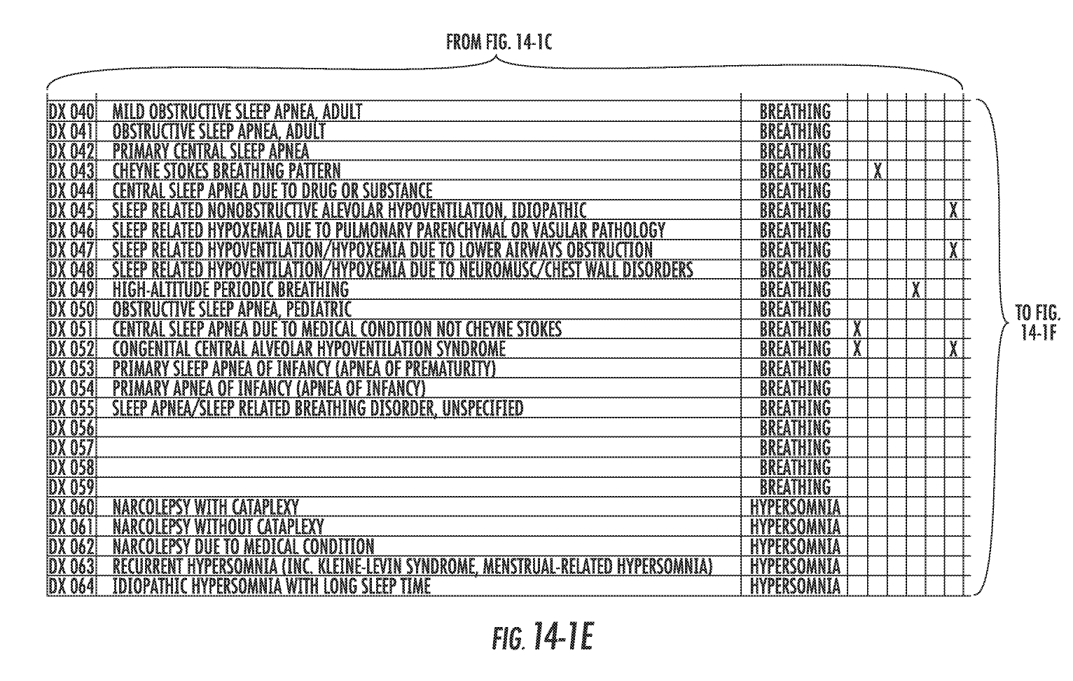

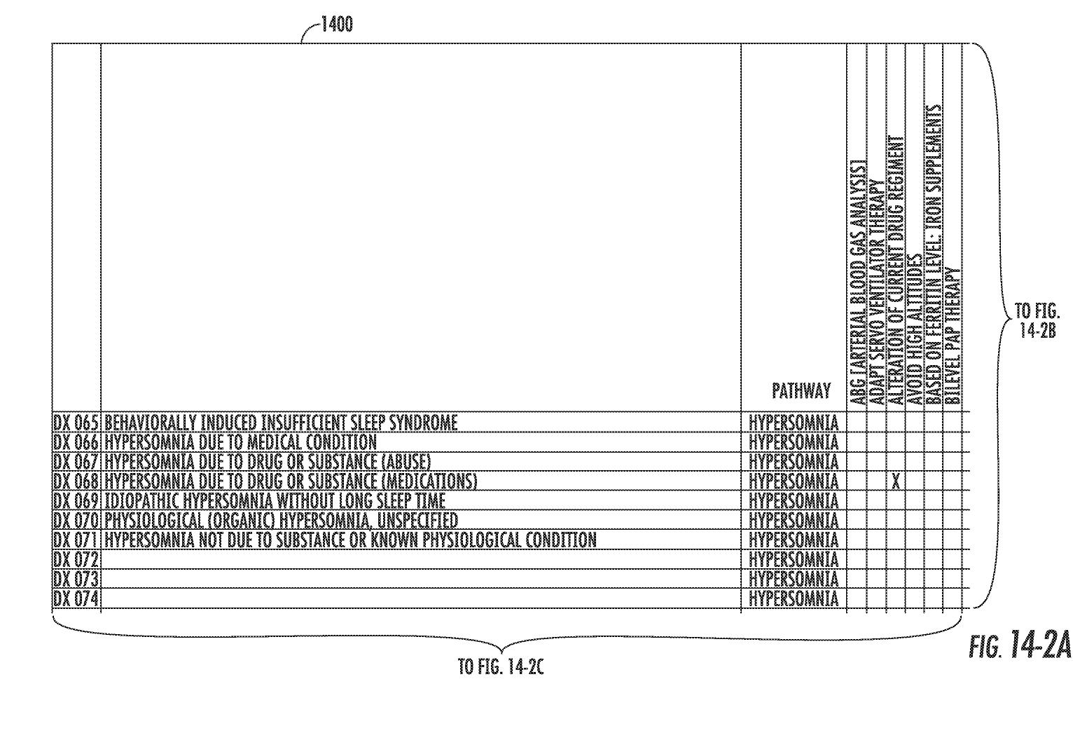

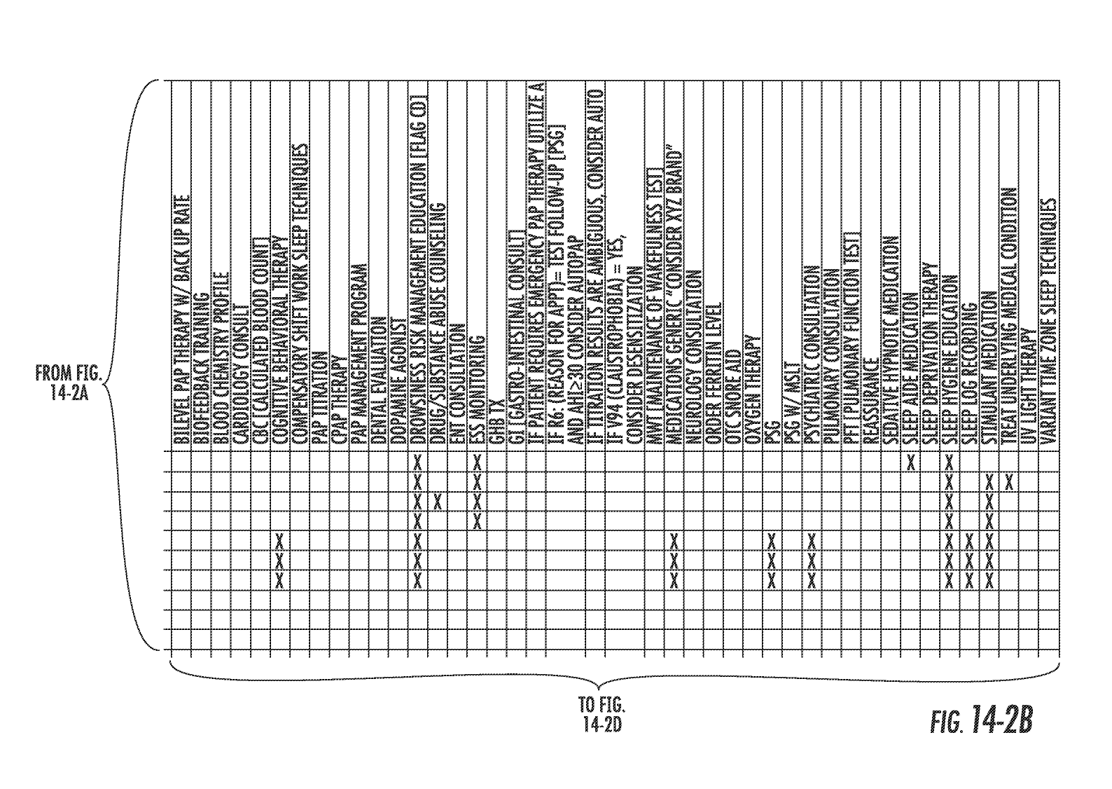

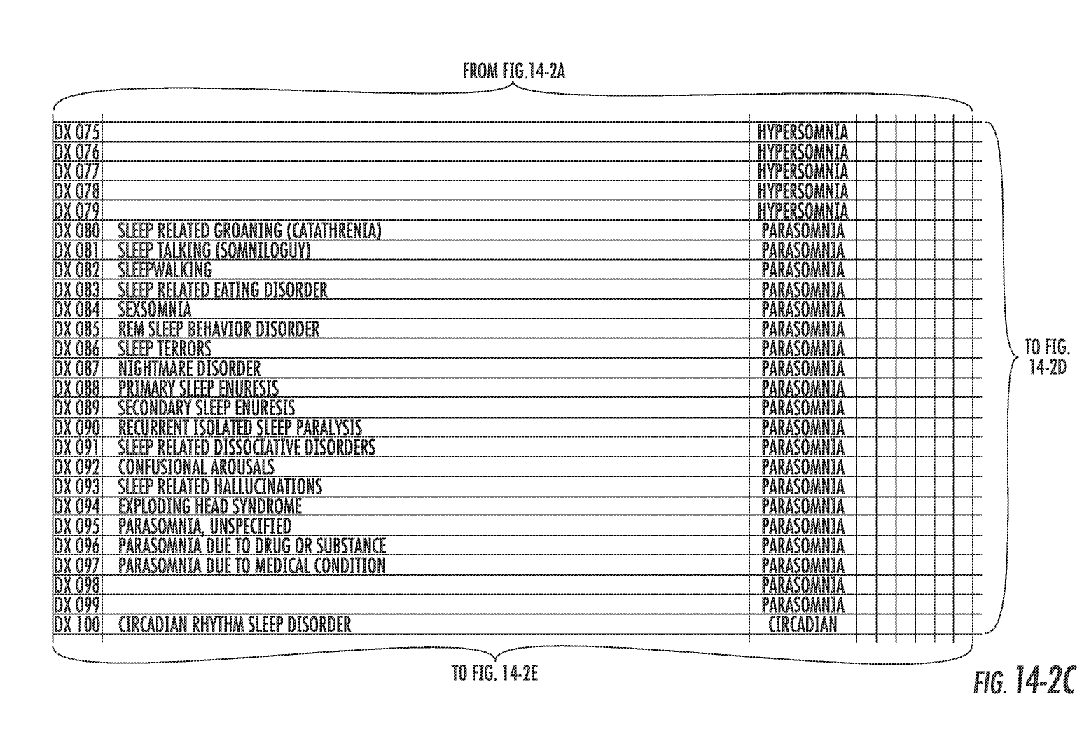

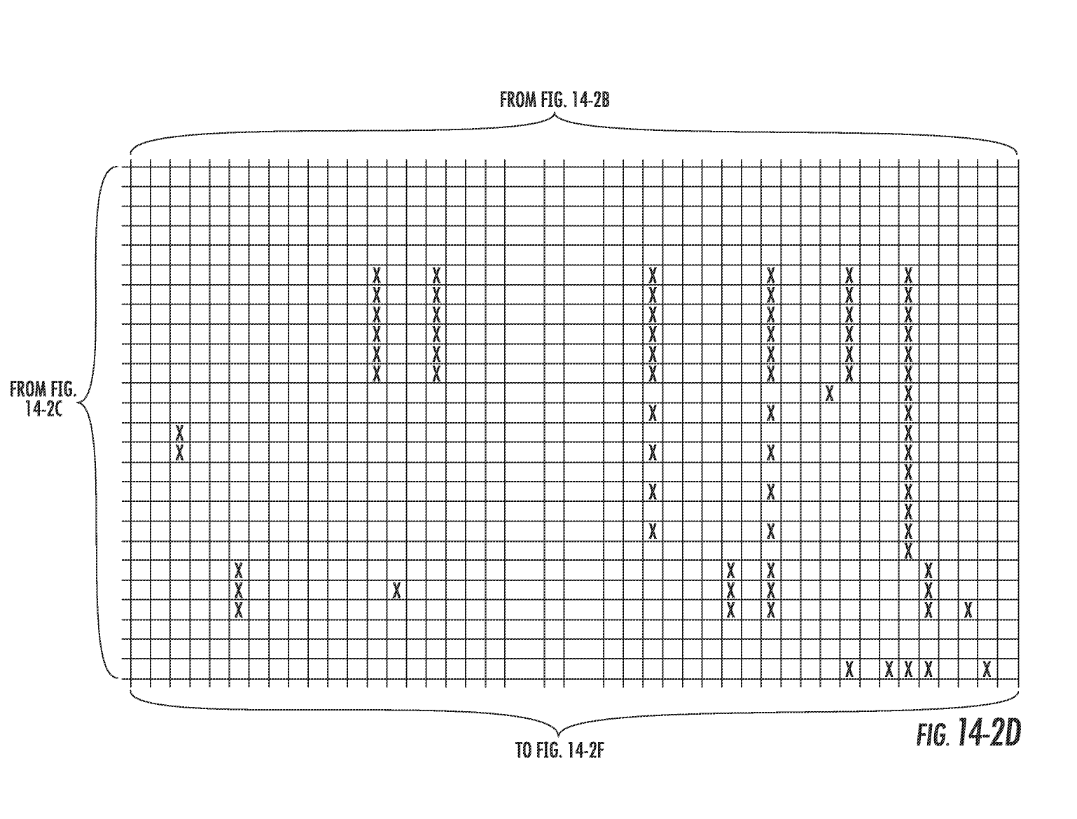

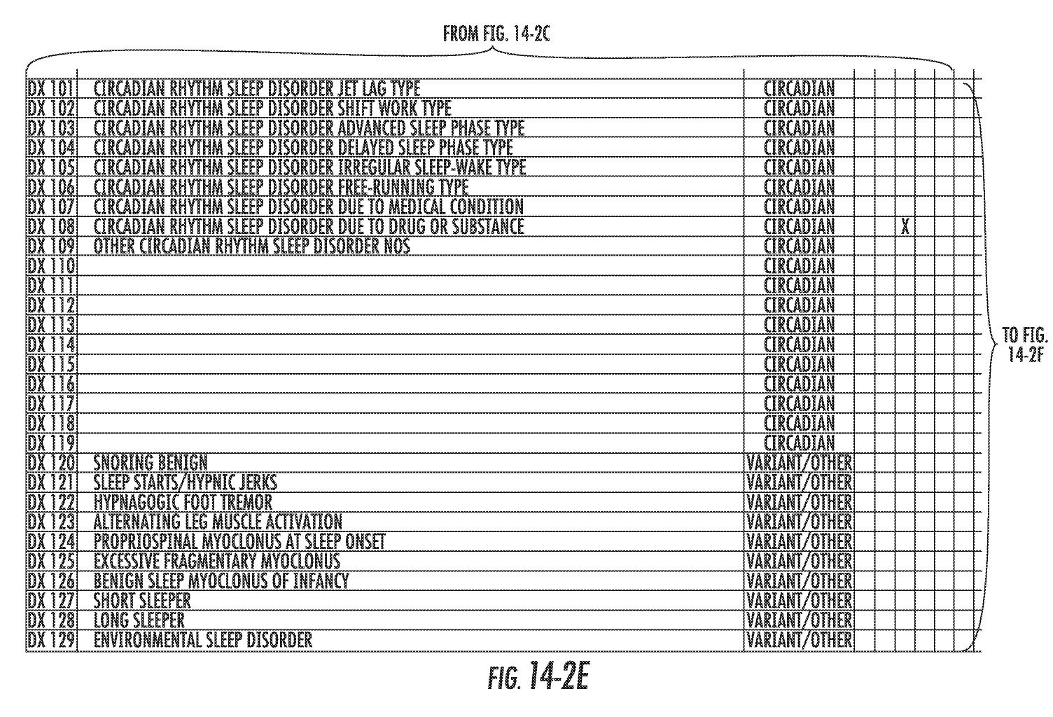

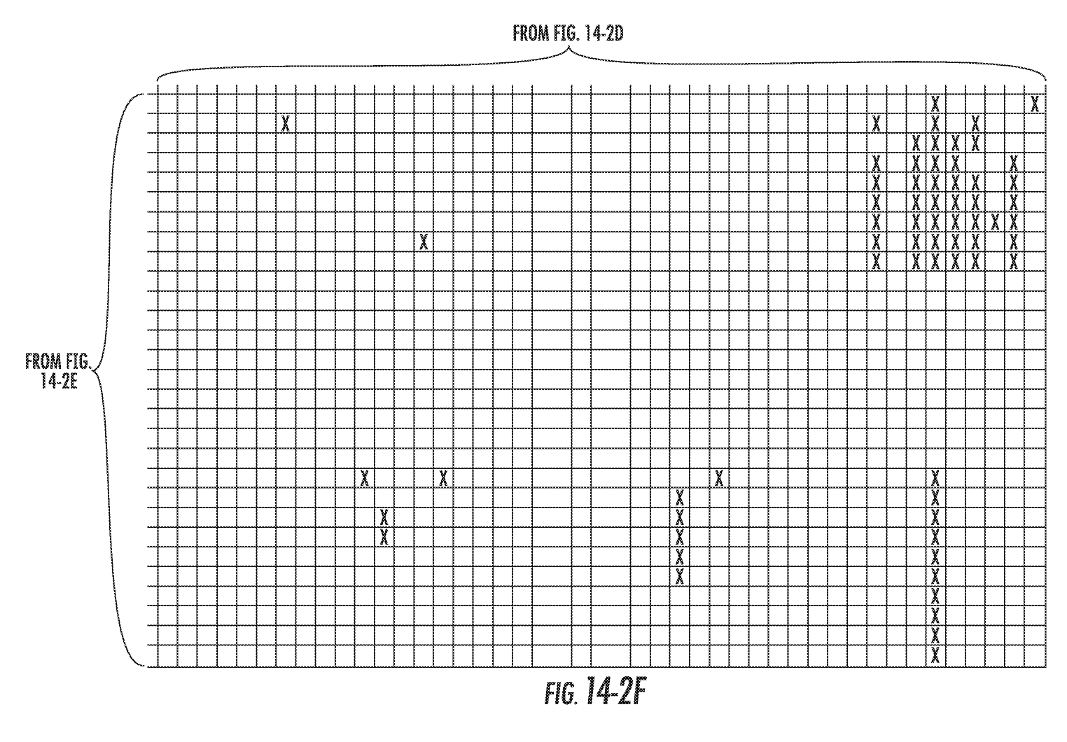

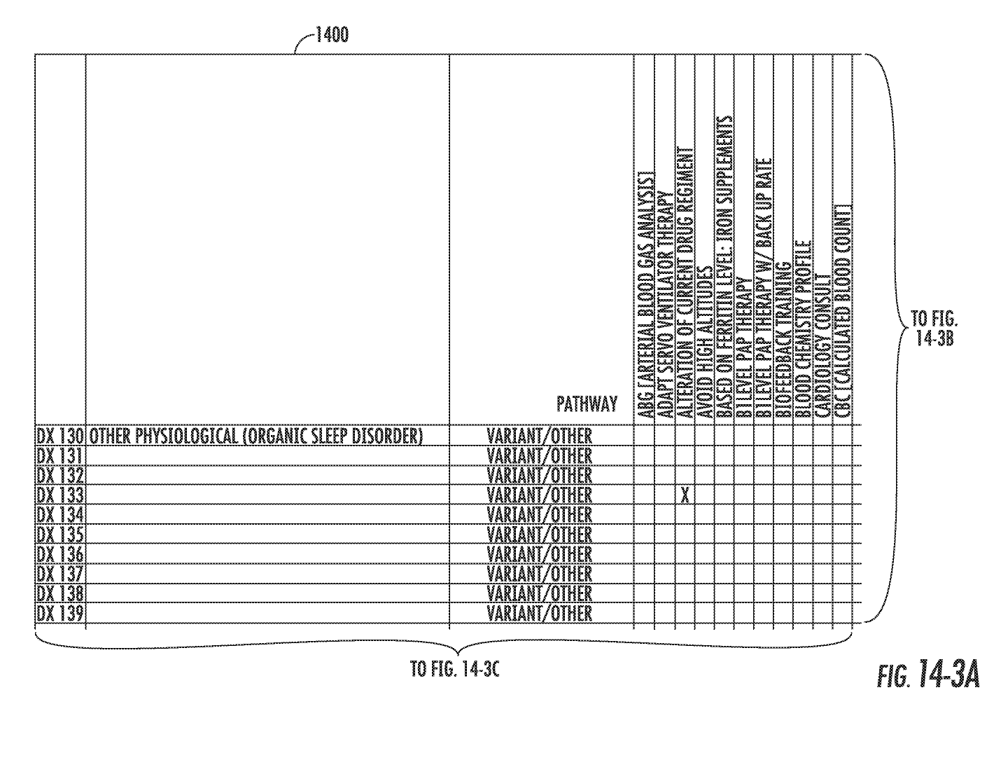

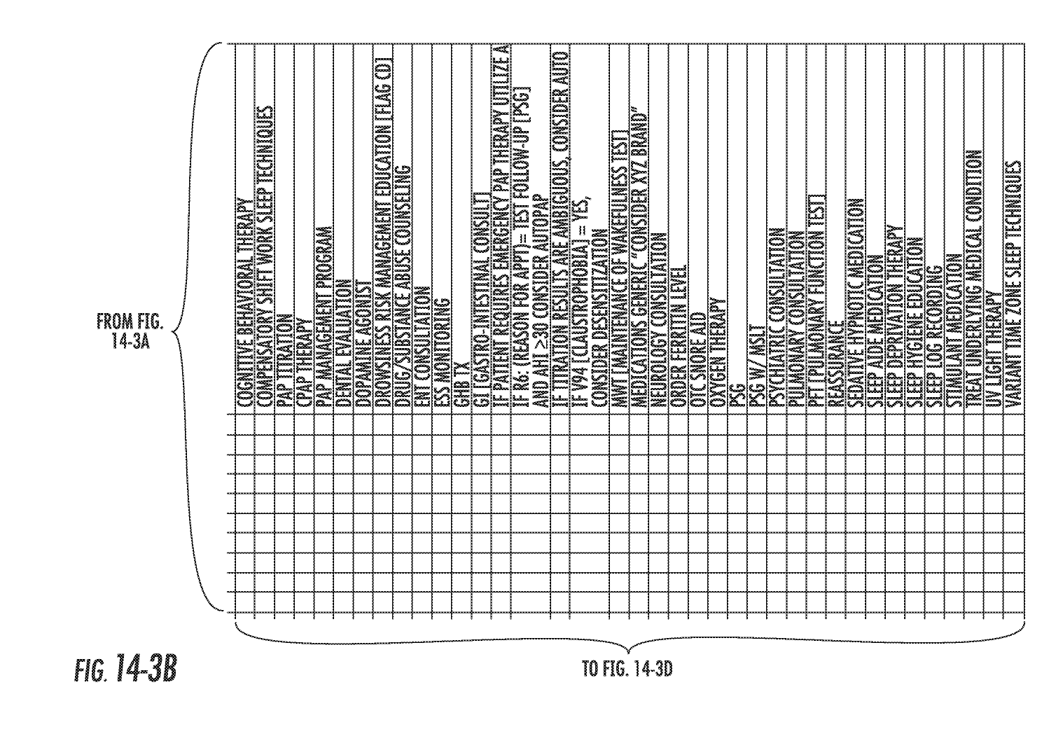

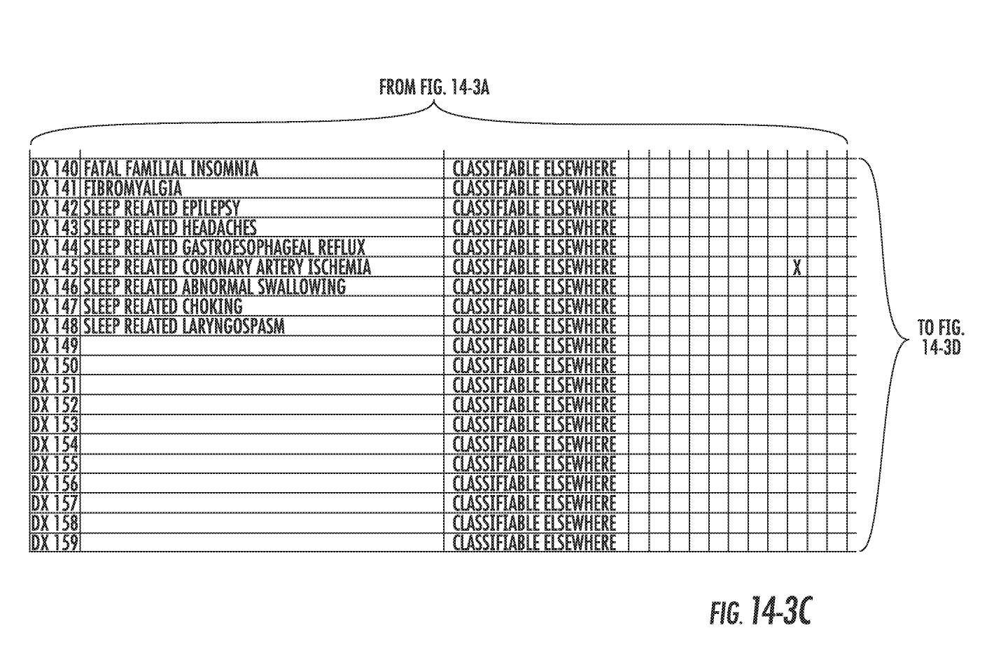

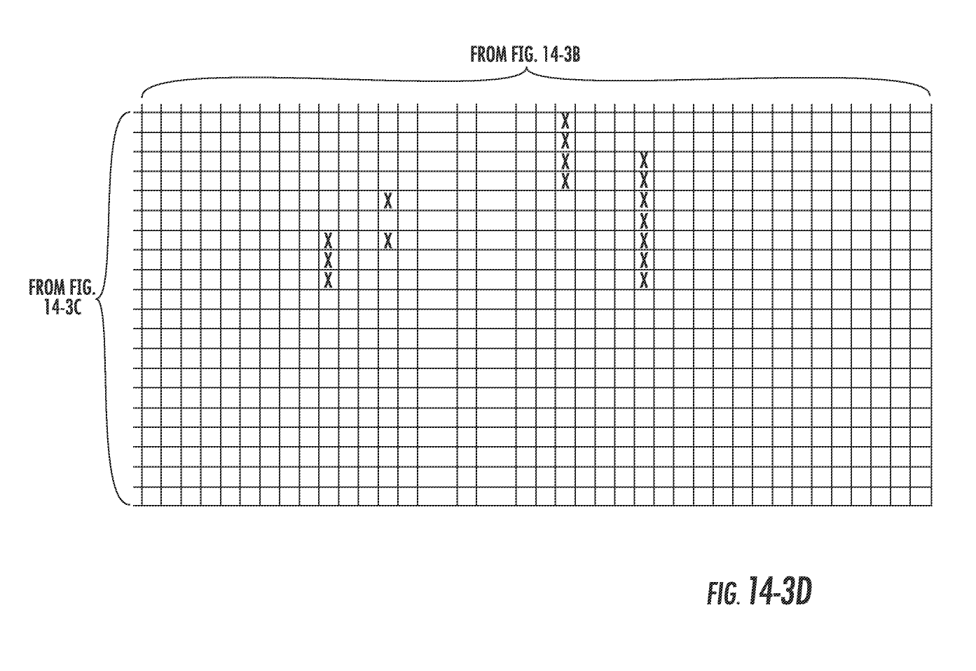

FIG. 14 illustrates a schematic graph of diagnosis-to-treatment mapping module 1400 shown in workflow 14 (WF14) to provide suggested treatment modalities for each disease classification identified within clinical evaluation pathways. Workflow 14 permits the association of specific brand names of outside entities with treatment options such as prescription medication, positive airway pressure equipment and/or devices used to treat or manage sleep disorders.

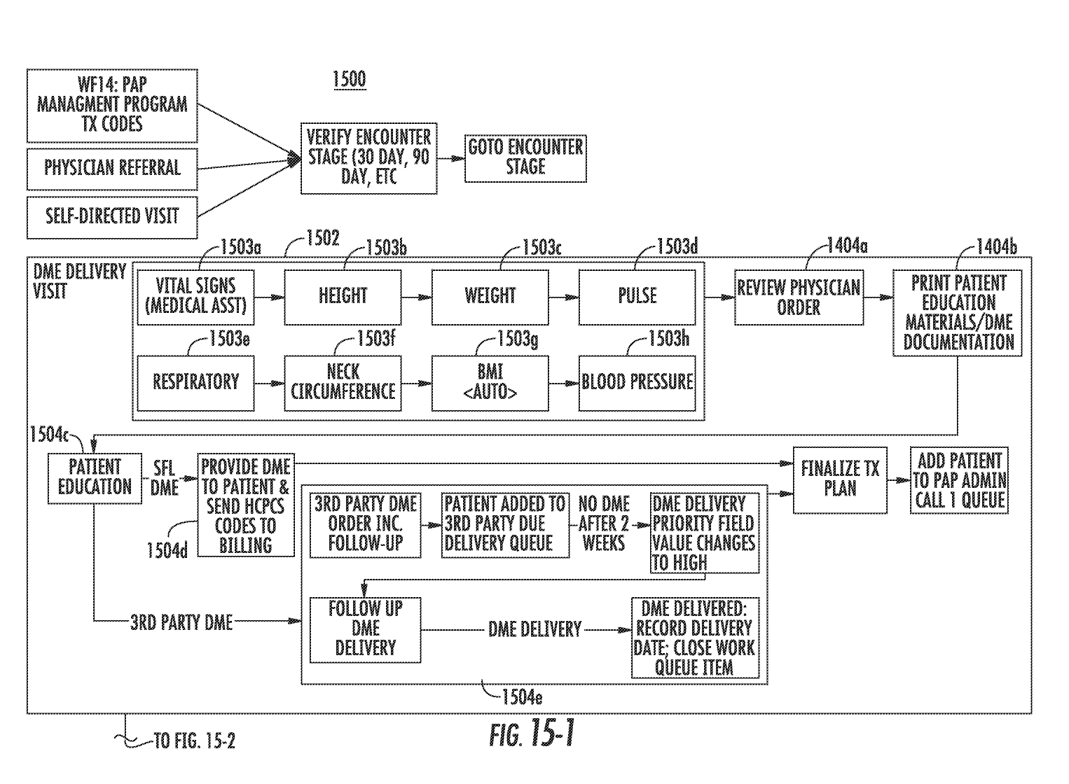

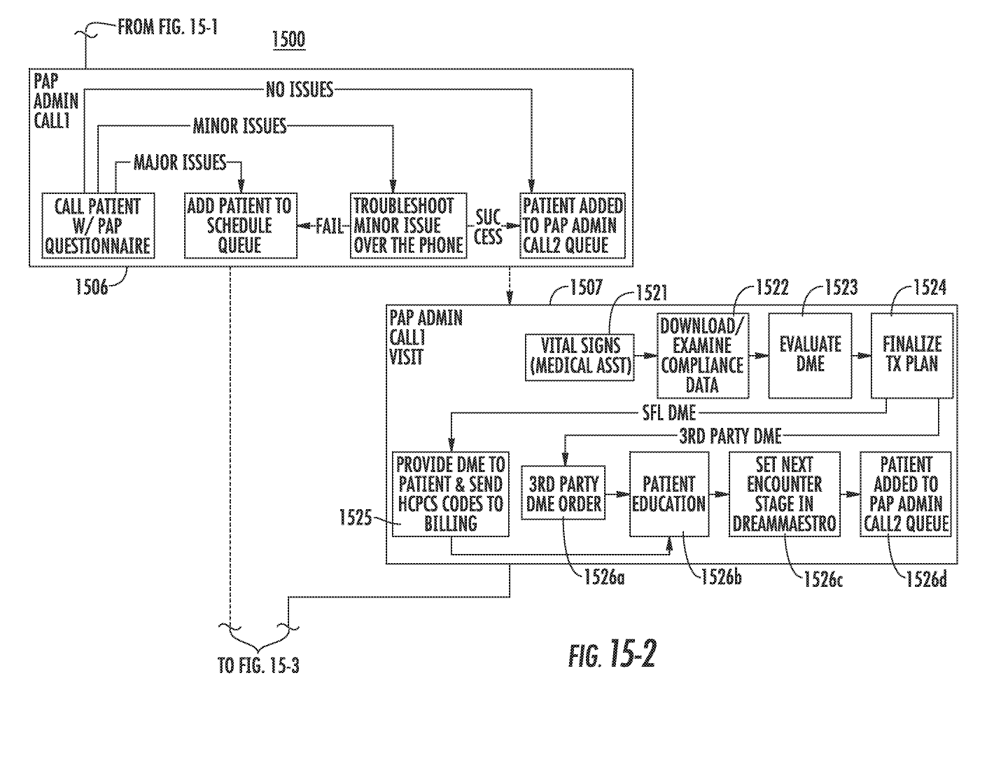

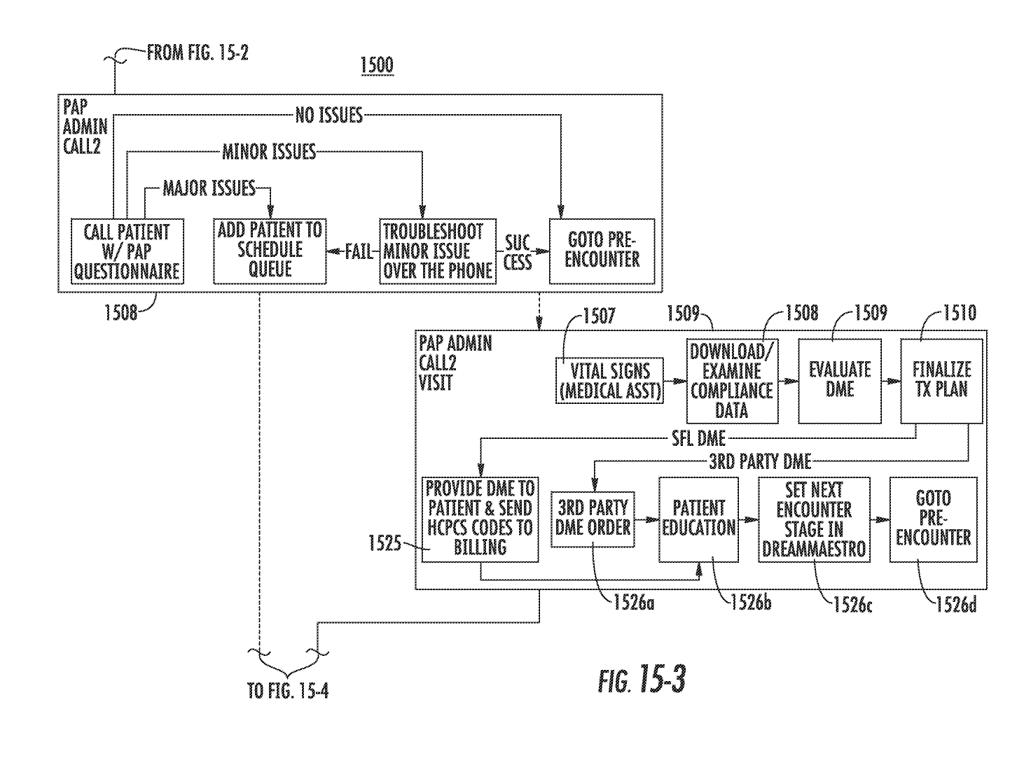

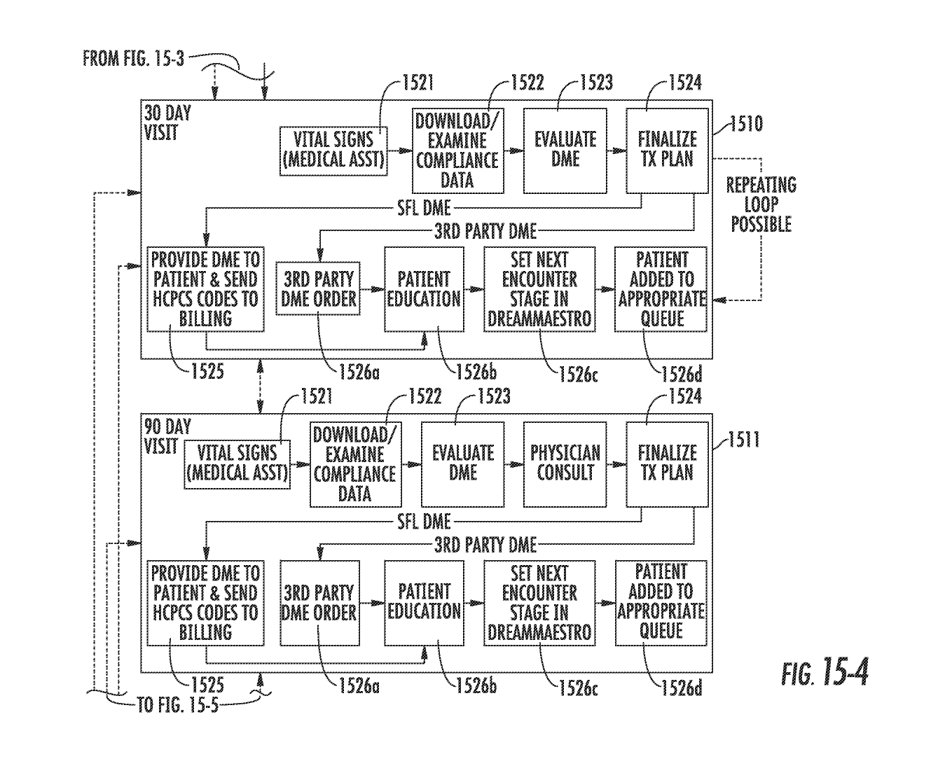

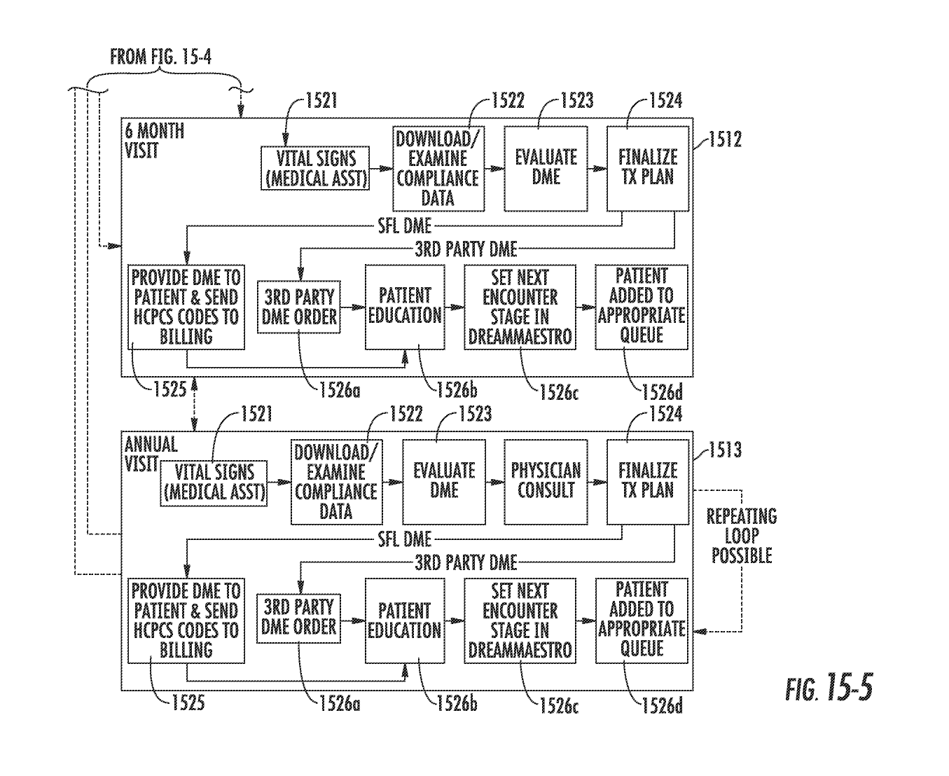

FIG. 15 is a schematic diagram of PAP administration module 1500 described in workflow 15 (WF15) which provides a method for patient evaluation, equipment delivery and electronic tracking. The method considers patient success with positive airway pressure (PAP) treatment. This defines the timeframes in which a patient is contacted by the PAP administration staff either via telephone interview or scheduling for a face-to-face encounter.

PAP administration module 1500 tracks patient compliance with PAP treatment either via wireless tracking systems, data card downloads, direct machine analysis and completion of a PAP follow-up template. The patient can be monitored for replacement equipment and treatment alterations.

DME delivery module 1502 provides delivery of medical equipment. Vital signs are received in blocks 1503a-1503h. Review of the delivery order distribution of the DME or third party distribution of the DME are performed in blocks 1504a-1504e. In block 1505, a plan is finalized and a positive airway pressure (PAP) first call is initiated in module 1506. A PAP administration visit from call 1 is scheduled in module 1507. A respective follow-up PAP second call is initiated in module 1508 and a PAP administration visit from call 2 is scheduled in module 1509.

Thereafter, a 30-day visit is initiated in module 1510, a 90-day visit is initiated in module 1511, a six-month visit is initiated in module 1512 and an annual visit is initiated in module 1513. Each of the scheduled visits include block 1520 for determining vital signs, block 1521 for downloading and examining compliance data, block 1522 to evaluate distribution of medical equipment, block 1423 for physician consultation, block 1524 to finalize a treatment plan, block 1525 to provide DME to patient and send HCPCS codes to billing and blocks 1526a-1526d are determined for third-party ordering and education related to DME.

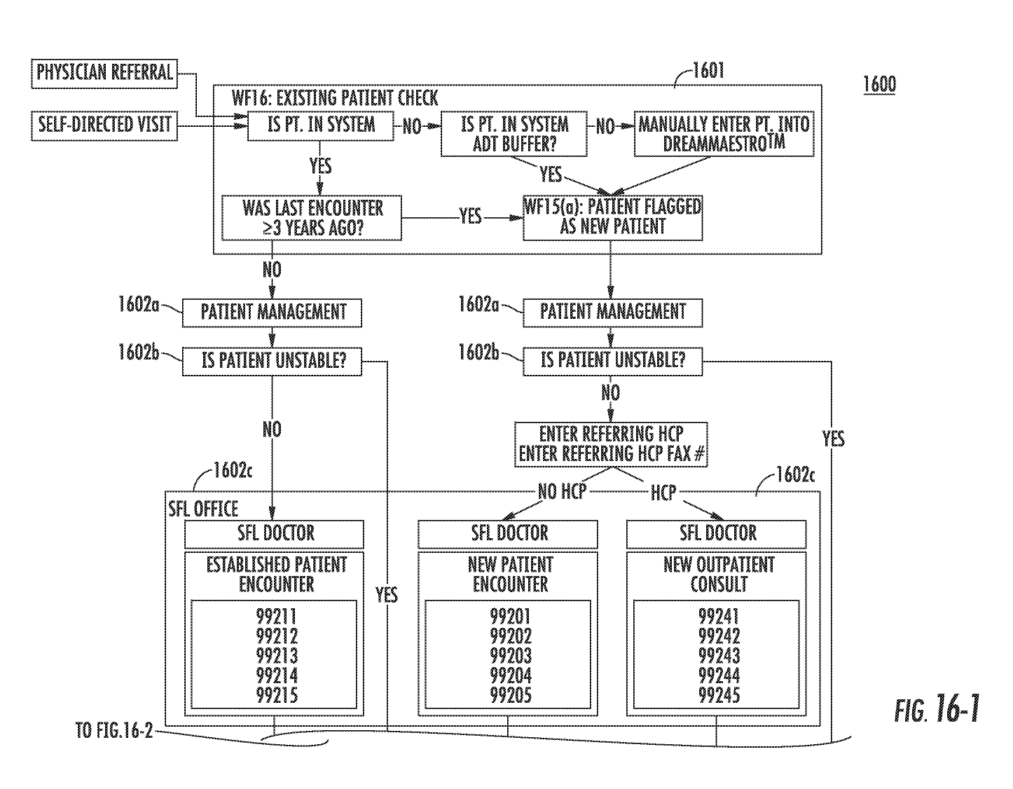

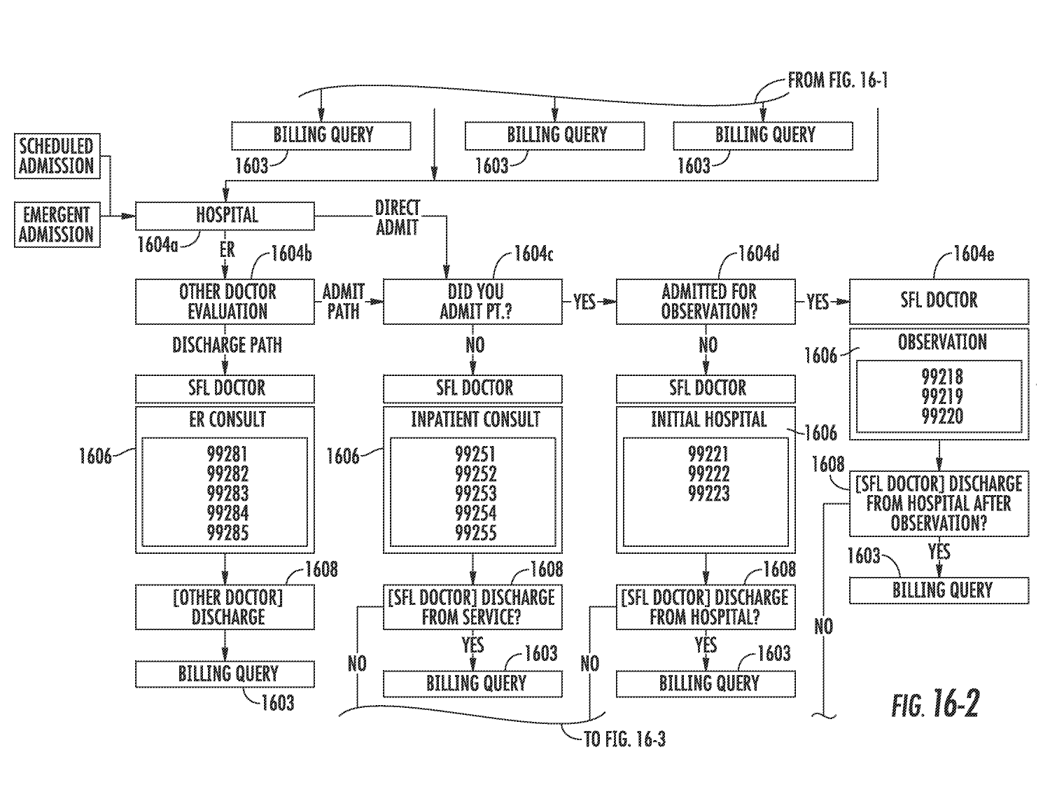

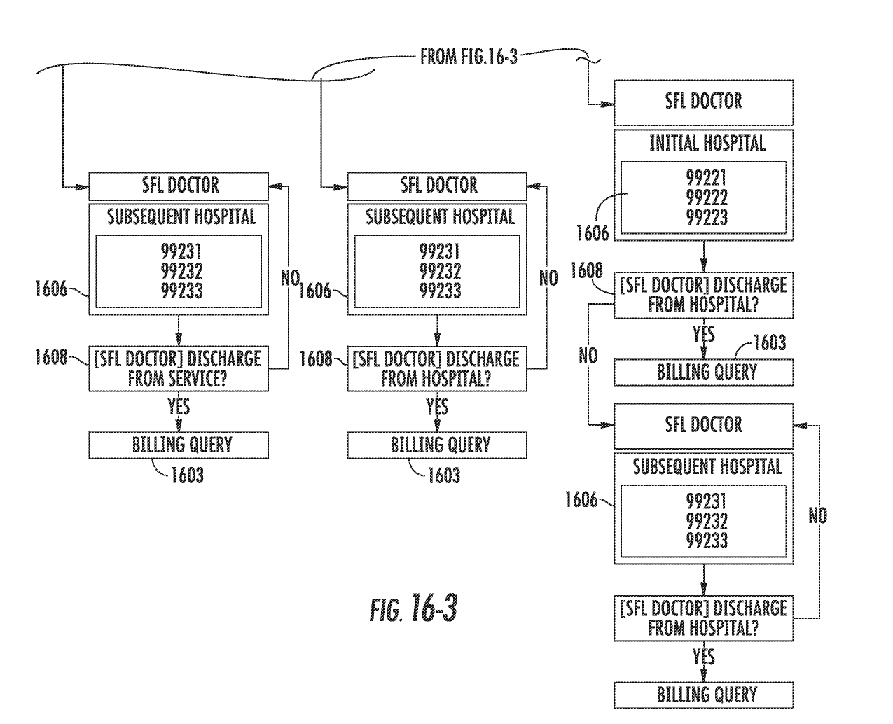

FIG. 16 illustrates a schematic diagram of an encounter classification module 1600 from workflow 16 (WF16). In module 1601, an existing patient check is performed. In blocks 1602a-1602d a doctor is established and the encounter type is established. In block 1603, a billing query is determined. Blocks 1604a-1604e determine admission to a hospital. Block 1606 determines a doctor and establishes an encounter type. Doctor discharge is performed in block 1608. Blocks 1610a-1610e determine the billing codes used in the billing query.

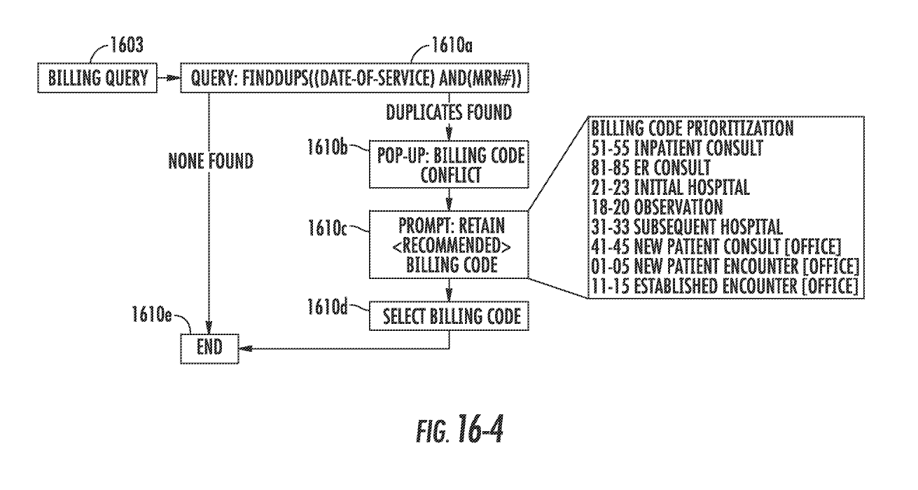

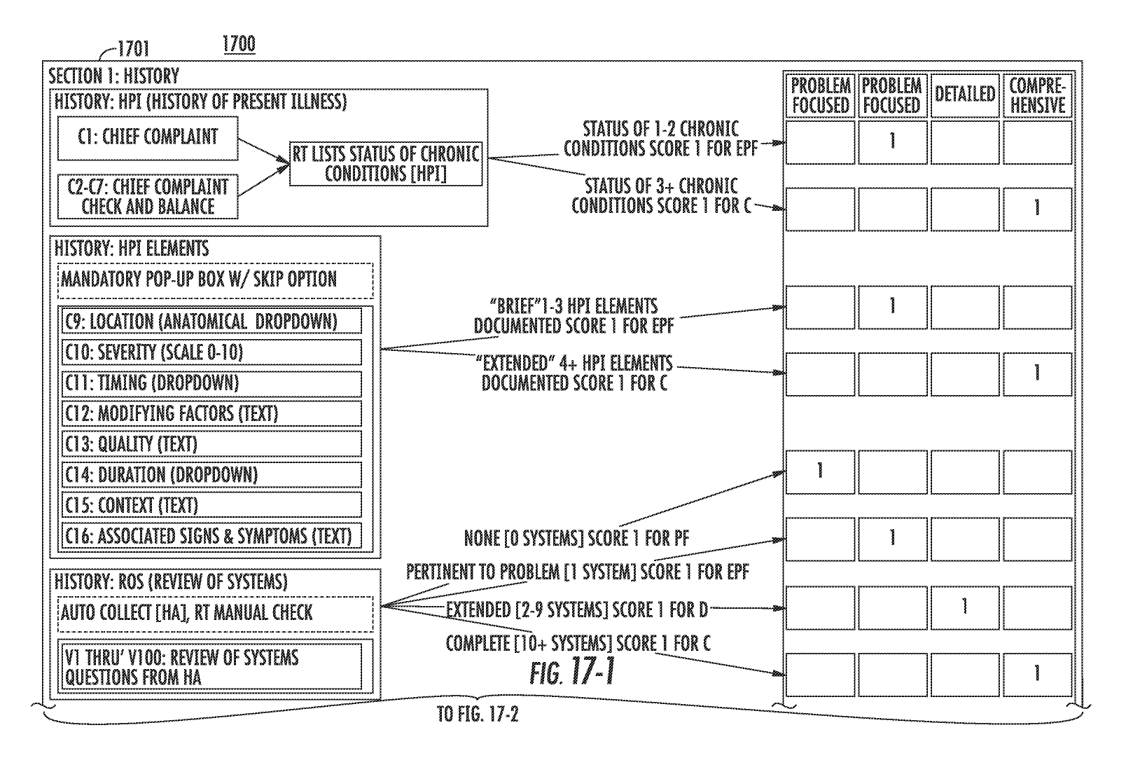

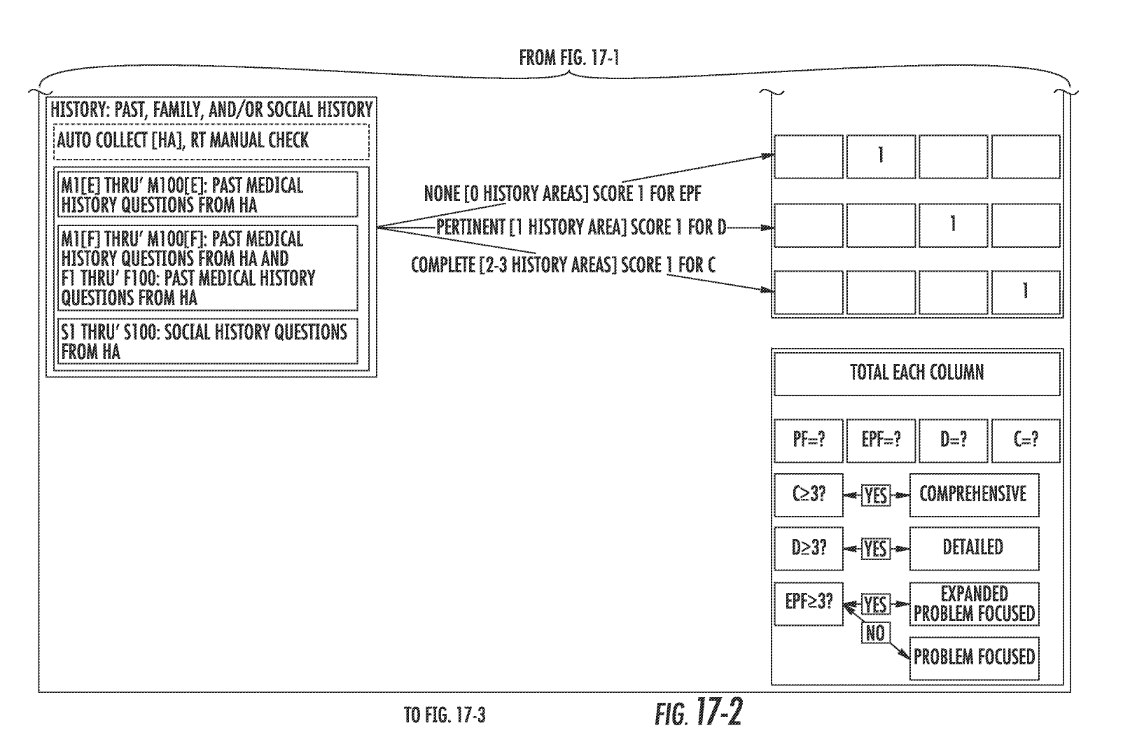

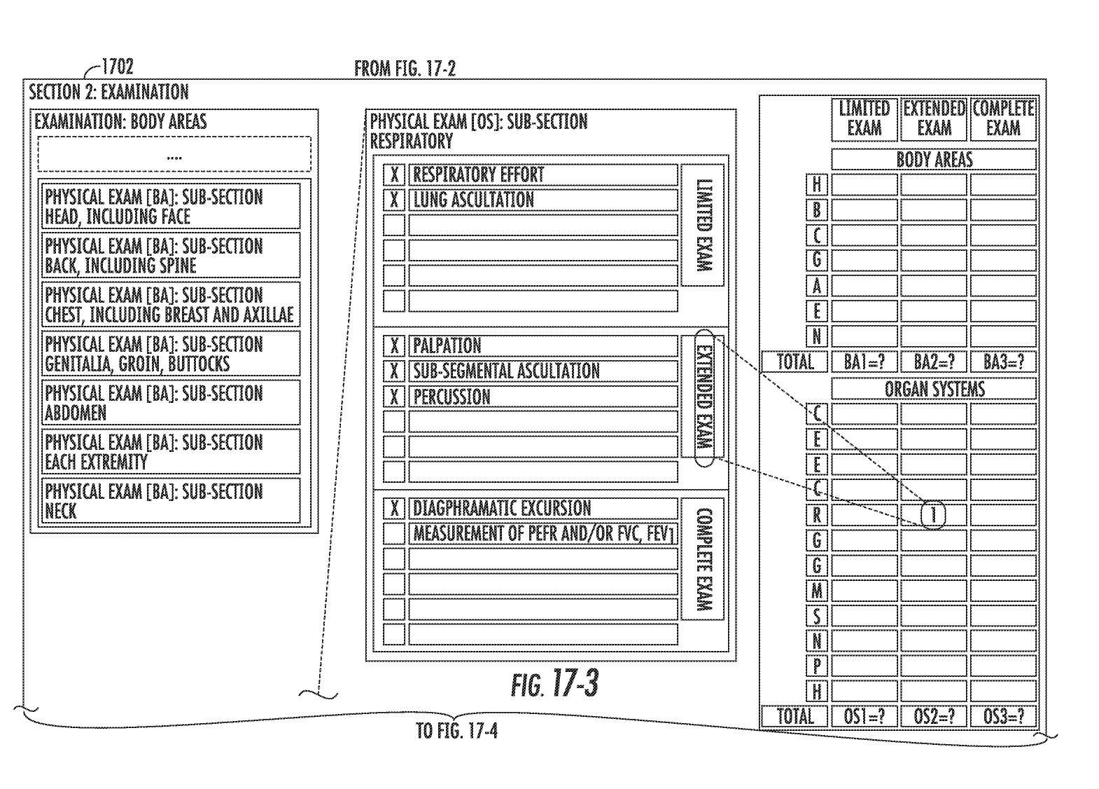

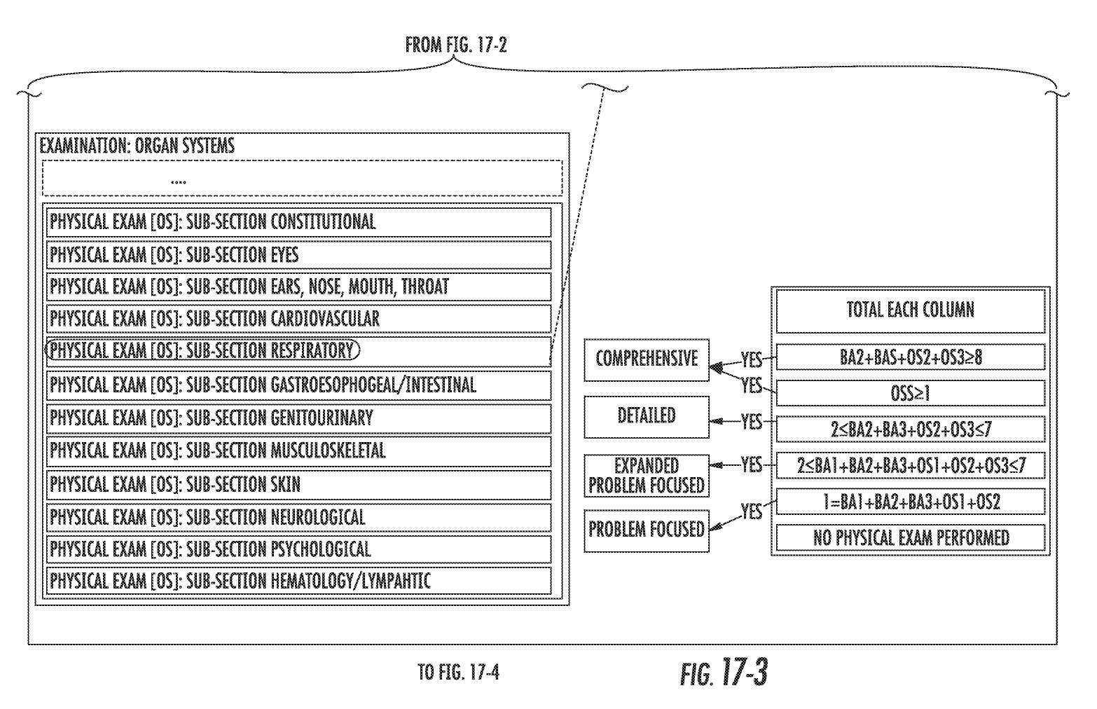

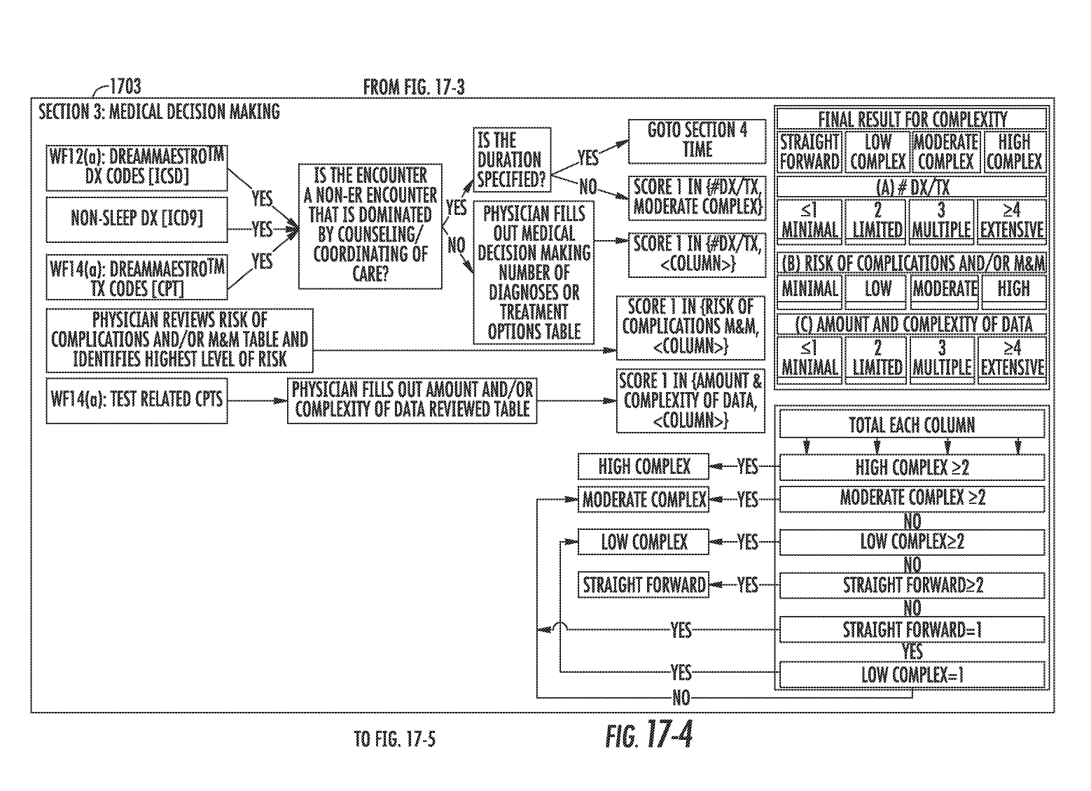

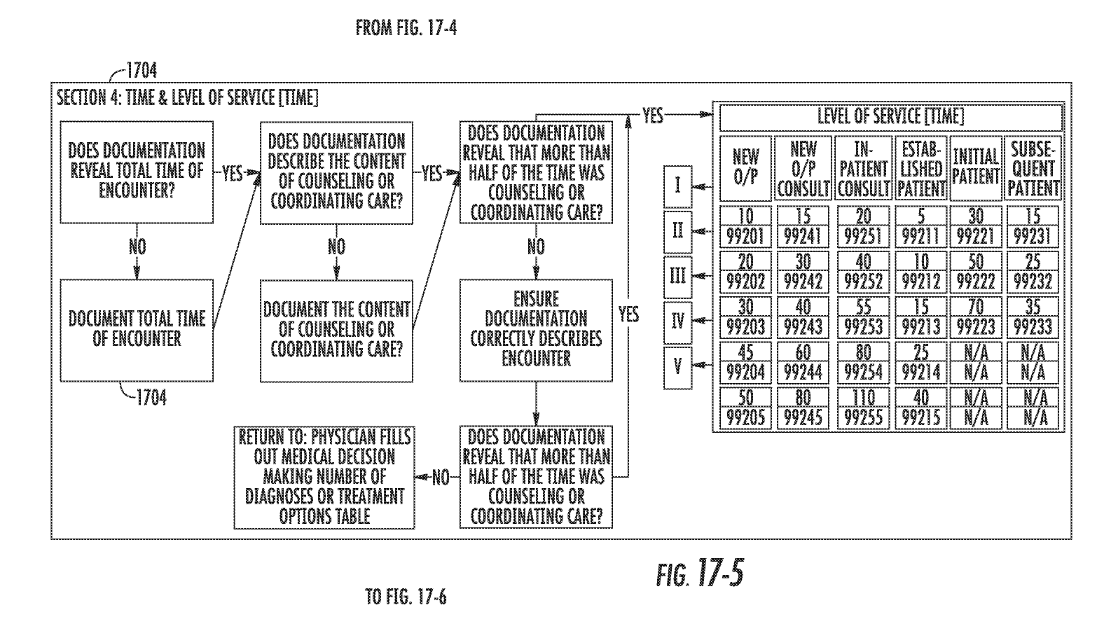

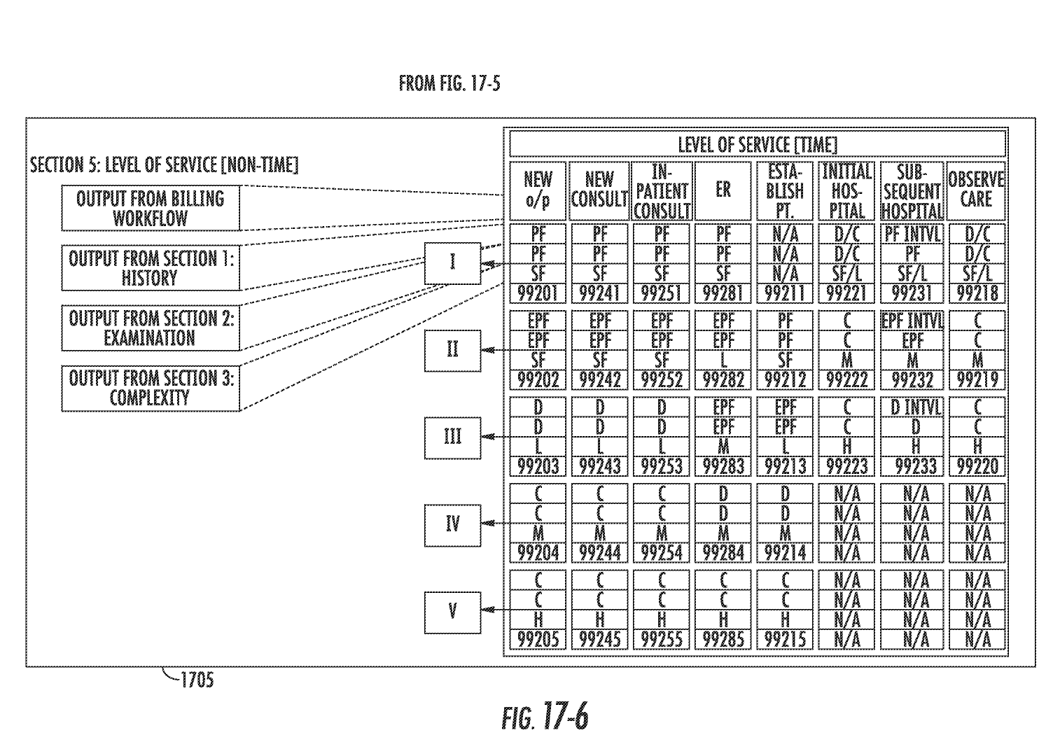

FIG. 17 illustrates a schematic diagram of emergency and medical (E&M) coding module 1700 from workflow 17 (WF17) which utilizes CMS standards to derive level-of-science of encounter from history, examination and medical decision making. E&M coding module 1700 suggests a CPT code for physician decision. CPT coding and ICD9 codes, derived from clinical evaluation pathways and diagnosis to treatment mapping are output to a billing system.

History module 1701 provides coding for history of the present illness (HPI), history of HPI elements, history review of HIP elements and past family and social history. Examination module 1702 provides coding for examination. Medical decision making module 1703 provides coding for medical decision making. Level-of-service-time module 1704 provides coding based on the service and time. Level-of-service-non-time module 1705 provides coding based on level of service and not time.

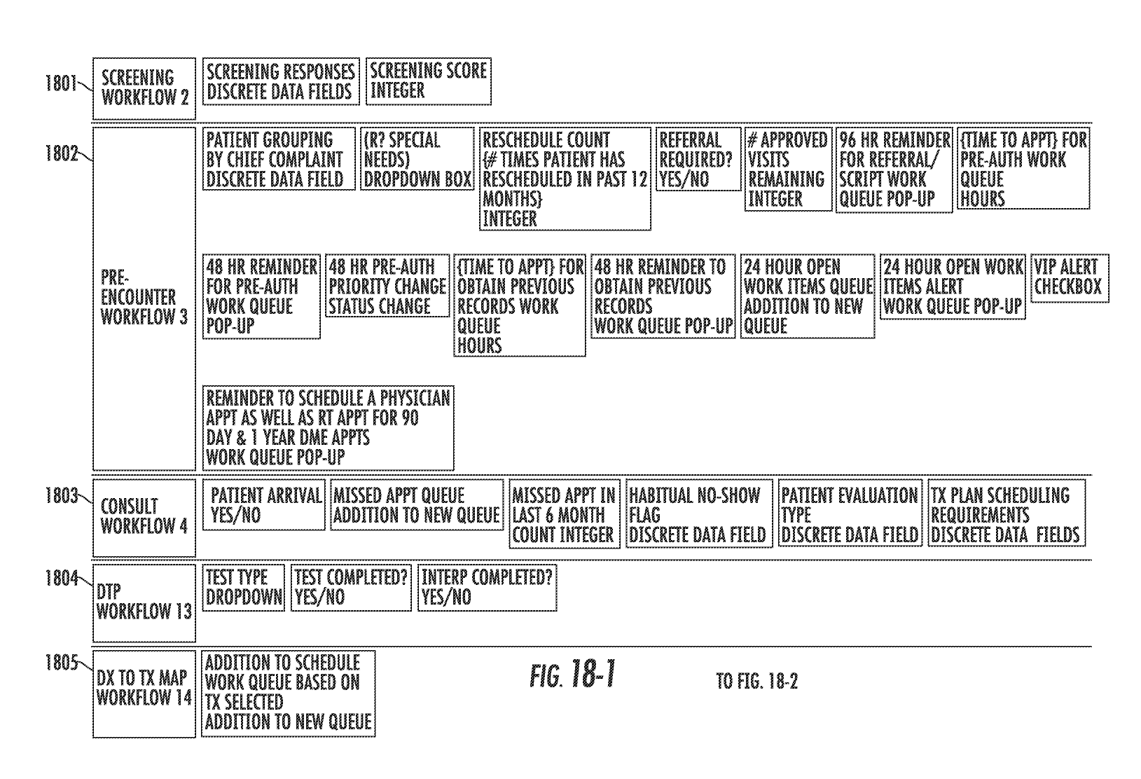

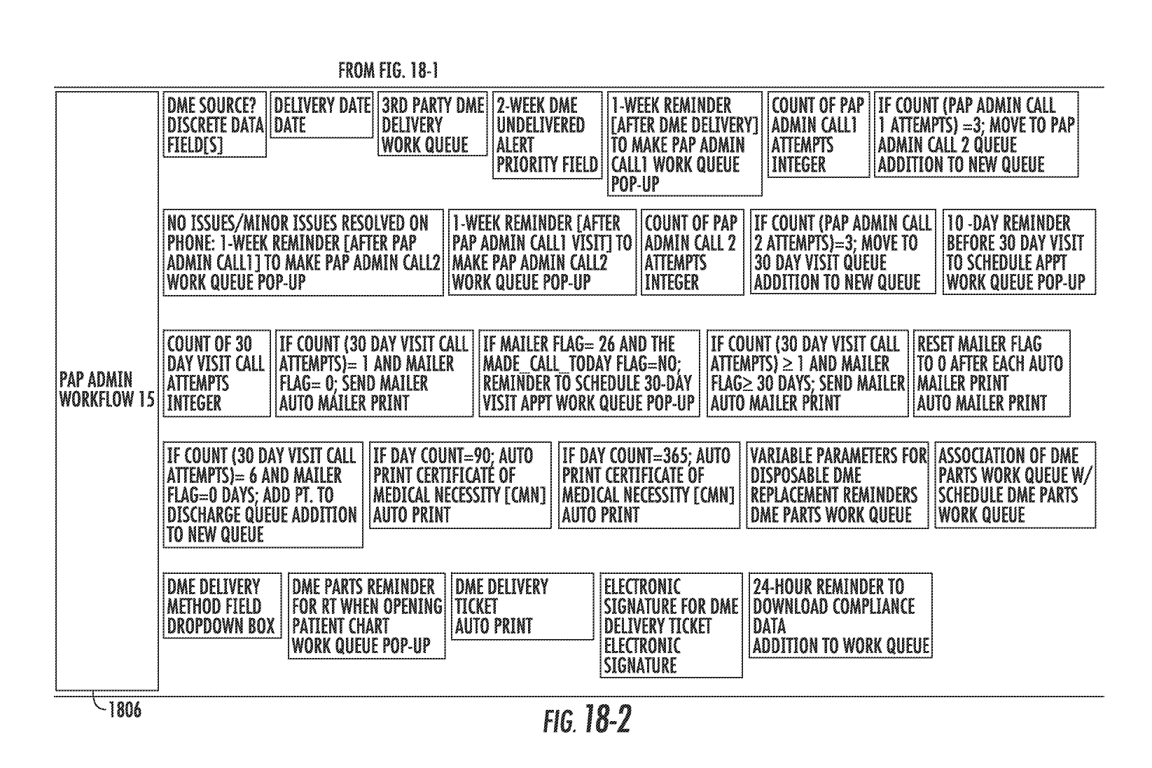

FIG. 18 is a schematic diagram of tracking module 1800 from workflow 18 (WF18) which provides tracking of a patient through workflows and populates electronic work queues from variable parameters. Block 1801 monitors screening module 200 from workflow 2. Block 1802 monitors pre-encounter module 300 from workflow 3. Block 1803 monitors encounter module 400 from workflow 4. Block 1804 monitors diagnostic testing pathway (DTP) module 1300 from workflow 13. Block 1805 monitors diagnosis-to-treatment map module 1400 from workflow 14. Block 1806 monitors positive airway pressure module 1500 from workflow 15.

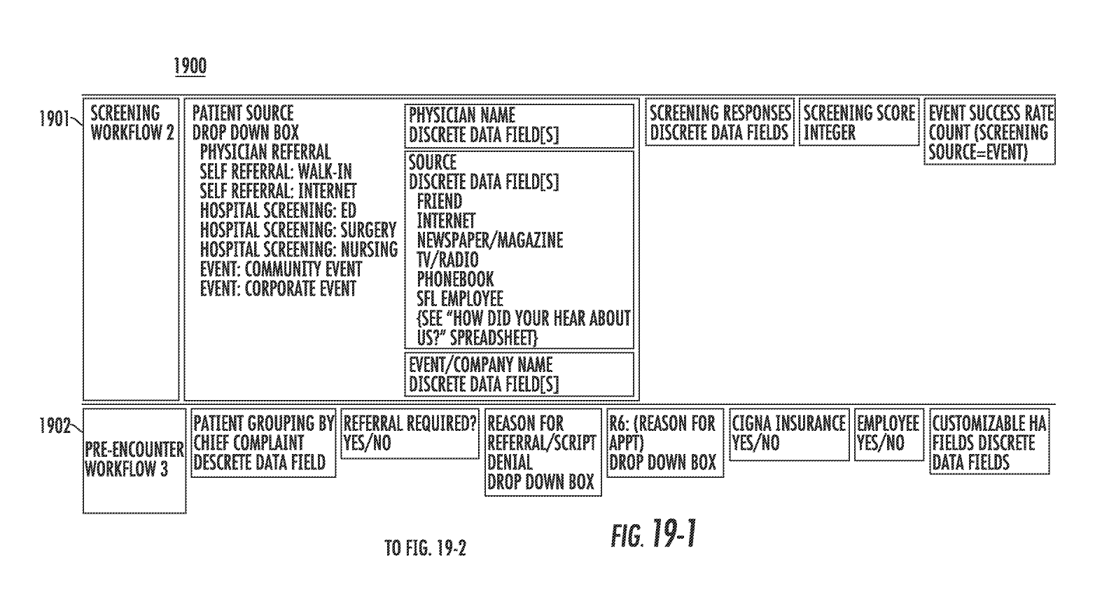

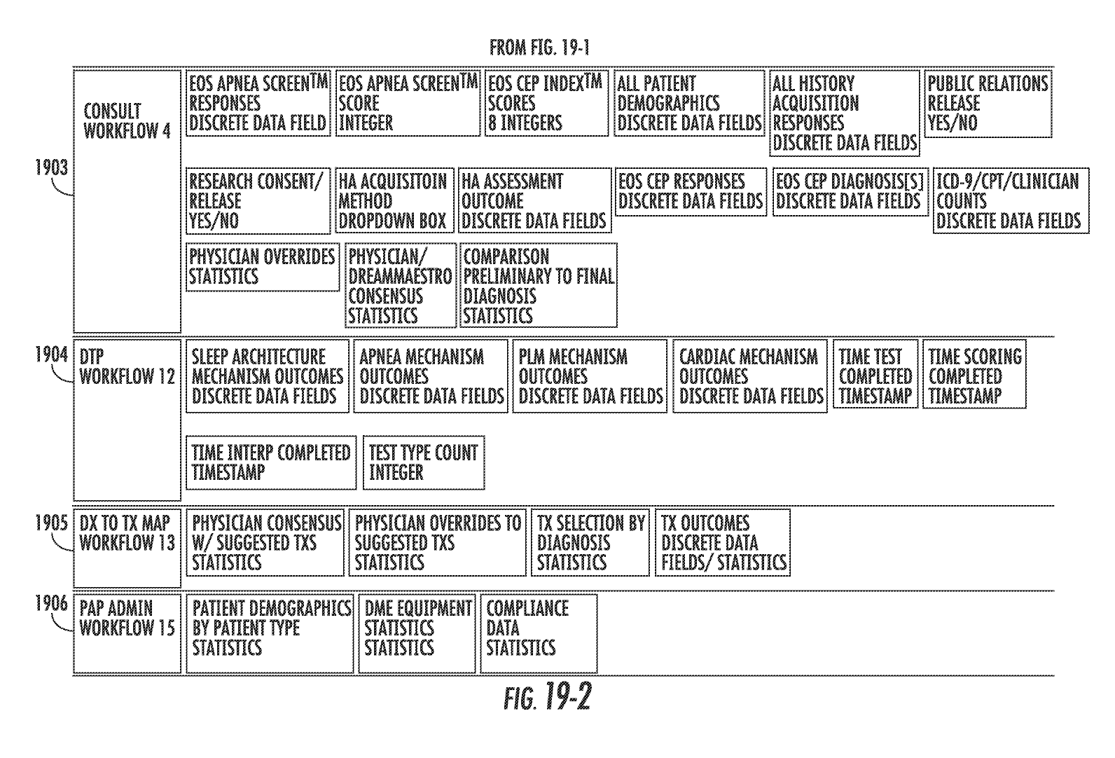

FIG. 19 is a schematic diagram of research module 1900 from workflow 19 (WF19) which provides a database query from evaluation tools, screening outcomes, patient demographics, disease process, treatment and patient outcomes.

Block 1901 provides a database query of screening module 200 from workflow 2. Block 1902 provides a database query of pre-encounter module 300 from workflow 3. Block 1903 provides a database query of encounter module 400 from workflow 4. Block 1904 provides a database query of diagnostic testing pathway (DTP) module 1300 from workflow 13. Block 1905 provides a database query of diagnosis-to-treatment map module 1400 from workflow 14. Block 1906 provides a database query of positive airway pressure module 1500 from workflow 15.

As discussed above, system 100 may be implemented over a variety of networks, communication links and protocols in order to achieve the dynamic input/output of data. System 100 can implement modules 200-1900. Accordingly, the present invention is further directed to a communication system for use in health information HIS 101. System 100 can include a central database, which includes multiple data fields populated with data. In particular, all or a portion of the various data points and above-described data fields could be added, modified and deleted in the database. In addition, a set of program instructions is configured to facilitate communication of data between one or more remote patient devices for entering data. In particular, communications device may be the Internet, a hardwired modem, a wireless modem or any other device that allows for the electronic communication of data from the remote patient device to and within the communication system. Because system 100 and the patient information interface are able to run on any existing browser, it may work on conventional browsers, such as Internet Explorer, Fire Fox, and the like. While any database or data structure is envisioned, one embodiment of system 100 will include the use of an SQL Server as the back-end database. In addition, system 100 will utilize the appropriate USB, PCMCIA and serial reader drivers.

### System Features

System features include;

i. Sleep disorder risk identification utilizing the sleep disorders screen 355.

ii. HIS component for patient eligibility criteria management. This includes apnea screen 360, patient history acquisition template (HA) 350, and HIS component 101 for patient registration and scheduling.

iii. Consultation management incorporating the HA, electronic patient consents, clinical encounter pathway, and diagnostic test pathway.

iv. DTP defines the clinical logic algorithm that standardizes the management and interpretation of diagnostic laboratory data and includes periodic limb movement index and sleep stage score.

v. Positive airway pressure program electronic management module.

vi. Electronic process for clinical evaluation and management billing which provides ICD9 coding and E/M coding.

vii. Electronic patient tracking through all program processes.

viii. Central repository for data mining patient electronic data for potential research initiatives and patient profiles.

It is to be understood that the above-described embodiments are illustrative of only a few of the many possible specific embodiments, which can represent applications of the principles of the invention. Numerous and varied other arrangements can be readily devised in accordance with these principles by those skilled in the art without departing from the spirit and scope of the invention.

What is claimed is:

1. A method for identification and management of a patient for one or more sleep disorders and determining one or more suggested diagnoses of sleep disorders comprising the steps of:
collecting sleep information of a patient directed to a sleep complaint;
scoring the sleep complaint based on evaluation of one or more clinical evaluation pathways;
acquiring history information and/or condition information from the patient as responses to diagnostic questions in a predetermined logic sequence;
merging the history information and/or condition information with the score of the sleep complaint into an electronic physical data examination form; and
correlating data from the physical data examination form and the responses to diagnostic questions to provide a suggested diagnosis of one or more sleep disorder conditions.

2. The method of claim 1 wherein the clinical evaluation pathway is a movement clinical encounter pathway.

3. The method of claim 1 wherein the clinical evaluation pathway is an insomnia clinical encounter pathway.

4. The method of claim 1 wherein the clinical evaluation pathway is a breathing clinical encounter pathway.

5. The method of claim 1 wherein the clinical evaluation pathway is a hypersomnia clinical encounter pathway.

6. The method of claim 1 wherein the clinical evaluation pathway is a parasomnia clinical encounter pathway.

7. The method of claim 1 wherein the clinical evaluation pathway is a circadian clinical encounter pathway.

8. The method of claim 1 wherein the clinical evaluation pathway is a variant clinical encounter pathway.

9. The method of claim 1 wherein the clinical evaluation pathway is a sleep disorder related to other conditions clinical encounter pathway.

10. The method of claim 1 further comprising the step of:
determining a sleepiness score for daytime sleepiness and merging the sleepiness score into the physical data examination form.

11. The method of claim 1 further comprising the step of:
registering the patient before said step of collecting sleep information.

12. The method of claim 1 further comprising the step of:
scheduling the patient for a sleep disorder appointment.

13. The method of claim 1 further comprising the step of:
pre-certifying the patient.

14. The method of claim 1 wherein the history information is acquired by completing an electronic patient history acquisition template.

15. The method of claim 14 wherein the electronic patient history acquisition template includes a sleep disorder screen.

16. The method of claim 14 wherein the electronic patient history acquisition template includes an apnea screen template.

17. The method of claim 14 wherein the electronic patient history acquisition template includes one or more clinical encounter pathway scores directed to the one or more clinical evaluation pathways.

18. The method of claim 1 further comprising the step of:
prompting a respiratory therapist to review the physical data examination form, history information and/or condition information and provide a differential diagnosis and a suggested treatment plan.

19. The method of claim 1 further comprising:
providing patient education materials based on the suggested diagnosis.

20. The method of claim 1 wherein said condition information is obtained from one or more polysysmograph data, positive airway pressure titration, periodic limb movement, cardiac and sleep architecture.

21. The method of claim 1 wherein said condition information is determined from multiple sleep latency testing and/or wakefulness testing.

22. The method of claim 1 wherein the condition information comprises sleep efficiency information in one or more sleep stages.

23. The method of claim 1 further comprising the step of:
evaluating a patient for positive airway pressure equipment based on said suggested diagnosis;
delivering said equipment; and
tracking electronically of said equipment during use by the patient.

24. The method of claim 1 further comprising emergency and medical coding of said history information and the suggested diagnosis of one or more sleep disorder conditions.

25. The method of claim 1 wherein said history information is acquired through a graphical user interface.

26. The method of claim 1 further comprising the step of:
providing a suggested treatment plan based on the suggested diagnosis of one or more sleep disorder conditions.

27. A system for identification and management of a patient for one or more sleep disorders and determining one or more suggested diagnoses of sleep disorders comprising:
means for collecting sleep information of a patient directed to a sleep complaint;
means for scoring the sleep complaint based on evaluation of one or more clinical evaluation pathways;
means for acquiring history information and/or condition information from the patient as responses to diagnostic questions in a predetermined logic sequence;
means for merging the history information and/or condition information with the score of the sleep complaint into an electronic physical data examination form; and
means for correlating data from the physical data examination form and the responses to diagnostic questions to provide a suggested diagnosis of one or more sleep disorder conditions.

28. The system of claim 27 further comprising:
means for determining a sleepiness score for daytime sleepiness and merging the sleepiness score into the physical data examination form.
